SEQUENCE LISTING (301 sequences)

SEQ ID NO: 1 - DNA, 1122 bp, synthetic construct
  source 1..1122: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggctaa

SEQ ID NO: 2 - protein, 373 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGG

SEQ ID NO: 3 - DNA, 1152 bp, synthetic construct
  source 1..1152: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcacgggggttaa

SEQ ID NO: 4 - protein, 383 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHGG

SEQ ID NO: 5 - DNA, 1152 bp, synthetic construct; identical to SEQ ID NO: 3
  source 1..1152: mol_type = other DNA

SEQ ID NO: 6 - protein, 383 aa, synthetic construct; identical to SEQ ID NO: 4

SEQ ID NO: 7 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 8 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 9 - DNA, 1122 bp, synthetic construct; identical to SEQ ID NO: 1
  source 1..1122: mol_type = other DNA

SEQ ID NO: 10 - protein, 373 aa, synthetic construct; identical to SEQ ID NO: 2

SEQ ID NO: 11 - DNA, 1146 bp, synthetic construct
  source 1..1146: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcacgggggttaa

SEQ ID NO: 12 - protein, 381 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHGG

SEQ ID NO: 13 - DNA, 1161 bp, synthetic construct
  source 1..1161: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttggagaacctgtacttccaatccggcggtcatcatcaccaccatcacggcggccaccaccaccaccaccactga

SEQ ID NO: 14 - protein, 386 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLENLYFQSGGHHHHHHGGHHHHHH

SEQ ID NO: 15 - DNA, 1179 bp, synthetic construct
  source 1..1179: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttggagaacctgtacttccaatccggcggtcatcatcaccaccatcacggcggccatcaccatcaccatcaccatcaccatcacgggggttaa

SEQ ID NO: 16 - protein, 392 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLENLYFQSGGHHHHHHGGHHHHHHHHHHGG

SEQ ID NO: 17 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 18 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 19 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagttcgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 20 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKVRSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 21 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagttcgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 22 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKVRSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 23 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagttcgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 24 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKVRSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 25 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagttcgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 26 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKVRSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 27 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttctcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 28 - protein, 363 aa, synthetic construct
MAQTPAFDKPKVELHVHLDGSIKPETILYYGRRRGIALPANTAEGLLNVIGMDKPLTLPDFLAKFDYYMPAIAGSREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 29 - DNA, 1122 bp, synthetic construct
  source 1..1122: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttctcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggctaa

SEQ ID NO: 30 - protein, 373 aa, synthetic construct
MAQTPAFDKPKVELHVHLDGSIKPETILYYGRRRGIALPANTAEGLLNVIGMDKPLTLPDFLAKFDYYMPAIAGSREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGG

SEQ ID NO: 31 - DNA, 1092 bp, synthetic construct
  source 1..1092: mol_type = other DNA
atggctcaaactccggccttcaataagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 32 - protein, 363 aa, synthetic construct [sequence omitted]

SEQ ID NO: 33 - DNA, 1158 bp, synthetic construct
  source 1..1158: mol_type = other DNA
atggctcaaactccggccttcaataagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 34 - protein, 385 aa, synthetic construct
MAQTPAFNKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 35 - DNA, 1092 bp, synthetic construct
  source 1..1092: mol_type = other DNA
atggctcaaactccggccttcaataagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 36 - protein, 363 aa, synthetic construct
MAQTPAFNKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 37 - DNA, 1158 bp, synthetic construct
  source 1..1158: mol_type = other DNA
atggctcaaactccggccttcaataagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 38 - protein, 385 aa, synthetic construct
MAQTPAFNKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 39 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggcgtttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 40 - protein, 363 aa, synthetic construct
MAQTPAFDKPKVELHVHLDGSIKPETILYYGRRRGIALPANTAEGLLNVIGMDKPLTLPAFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 41 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggcgtttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 42 - protein, 385 aa, synthetic construct
MAQTPAFDKPKVELHVHLDGSIKPETILYYGRRRGIALPANTAEGLLNVIGMDKPLTLPAFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 43 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgctgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 44 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGAETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 45 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgctgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 46 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGAETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 47 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtaaagtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 48 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGKVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 49 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtaaagtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 50 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGKVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 51 - DNA, 1161 bp, synthetic construct
  source 1..1161: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtaaagtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttggagaacctgtacttccaatccggcggtcatcatcaccaccatcacggcggccaccaccaccaccaccactga

SEQ ID NO: 52 - protein, 386 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGKVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLENLYFQSGGHHHHHHGGHHHHHH

SEQ ID NO: 53 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaagaactgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 54 - protein, 363 aa, synthetic construct
MAQTPAFDKPKVELHVHLDGSIKPETILYYGRRRGIALPANTAEELLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 55 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaagaactgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 56 - protein, 385 aa, synthetic construct
MAQTPAFDKPKVELHVHLDGSIKPETILYYGRRRGIALPANTAEELLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 57 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaagaactgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 58 - protein, 363 aa, synthetic construct
MAQTPAFDKPKVELHVHLDGSIKPETILYYGRRRGIALPANTAEELLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 59 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaagaactgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 60 - protein, 385 aa, synthetic construct
MAQTPAFDKPKVELHVHLDGSIKPETILYYGRRRGIALPANTAEELLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 61 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgaatcagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 62 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVNQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 63 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgaatcagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 64 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVNQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 65 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgaatcagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 66 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVNQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 67 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgaatcagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 68 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVNQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 69 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaagtgtgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 70 - protein, 363 aa, synthetic construct [sequence omitted]

SEQ ID NO: 71 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaagtgtgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 72 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEVCPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 73 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtggtccgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 74 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVVRLKNDQANYSLNTDDPLIFKSTLDTDYQMTKRDMGFTEEEFKRLNINAAKSSFLPEDEKRELLDLLYKAYGMPPSASAGQNL

SEQ ID NO: 75 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtggtccgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 76 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVVRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 77 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 78 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 79 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 80 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 81 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaagtgtgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 82 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEVCPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 83 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaagtgtgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 84 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEVCPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 85 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtggtccgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 86 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVVRLKNDQANYSLNTDDPLIFKSTLDTDYQMTKRDMGFTEEEFKRLNINAAKSSFLPEDEKRELLDLLYKAYGMPPSASAGQNL

SEQ ID NO: 87 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtggtccgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 88 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVVRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 89 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 90 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 91 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 92 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 93 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtggaagcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 94 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVEAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 95 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtggaagcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 96 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVEAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 97 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgaaagcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 98 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVKAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 99 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgaaagcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 100 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVKAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 101 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgcaggcctatgcggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 102 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVQAYAEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 103 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgcaggcctatgcggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 104 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVQAYAEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 105 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgcaggcctatgaagaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 106 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVQAYEEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 107 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttatttccaggacacgtgcaggcctatgaagaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 108 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLFPGHVQAYEEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 109 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgttttaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 110 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRFKNDQANYSLNTDDPLIFKSTLDTDYQMTKRDMGFTEEEFKRLNINAAKSSFLPEDEKRELLDLLYKAYGMPPSASAGQNL

SEQ ID NO: 111 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgttttaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 112 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRFKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 113 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattgaagggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 114 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIEGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 115 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattgaagggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 116 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIEGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 117 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtacaatcagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 118 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYNQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 119 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtacaatcagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 120 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYNQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 121 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccgtcagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 122 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYRQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 123 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccgtcagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 124 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYRQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 125 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaagagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 126 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQEQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 127 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaagagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 128 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQEQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 129 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagaaagaacttttagatctgctgtataaggcgtacggtatgccgccttcggcaagcgcgggccagaacttgtaa

SEQ ID NO: 130 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKKELLDLLYKAYGMPPSASAGQNL

SEQ ID NO: 131 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagaaagaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 132 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKKELLDLLYKAYGMPPSASAGQNLGGHHHHHHGGHHHHHHHHHHGG

SEQ ID NO: 133 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 134 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 135 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 136 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 137 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgttttaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 138 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRFKNDQANYSLNTDDPLIFKSTLDTDYQMTKRDMGFTEEEFKRLNINAAKSSFLPEDEKRELLDLLYKAYGMPPSASAGQNL

SEQ ID NO: 139 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgttttaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 140 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRFKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 141 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccgataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 142 - protein, 363 aa, synthetic construct
MAQTPAFDKPKVELHVHLDGSIKPETILYYGRRRGIALPADTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 143 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccgataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 144 - protein, 385 aa, synthetic construct
MAQTPAFDKPKVELHVHLDGSIKPETILYYGRRRGIALPADTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 145 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccgataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 146 - protein, 363 aa, synthetic construct
MAQTPAFDKPKVELHVHLDGSIKPETILYYGRRRGIALPADTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 147 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccgataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 148 - protein, 385 aa, synthetic construct
MAQTPAFDKPKVELHVHLDGSIKPETILYYGRRRGIALPADTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 149 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccgattggagcccggaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 150 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPDWSPEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 151 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccgattggagcccggaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 152 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPDWSPEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 153 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 154 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 155 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 156 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 157 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 158 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 159 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 160 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 161 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccggaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 162 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 163 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccggaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 164 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 165 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccggaagtagtagagttgtgcaaaaagtaccaacagcaaactgtcgttgcaatcgatctggcgggtgacgaaacgattgaagggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 166 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPEVVELCKKYQQQTVVAIDLAGDETIEGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 167 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagccctcatggagcccggaagtagtagagttgtgcaaaaagtaccaacagcaaactgtcgttgcaatcgatctggcgggtgacgaaacgattgaagggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 168 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPSWSPEVVELCKKYQQQTVVAIDLAGDETIEGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 169 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagccttgaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 170 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSLEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 171 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagccttgaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 172 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSLEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 173 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcagcgaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 174 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSSEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 175 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcagcgaagtagtagagttgtgcaaaaagtaccaacagcaaactgtcgttgcaatcgatctggcgggtgacgaaacgattccggggagttctttatttccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 176 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSSEVVELCKKYQQQTVVAIDLAGDETIPGSSLFPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 177 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattgaagggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 178 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIEGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 179 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattgaagggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 180 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIEGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 181 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgcaagcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 182 - protein, 363 aa, synthetic construct
MAQTPAFDKPKVELHVHLDGSIKPETILYYGRKRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 183 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgcaagcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 184 - protein, 385 aa, synthetic construct
MAQTPAFDKPKVELHVHLDGSIKPETILYYGRKRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 185 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgcaagcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 186 - protein, 363 aa, synthetic construct
MAQTPAFDKPKVELHVHLDGSIKPETILYYGRKRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 187 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgcaagcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 188 - protein, 385 aa, synthetic construct
MAQTPAFDKPKVELHVHLDGSIKPETILYYGRKRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 189 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacaggacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtga

SEQ ID NO: 190 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGEQDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 191 - DNA, 1161 bp, synthetic construct
  source 1..1161: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacaggacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttggagaacctgtacttccaatccggcggtcatcatcaccaccatcacggcggccaccaccaccaccaccactga

SEQ ID NO: 192 - protein, 386 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGEQDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLENLYFQSGGHHHHHHGGHHHHHH

SEQ ID NO: 193 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagaaagaacttttagatctgctgtataaggcgtacggtatgccgccttcggcaagcgcgggccagaacttgtaa

SEQ ID NO: 194 - protein, 363 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKKELLDLLYKAYGMPPSASAGQNL

SEQ ID NO: 195 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagaaagaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 196 - protein, 385 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKKELLDLLYKAYGMPPSASAGQNLGGHHHHHHGGHHHHHHHHHHGG

SEQ ID NO: 197 - DNA, 1092 bp, synthetic construct
  source 1..1092: mol_type = other DNA
atggctcaaactccggccttcgacaagccgaaagtcgaactccatgtacatcttgatggagccataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 198 - protein, 363 aa, synthetic construct
MAQTPAFDKPKVELHVHLDGAIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 199 - DNA, 1158 bp, synthetic construct
  source 1..1158: mol_type = other DNA
atggctcaaactccggccttcgacaagccgaaagtcgaactccatgtacatcttgatggagccataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 200 - protein, 385 aa, synthetic construct
MAQTPAFDKPKVELHVHLDGAIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 201 - DNA, 1092 bp, synthetic construct
  source 1..1092: mol_type = other DNA
atggctcaaactccggccttcgacaagccgaaagtcgaactccatgtacatcttgatggagccataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 202 - protein, 363 aa, synthetic construct
MAQTPAFDKPKVELHVHLDGAIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 203 - DNA, 1158 bp, synthetic construct
  source 1..1158: mol_type = other DNA
atggctcaaactccggccttcgacaagccgaaagtcgaactccatgtacatcttgatggagccataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 204 - protein, 385 aa, synthetic construct
MAQTPAFDKPKVELHVHLDGAIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 205 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctctcccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 206 - protein, 363 aa, synthetic construct
MAQTPAFDKPKVELHVHLDGSIKPETILYYGRRRGIALPANTAEGLLNVIGMDKPLSLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 207 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctctcccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 208 - protein, 385 aa, synthetic construct
MAQTPAFDKPKVELHVHLDGSIKPETILYYGRRRGIALPANTAEGLLNVIGMDKPLSLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 209 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctctcccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 210 - protein, 363 aa, synthetic construct
MAQTPAFDKPKVELHVHLDGSIKPETILYYGRRRGIALPANTAEGLLNVIGMDKPLSLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 211 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctctcccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 212 - protein, 385 aa, synthetic construct
MAQTPAFDKPKVELHVHLDGSIKPETILYYGRRRGIALPANTAEGLLNVIGMDKPLSLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 213 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacatcatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 214 - protein, 363 aa, synthetic construct
MAQTPAFDKPKVELHVHLDGSIKPETILYYGRRRGIALPANTAEGLLNIIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 215 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacatcatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 216 - protein, 385 aa, synthetic construct
MAQTPAFDKPKVELHVHLDGSIKPETILYYGRRRGIALPANTAEGLLNIIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 217 - DNA, 1092 bp, synthetic construct
  source 1..1092: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacatcatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 218 - protein, 363 aa, synthetic construct
MAQTPAFDKPKVELHVHLDGSIKPETILYYGRRRGIALPANTAEGLLNIIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 219 - DNA, 1158 bp, synthetic construct
  source 1..1158: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacatcatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccggaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggccatcaccatcaccatcaccatcaccatcacgggggttaa

SEQ ID NO: 220 - protein, 385 aa, synthetic construct
MAQTPAFDKPKVELHVHLDGSIKPETILYYGRRRGIALPANTAEGLLNIIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPEVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGGHHHHHHHHHHGG

SEQ ID NO: 221 - DNA, 1062 bp, synthetic construct
  source 1..1062: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgtaa

SEQ ID NO: 222 - protein, 353 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGM

SEQ ID NO: 223 - DNA, 1128 bp, synthetic construct
  source 1..1128: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgggcggtcatcatcaccaccatcacggcggccatcaccatcaccatcaccatcaccatcacgggggttaa

SEQ ID NO: 224 - protein, 375 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMGGHHHHHHGGHHHHHHHHHHGG

SEQ ID NO: 225 - DNA, 1131 bp, synthetic construct
  source 1..1131: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatggagaacctgtacttccaatccggcggtcatcatcaccaccatcacggcggccaccaccaccaccaccactga

SEQ ID NO: 226 - protein, 376 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMENLYFQSGGHHHHHHGGHHHHHH

SEQ ID NO: 227 - DNA, 1149 bp, synthetic construct
  source 1..1149: mol_type = other DNA
atggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatggagaacctgtacttccaatccggcggtcatcatcaccaccatcacggcggccatcaccatcaccatcaccatcaccatcacgggggttaa

SEQ ID NO: 228 - protein, 382 aa, synthetic construct
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMENLYFQSGGHHHHHHGGHHHHHHHHHHGG

SEQ ID NO: 229 - DNA, 2367 bp, synthetic construct
  source 1..2367: mol_type = other DNA
atgggcagcagccatcatcatcatcatcacagcagcggcctggtgccgcgcggcagccatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcccaccatcaccatcaccacgcgaattcggtaccgctggttccgcgtggatctgagaacctgtacttccaatccatggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggctaa

SEQ ID NO: 230 - protein, 788 aa, synthetic construct
MGSSHHHHHHSSGLVPRGSHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSHHHHHHANSVPLVPRGSENLYFQSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGG

SEQ ID NO: 231 - DNA, 1122 bp, synthetic construct
  source 1..1122: mol_type = other DNA
atgggcggtcatcatcaccaccatcacggcggcgctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttgtcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgtaa

SEQ ID NO: 232 - protein, 373 aa, synthetic construct
MGGHHHHHHGGAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 233 - DNA, 1122 bp, synthetic construct
  source 1..1122: mol_type = other DNA
atgggcggtcatcatcaccaccatcacggcggcgctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttctcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgtaa

SEQ ID NO: 234 - protein, 373 aa, synthetic construct
MGGHHHHHHGGAQTPAFNKPKVELHVHLDGAIKPETILYYGRKRGIALPADTPEELQNIIGMDKPLSLPEFLAKFDYYMPAIAGSREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 235 - DNA, 1122 bp, synthetic construct
  source 1..1122: mol_type = other DNA
atgggcggtcatcatcaccaccatcacggcggcgctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 236 - protein, 373 aa, synthetic construct
MGGHHHHHHGGAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 237 - DNA, 1122 bp, synthetic construct
  source 1..1122: mol_type = other DNA
atgggcggtcatcatcaccaccatcacggcggcgctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttctcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgtaa

SEQ ID NO: 238 - protein, 373 aa, synthetic construct
MGGHHHHHHGGAQTPAFDKPKVELHVHLDGSIKPETILYYGRRRGIALPANTAEGLLNVIGMDKPLTLPDFLAKFDYYMPAIAGSREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 239 - DNA, 1089 bp, synthetic construct
  source 1..1089: mol_type = other DNA
atgggcggtcatcatcaccaccatcacggcggcgcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttgtcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaataa

SEQ ID NO: 240 - protein, 362 aa, synthetic construct
MGGHHHHHHGGAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 241 - DNA, 1089 bp, synthetic construct
  source 1..1089: mol_type = other DNA
atgggcggtcatcatcaccaccatcacggcggcgcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttctcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaataa

SEQ ID NO: 242 - protein, 362 aa, synthetic construct
MGGHHHHHHGGAQTPAFNKPKVELHVHLDGAIKPETILYFGKKRGIALPADTVEELRNIIGMDKPLSLPGFLAKFDYYMPVIAGSREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 243 - DNA, 1092 bp, synthetic construct
  source 1..1092: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttgtcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgtaa

SEQ ID NO: 244 - protein, 363 aa, synthetic construct
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 245 - DNA, 1092 bp, synthetic construct
  source 1..1092: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttctcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgtaa

SEQ ID NO: 246 - protein, 363 aa, synthetic construct
MAQTPAFNKPKVELHVHLDGAIKPETILYYGRKRGIALPADTPEELQNIIGMDKPLSLPEFLAKFDYYMPAIAGSREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 247 - DNA, 1122 bp, synthetic construct
  source 1..1122: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttctcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgggcggtcatcatcaccaccatcacggcggctaa

SEQ ID NO: 248 - protein, 373 aa, synthetic construct
MAQTPAFNKPKVELHVHLDGAIKPETILYYGRKRGIALPADTPEELQNIIGMDKPLSLPEFLAKFDYYMPAIAGSREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCLGGHHHHHHGG

SEQ ID NO: 249 - DNA, 1122 bp, synthetic construct
  source 1..1122: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttgtcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgggcggtcatcatcaccaccatcacggcggctaa

SEQ ID NO: 250 - protein, 373 aa, synthetic construct
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCLGGHHHHHHGG

SEQ ID NO: 251 - DNA, 1146 bp, synthetic construct
  source 1..1146: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttgtcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgggcggtcatcatcaccaccatcacggcggccatcaccatcaccatcacgggggttaa

SEQ ID NO: 252 - protein, 381 aa, synthetic construct
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCLGGHHHHHHGGHHHHHHGG

SEQ ID NO: 253 - DNA, 1158 bp, synthetic construct
  source 1..1158: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttgtcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttgggcggtcatcatcaccaccatcacggcggccatcaccatcaccatcaccatcaccatcacgggggttaa

SEQ ID NO: 254 - protein, 385 aa, synthetic construct
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCLGGHHHHHHGGHHHHHHHHHHGG

SEQ ID NO: 255 - DNA, 1179 bp, synthetic construct
  source 1..1179: mol_type = other DNA
atggctcaaactcccgccttcaataagcctaaagtcgagctccacgttcatctggacggggcgataaaaccagaaacaatcctgtactacggtcgcaaacgtgggattgccctgccggcagatacgccggaagagctgcagaacatcattggcatggataagccgctgagtcttcctgaatttttggctaaattcgactactatatgcccgccatcgcaggttgtcgcgaagcggtgaaacgtattgcgtacgagtttgtagaaatgaaagctaaggatggcgtggtctatgttgaagtgcgctactcaccacacctcttagcaaatagcaaagtagagccgatcccgtggaaccaggccgaaggcgacctgaccccggatgaagtcgtgtccctggttaatcaaggtctgcaggagggcgaacgtgatttcggcgtgaaagtacgcagtatattatgctgtatgcgtcatcagccttcgtggagctccgaagtcgttgagctttgcaaaaagtatcgcgaacaaaccgttgtggcgatcgacctcgcaggtgatgaaactattgagggcagttctttgtttccaggccacgtcaaagcttacgcggaagcggttaaatcaggagtgcatcgtacagtgcacgccggtgaagtgggctcggcaaacgtcgtaaaagaggcggttgatacgttaaagaccgaacgcctgggacatggttatcacaccctggaagacgcaacgctgtacaatcgtctgcgccaggagaacatgcatttcgaagtgtgtccctggagctcgtatctcactggagcctggaaaccggatacagaacatccggtggttcgttttaaaaatgaccaggtaaactattctcttaatacggatgatccgttgattttcaaatcaaccttagacaccgattatcaaatgacgaagaacgagatggggtttaccgaagaagagtttaaacgcctgaatattaacgcggcgaaaagcagctttctgcctgaagatgaaaaaaaggaattactggatctgctttataaagcgtatggtatgccatctccggcctccgctgaacagtgcttggagaacctgtacttccaatccggcggtcatcatcaccaccatcacggcggccatcaccatcaccatcaccatcaccatcacgggggttaa

SEQ ID NO: 256 - protein, 392 aa, synthetic construct
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCLENLYFQSGGHHHHHHGGHHHHHHHHHHGG

SEQ ID NO: 257 - DNA, 1803 bp, synthetic construct
  source 1..1803: mol_type = other DNA
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccaatggctcaaactccggccttcgacaagccgaaagtcgaactccatgtacatcttgatggaagtataaaaccggagacgatcctgtattatgggcgccgtcgcggtattgctttacctgccaataccgcagaaggtctgctgaacgttatcggcatggacaaacccctcacccttccggattttctggctaagttcgattattacatgccggcgattgctggttgtcgtgaagccatcaaacgcattgcttatgagtttgttgaaatgaaagcgaaagaaggcgtggtctacgttgaagtacgttattctcctcacctgttagctaattccaaggtggagccaatcccgtggaaccaagccgaaggagatctgacaccagacgaagttgtcgcactggtgggccagggtctccaggagggggaacgcgacttcggcgtgaaagcccgttcgattctttgctgtatgcgccatcagcccaattggagcccgaaagtagtagagttgtgcaaaaagtaccaacagcaaactgtcgttgcaatcgatctggcgggtgacgaaacgattccggggagttctttactgccaggacacgtgcaggcctatcaggaagcggtgaaatccggcatccatcgtacggttcacgcaggtgaggtcggctcagccgaagtggttaaagaggcggtggatattctgaaaaccgaacgcctcggccacggttaccacaccttggaagaccaagcactgtataaccgtctgcgccaggagaatatgcattttgaaatctgcccctggagcagttacttaactggcgcctggaaacctgacacagaacatgcagtgattcgtttgaaaaacgatcaggcgaattattctcttaacacggatgatccgctgatattcaaatccaccctggataccgactaccagatgaccaaacgcgatatggggtttacagaggaagaatttaaacgtctgaatattaacgcggcgaaatcaagctttttgccagaggatgaaaagcgcgaacttttagatctgctgtataaggcgtacggtatgccgccttcggcaagcgcgggccagaacttgggcggtcatcatcaccaccatcacggcggctaa

SEQ ID NO: 258 - protein, 600 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGG

SEQ ID NO: 259 - DNA, 1059 bp, synthetic construct
  source 1..1059: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttgtcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaataa

SEQ ID NO: 260 - protein, 352 aa, synthetic construct
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 261 - DNA, 1059 bp, synthetic construct
  source 1..1059: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttctcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaataa

SEQ ID NO: 262 - protein, 352 aa, synthetic construct
MAQTPAFNKPKVELHVHLDGAIKPETILYFGKKRGIALPADTVEELRNIIGMDKPLSLPGFLAKFDYYMPVIAGSREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 263 - DNA, 1089 bp, synthetic construct
  source 1..1089: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttctcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaaggcggtcatcatcaccaccatcacggcggctaa

SEQ ID NO: 264 - protein, 362 aa, synthetic construct
MAQTPAFNKPKVELHVHLDGAIKPETILYFGKKRGIALPADTVEELRNIIGMDKPLSLPGFLAKFDYYMPVIAGSREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQGGHHHHHHGG

SEQ ID NO: 265 - DNA, 1089 bp, synthetic construct
  source 1..1089: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttgtcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaaggcggtcatcatcaccaccatcacggcggctaa

SEQ ID NO: 266 - protein, 362 aa, synthetic construct
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQGGHHHHHHGG

SEQ ID NO: 267 - DNA, 1125 bp, synthetic construct
  source 1..1125: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttgtcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaaggcggtcatcatcaccaccatcacggcggccatcaccatcaccatcaccatcaccatcacgggggttaa

SEQ ID NO: 268 - protein, 374 aa, synthetic construct
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQGGHHHHHHGGHHHHHHHHHHGG

SEQ ID NO: 269 - DNA, 1113 bp, synthetic construct
  source 1..1113: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttgtcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaaggcggtcatcatcaccaccatcacggcggccatcaccatcaccatcacgggggttaa

SEQ ID NO: 270 - protein, 370 aa, synthetic construct
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQGGHHHHHHGGHHHHHHGG

SEQ ID NO: 271 - DNA, 1146 bp, synthetic construct
  source 1..1146: mol_type = other DNA
atggcgcagacgccggcattcaataagccaaaagtcgagttgcacgtacatctggacggggccataaaaccggaaactatcttatactttggaaaaaagcgtggtattgcgctgcccgcagataccgttgaggaacttcgcaacatcattggcatggataaaccgctgtcccttcctgggtttttagcaaaattcgattactatatgccggtgatcgctggttgtcgtgaagcgattaaacgcattgcctacgagtttgttgaaatgaaggcaaaagaaggtgtggtctatgtagaggttcgttattcgccacacctgcttgccaattctaaagtggatcctatgccgtggaaccagaccgaaggcgacgttacaccagatgatgtcgtggacctggtaaatcaaggtttgcaggaaggcgaacaggcgttcggaattaaagttcgcagcattctgtgctgtatgcgtcatcaaccgtcatggagtcttgaagtgctggagctttgcaaaaagtacaaccagaaaacggttgtggctatggatctggccggcgatgaaactatcgaaggtagctctttatttccaggccacgtcgaagcgtatgagggcgctgtaaaaaatggtattcatcgcaccgtgcatgcgggcgaagtgggtagtccggaagtcgtacgtgaggccgttgacatcctgaaaacggaacgcgtggggcatggctatcacaccattgaagatgaagcactgtacaaccgtctcctgaaagagaatatgcatttcgaagtgtgcccgtggagctcatatctcaccggtgcctgggatcccaaaacaacgcacgctgttgtccgctttaaaaacgacaaggcgaattatagcttgaacaccgatgacccgctgatatttaaatccactctggatacagattaccagatgacgaagaaagacatgggattcaccgaagaggaatttaaacgtctcaatatcaacgcagcgaaatcgtcttttctgcctgaagaggaaaagaaagaactgttagagcgcttgtatcgcgaatatcaagagaacctgtacttccaatccggcggtcatcatcaccaccatcacggcggccatcaccatcaccatcaccatcaccatcacgggggttaa

SEQ ID NO: 272 - protein, 381 aa, synthetic construct
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQENLYFQSGGHHHHHHGGHHHHHHHHHHGG

SEQ ID NO: 273 - DNA, 69 bp, synthetic construct
  source 1..69: mol_type = other DNA
atgcggcctacttgggcctggtggctgttcctggtgctgctgctagctctgtgggcacccgctagaggg

SEQ ID NO: 274 - protein, 23 aa, synthetic construct
MRPTWAWWLFLVLLLALWAPARG

SEQ ID NO: 275 - DNA, 30 bp, synthetic construct
  source 1..30: mol_type = other DNA
ggaggaggtggctctggcggtggaggttcc

SEQ ID NO: 276 - protein, 10 aa, synthetic construct
GGGGSGGGGS

SEQ ID NO: 277 - DNA, 30 bp, synthetic construct
  source 1..30: mol_type = other DNA
ggcggacaccaccatcaccaccatggaggc

SEQ ID NO: 278 - protein, 10 aa, synthetic construct
GGHHHHHHGG

SEQ ID NO: 279 - DNA, 1088 bp, synthetic construct
  source 1..1088: mol_type = other DNA
atggcccagacgcccgccttcgacaagcccaaagtggaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacct

SEQ ID NO: 280 - protein, 361 aa, synthetic construct
AQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQN

SEQ ID NO: 281 - DNA, 675 bp, synthetic construct
  source 1..675: mol_type = other DNA
actcacacatccccaccgagcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccaaacccccatcccgggatgagctgaccaagaaccaggtcagcctgtcctgcctggtcaaaggcttctatcccagcgacatcgccgtggagtgggagagcaatgggcagccggagaacaactacaagaccacggtccccgtgctggactccgacggctccttcaggctcgccagctatctcaccgtggacaagagcaggtggcagcaggggaacgtcttctcatgctccgtgatgcatgaggctctgcacaaccactacacgcagaagagcctctccctgtctccgggtaaa

SEQ ID NO: 282 - protein, 225 aa, synthetic construct
THTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 283 - DNA, 1890 bp, synthetic construct
  source 1..1890: mol_type = other DNA
atgcggcctacttgggcctggtggctgttcctggtgctgctgctagctctgtgggcacccgctagaggggcccagacgcccgccttcgacaagcccaaagtggaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggaggaggtggctctggcggtggaggttccactcacacatccccaccgagcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccaaacccccatcccgggatgagctgaccaagaaccaggtcagcctgtcctgcctggtcaaaggcttctatcccagcgacatcgccgtggagtgggagagcaatgggcagccggagaacaactacaagaccacggtccccgtgctggactccgacggctccttcaggctcgccagctatctcaccgtggacaagagcaggtggcagcaggggaacgtcttctcatgctccgtgatgcatgaggctctgcacaaccactacacgcagaagagcctctccctgtctccgggtaaaggcggacaccaccatcaccaccatggaggc

SEQ ID NO: 284 - protein, 630 aa, synthetic construct
MRPTWAWWLFLVLLLALWAPARGAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGGGSGGGGSTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGHHHHHHGG

SEQ ID NO: 285 - DNA, 1890 bp, synthetic construct
  source 1..1890: mol_type = other DNA
atgcggcctacttgggcctggtggctgttcctggtgctgctgctagctctgtgggcacccgctagagggactcacacatccccaccgagcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccaaacccccatcccgggatgagctgaccaagaaccaggtcagcctgtcctgcctggtcaaaggcttctatcccagcgacatcgccgtggagtgggagagcaatgggcagccggagaacaactacaagaccacggtccccgtgctggactccgacggctccttcaggctcgccagctatctcaccgtggacaagagcaggtggcagcaggggaacgtcttctcatgctccgtgatgcatgaggctctgcacaaccactacacgcagaagagcctctccctgtctccgggtaaaggaggaggtggctctggcggtggaggttccgcccagacgcccgccttcgacaagcccaaagtggaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctcggcggacaccaccatcaccaccatggaggc

SEQ ID NO: 286 - protein, 630 aa, synthetic construct
MRPTWAWWLFLVLLLALWAPARGTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKGGGGSGGGGSAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGGHHHHHHGG

SEQ ID NO: 287 - DNA, 46 bp, synthetic construct
  source 1..46: mol_type = other DNA
aactttaagaaggagatataccatggctcaaactccggccttcgac

SEQ ID NO: 288 - DNA, 40 bp, synthetic construct
  source 1..40: mol_type = other DNA
tggtggtgatgatgaccgcccaagttctggcccgcgcttg

SEQ ID NO: 289 - DNA, 58 bp, synthetic construct
  source 1..58: mol_type = other DNA
cgagtgcggccgcaagcttgtcgacttagccgccgtgatggtggtgatgatgaccgcc

SEQ ID NO: 290 - protein, 373 aa, synthetic construct; identical to SEQ ID NO: 2

SEQ ID NO: 291 - DNA, 27 bp, synthetic construct
  source 1..27: mol_type = other DNA
ggaattgtgagcggataacaattcccc

SEQ ID NO: 292 - DNA, 37 bp, synthetic construct
  source 1..37: mol_type = other DNA
cagtggtggtggtggtggtggccgccgtgatggtggt

SEQ ID NO: 293 - protein, 16 aa, synthetic construct
GGHHHHHHGGHHHHHH

SEQ ID NO: 294 - DNA, 31 bp, synthetic construct
  source 1..31: mol_type = other DNA
ggggaattgtgagcggataacaattcccctc

SEQ ID NO: 295 - DNA, 71 bp, synthetic construct
  source 1..71: mol_type = other DNA
gccgccgtgatggtggtgatgatgaccgccggattggaagtacaggttctccaagttctggcccgcgcttg

SEQ ID NO: 296 - DNA, 24 bp, synthetic construct
  source 1..24: mol_type = other DNA
ggcggtcatcatcaccaccatcac

SEQ ID NO: 297 - DNA, 20 bp, synthetic construct
  source 1..20: mol_type = other DNA
actgcgatccccgggaaaac

SEQ ID NO: 298 - DNA, 20 bp, synthetic construct
  source 1..20: mol_type = other DNA
cttctaatacctggaatgct

SEQ ID NO: 299 - DNA, 25 bp, synthetic construct
  source 1..25: mol_type = other DNA
ggtatatctccttcttaaagttaaa

SEQ ID NO: 300 - protein, 10 aa, synthetic construct
GGHHHHHHGG

SEQ ID NO: 301 - DNA, 59 bp, synthetic construct
  source 1..59: mol_type = other DNA
tcgagtgcggccgcaagcttgtcgacttagccgccgtgatggtggtgatgatgaccgcc

(19) United States
(12) Patent Application Publication
(10) Pub. No.: US 2025/0075196 A1
(43) Pub. Date: Mar. 6, 2025

(54) ADENOSINE DEAMINASE 1 COMPOSITIONS AND METHODS FOR USING SAME

(71) Applicant: Georgia Tech Research Corporation, Atlanta, GA (US)

(72) Inventors: John James Blazeck, Atlanta, GA (US); Maria Rain Jennings, Atlanta, GA (US)

(21) Appl. No.: 18/708,766

(22) PCT Filed: Nov. 11, 2022
(86) PCT No.: PCT/US2022/079705

§ 371 Date: May 9, 2024

Related U.S. Application Data
(60) Provisional application No. 63/278,777, filed on Nov. 12, 2021.

(51) Int. Cl. C12N 9/78

(52) U.S. Cl. CPC C12N 9/78; C12Y 305/04004

(57) ABSTRACT

The invention is directed to mutants of adenosine deaminase 1 (ADA1) that have increased or prolonged stability or catalytic activity relative to wildtype ADA1, as well as compositions comprising the mutants and methods of using the mutants to treat various conditions.

### CROSS-REFERENCE TO RELATED APPLICATIONS

This application claims the benefit of U.S. Provisional Application Ser. No. 63/278,777, filed on 12 Nov. 2021, which is incorporated herein by reference in its entirety as if fully set forth below.

### FEDERALLY SPONSORED RESEARCH STATEMENT

This invention was made with government support under grant/award number GR00003567 awarded by the National Science Foundation and grant/award numbers GR00013867 and GR00017721 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Jan. 12, 2023, is named GTRC8850PCT_SL.xml and is 475,116 bytes in size.

### FIELD OF THE DISCLOSURE

The various embodiments of the present disclosure relate generally to mutants of human adenosine deaminase 1 (HsADA1) and more particularly to compositions comprising the mutated HsADA1 enzymes and methods of using the compositions to treat cancer, tumors, and Adenosine Deaminase Severe Combined Immune Deficiency (ADA-SCID). In some embodiments, the mutations are located in certain conserved areas of the protein and alter the charge of the amino acids at specific positions, thus conferring increased or prolonged stability or catalytic activity under physiological conditions (e.g., 37° C. and pH 7.4) relative to wildtype HsADA1.

### BACKGROUND

Adenosine is a molecule that is accumulates in human solid tumors by cancer cells and immunosuppressive immune cells alike. Adenosine has been shown to prevent immune cells, particularly T cells, from activating and subsequently killing cancerous cells. Immunotherapies stop cancers from engaging or re-engaging pathways in immune cells that have been shut off by tumoral mechanisms, thereby encouraging robust anti-cancer immune cell responses. Immunotherapies have shown clinical success against several cancer types, including melanoma, non-small-cell lung cancer, and renal-cell cancer. These promising results represent a new paradigm in cancer treatment, but, given the complex network of interactions that regulate the functionality of immune cells, cancers can engage numerous distinct pathways to thwart an immune response. It is widely accepted that modulation of multiple immune regulatory pathways is required. In this regard, extensive studies have established the potent immunosuppressive effects of the purine ribonucleoside adenosine (ADO). There are many distinct enzymes and metabolic pathways that produce adenosine in tumors, where it accumulates to levels approaching 1000 times higher than in normal tissue. ADO accumulation in the tumor microenvironment induces tolerance and inhibits anti-cancer immune responses. ADO can activate any of the four extracellular G protein coupled adenosine receptors (Ars), AiAR, A2AAR, A2BAR, and A3AR, to mediate effects in a cell type dependent manner. The high affinity A2AAR and the lower affinity A2BAR are major players in inhibiting anticancer immune responses. Particularly, ADO agonism of ARs modulates the function of numerous adaptive and innate immune cells to reduce the overall immune response. As such, the pharmacological blockade of ADO accumulation and impact is of great interest and has driven the discovery of multiple small molecules that antagonize A2AAR and monoclonal antibodies and inhibitors that target CD39 and CD73. There are three small molecule antagonists of A2AAR and four CD73-inhibiting antibodies in seventeen total phase I/II clinical evaluations, and a CD39 inhibitor and aCD39 antibody are in preclinical development. CD73 and CD39 are two of many enzymes that are involved in ADO synthesis by tumors.

Current efforts towards the pharmacological inhibition of ADO synthesis or A2AAR agonism using small molecule inhibitors or antibodies suffer from important shortcomings: First, several enzymes can produce ADO. Therefore, targeting CD39/CD73 axis to limit ADO synthesis is limited by enzymatic redundancies. Second, ADO can bind to and elicit effects through three ADO receptors apart from A2AAR. Thus, single receptor targeting is highly unlikely to reverse all mechanisms of ADO-induced suppression.

There is therefore a need for an improved composition and/or method of inhibiting ADO's effect on the immune system that can target all mechanisms of ADO-induced lsuppression. It is to such a composition and method that embodiments of the invention are directed.

### BRIEF SUMMARY

The present disclosure relates to mutants of human (Homo sapiens) adenosine deaminase 1 (HsADA1) and more particularly to compositions comprising the mutated HsADA1 enzymes and methods of using the compositions to treat cancer, tumors, and Adenosine Deaminase Severe Combined Immune Deficiency (ADA-SCID). In some embodiments, the mutations are located in certain conserved areas of the protein and alter the charge of the amino acids at specific positions, thus conferring prolonged catalytic stability under physiological conditions (e.g., 37° C. and pH 7.4) and in biological fluids (e.g., serum) relative to wildtype HsADA1.

In one aspect, the present invention provides a nucleic acid molecule encoding human adenosine deaminase 1 (HsADA1), wherein the HsADA1 has been mutated to have increased or prolonged stability or catalytic activity at physiological conditions relative to a wildtype HsADA 1.

In another aspect, the present invention provides an amino acid sequence comprising at least one mutation at one or more amino acid positions of human adenosine deaminase 1 (HsADA1) enzyme, wherein the mutated HsADA1 has increased or prolonged stability or catalytic activity at physiological conditions relative to a wildtype HsADA1, and wherein the HsADA1 has been mutated at one or more amino acid positions comprising a charged amino acid to a neutral or differently charged amino acid.

In another aspect, the present invention provides a composition comprising an amino acid sequence comprising at least one mutation at one or more amino acid positions of human adenosine deaminase 1 (HsADA1) enzyme, wherein the mutated HsADA1 has increased or prolonged stability or catalytic activity at physiological conditions relative to a wildtype HsADA1, and wherein the HsADA1 has been mutated at one or more amino acid positions comprising a charged amino acid to a neutral or differently charged amino acid.

In another aspect, the present invention provides a pharmaceutical composition comprising a therapeutically effective amount of an amino acid sequence comprising at least one mutation at one or more amino acid positions of a human adenosine deaminase 1 (HsADA1) enzyme, wherein the mutated HsADA1 has increased or prolonged stability or catalytic activity at physiological conditions relative to a wildtype HsADA1, and wherein the HsADA1 has been mutated at one or more amino acid positions comprising a charged amino acid to a neutral or differently charged amino acid.

In another aspect, the present invention provides a method of producing cells configured to express a human adenosine deaminase 1 (HsADA1) polypeptide, the method comprising: introducing a nucleic acid molecule encoding the HsADA1 polypeptide on a vector and operably connected to an inducible promoter into the cells; and culturing the cells under conditions suitable for expression of the HsADA1 polypeptide, wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

In another aspect, the present invention provides a method of treating a cancer or tumor in a subject in need thereof, the method comprising: administering to the subject a therapeutically effective amount of a composition comprising a human adenosine deaminase 1 (HsADA1) polypeptide, wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

In another aspect, the present invention provides a method of treating adenosine deaminase-deficient severe combined immunodeficiency (ADA-SCID) in a subject in need thereof, the method comprising: administering to the subject a therapeutically effective amount of a composition comprising a human adenosine deaminase 1 (HsADA1) polypeptide, wherein the HsADAl polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a plot showing fraction of original activity of Bos taurus ADA (BtADA) and HsADA1 over time, in accordance with an exemplary embodiment of the present invention.

FIG. 1B provides a plot showing fraction of original activity of BtADA and HsADA1 over time, in accordance with an exemplary embodiment of the present invention.

FIG. 2A provides a plot showing fraction of original activity of BtADA, K164E L194F, K164E, and HsADA1 over time, in accordance with an exemplary embodiment of the present invention.

FIG. 2B provides a plot showing fraction of original activity of BtADA, K164E L194F Q173N, K164E L194F Q202E, K164E L194F, K164E, and HsADA1 over time, in accordance with an exemplary embodiment of the present invention.

FIG. 3A provides a plot showing fraction of original activity of BtADA, K164E L194F, K164E, and HsADA1 over time, in accordance with an exemplary embodiment of the present invention.

FIG. 3B provides a plot showing fraction of original activity of BtADA, K164E L194F, K164E, and HsADA1 over time, in accordance with an exemplary embodiment of the present invention.

FIG. 4A provides a plot showing fraction of original activity of K164E L194F Q173N, K164E L194F, K164E, and HsADA1 over time, in accordance with an exemplary embodiment of the present invention.

FIG. 4B provides a plot showing fraction of original activity of K164E L194F Q202E, K164E L194F, K164E, and HsADA1 over time, in accordance with an exemplary embodiment of the present invention.

FIG. 5A provides a plot showing fraction of original activity of mutants disclosed herein and HsADA1 over time, in accordance with an exemplary embodiment of the present invention.

FIG. 5B provides a plot showing fraction of original activity of mutants disclosed herein and HsADA1 over time, in accordance with an exemplary embodiment of the present invention.

FIG. 6A provides a plot showing fraction of original activity of mutants disclosed herein and HsADA1 over time, in accordance with an exemplary embodiment of the present invention.

FIG. 6B provides a plot showing fraction of original activity of mutants disclosed herein and HsADA1 over time, in accordance with an exemplary embodiment of the present invention.

FIG. 7 provides a plot showing fraction of original activity of Escherichia coli lysate containing mutants disclosed herein and HsADA1 over time, in accordance with an exemplary embodiment of the present invention.

FIG. 8 provides a plot showing fraction of original activity of Escherichia coli lysate containing mutants disclosed herein and HsADA1 over time, in accordance with an exemplary embodiment of the present invention.

FIG. 9 provides a plot showing fraction of original activity of Escherichia coli lysate containing mutants disclosed herein and HsADA1 over time, in accordance with an exemplary embodiment of the present invention.

FIG. 10 provides a diagrammatic representation of a HsADA1 protein fusion that can improve its in vivo residence time, such as an IgG-Fc-HsADA1 fusion protein, or its purification, in accordance with an exemplary embodiment of the present invention.

FIG. 11A provides an illustration of protein-protein interactions between a T cell and a cancer cell, in accordance with an exemplary embodiment of the present invention.

FIG. 11B provides an illustration of small molecule signals in a biological system, in accordance with an exemplary embodiment of the present invention.

FIG. 12A provides a plot of Anti-CTLA-4 response versus adenosine signaling, in accordance with an exemplary embodiment of the present invention sourced from Sidders, Ben, et al. “Adenosine Signaling Is Prognostic for Cancer Outcome and Has Predictive Utility for Immunotherapeutic Response, The Landscape of Adenosine Signaling in Cancer.” Clinical Cancer Research 26.9 (2020): 2176-2187.

FIG. 12B provides a plot of Anti-PD1 response versus adenosine signaling, in accordance with an exemplary embodiment of the present invention sourced from Sidders, Ben, et al. “Adenosine Signaling Is Prognostic for Cancer Outcome and Has Predictive Utility for Immunotherapeutic Response, The Landscape of Adenosine Signaling in Cancer.” Clinical Cancer Research 26.9 (2020): 2176-2187.

FIG. 13A shows catalytic efficiency of HsADA1, in accordance with an exemplary embodiment of the present invention.

FIG. 13B shows activity of HsADA1 across a pH range, in accordance with an exemplary embodiment of the present invention.

FIG. 14 provides data showing increased yield of HsADA1 due to process optimization and mutagenesis, in accordance with an exemplary embodiment of the present invention.

FIG. 15A provides data showing increased survival of mice with colorectal cancer tumors in terms of tumor size versus time, in accordance with an exemplary embodiment of the present invention.

FIG. 15B provides data showing increased survival of mice with colorectal cancer tumors in terms of tumor size versus time, in accordance with an exemplary embodiment of the present invention.

FIG. 16 provides a diagrammatic representation of redundancy of adenosine generation mechanisms, in accordance with an exemplary embodiment of the present invention.

FIG. 17 provides a diagrammatic representation of tumors mediating their own progression and thwarting immune responses through complex, redundant signaling networks, in accordance with an exemplary embodiment of the present invention.

FIG. 18 provides data related to Michaelis-Menten kinetics, in accordance with an exemplary embodiment of the present invention.

FIG. 19 provides SDS-PAGE analysis of HsADA1 before and after cleavage of the His-tag with TEV protease showing expected decrease in molecular mass, in accordance with an exemplary embodiment of the present invention.

FIG. 20A provides a size exclusion chromatography elution profile for cleaved HsADA1, in accordance with an exemplary embodiment of the present invention.

FIG. 20B provides Michaelis-Menten kinetic parameters of cleaved HsADA1 at pH 7.4, in accordance with an exemplary embodiment of the present invention.

FIG. 21A provides data related to the stability of tagged and cleaved HsADA1 obtained from differential scanning fluorimetry, in accordance with an exemplary embodiment of the present invention.

FIG. 21B provides data related to His-tagged HsADA1 activity as a function of pH condition, in accordance with an exemplary embodiment of the present invention.

FIG. 22A provides data related to activity remaining for 11 single site amino acid mutations of HsADA1, in accordance with an exemplary embodiment of the present invention.

FIG. 22B provides data related to percent activity of purified variants of HsADA1 with improved retention of activity at 37 degrees Celsius, in accordance with an exemplary embodiment of the present invention.

FIG. 23 provides a schematic of the therapeutic value of adenosine degradation, in accordance with an exemplary embodiment of the present invention.

FIG. 24A provides data related to kinetics for HsADA1 mutants, in accordance with an exemplary embodiment of the present invention.

FIG. 24B provides data related to kinetics for HsADA1 mutants, in accordance with an exemplary embodiment of the present invention.

FIG. 25A provides data related to kinetics for HsADA1 mutants, in accordance with an exemplary embodiment of the present invention.

FIG. 25B provides data related to kinetics for HsADA1 mutants, in accordance with an exemplary embodiment of the present invention.

FIG. 26 provides data related to kinetics for HsADA1 mutants, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint(s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a composition comprising a mutated ADA1 and at least a second pharmaceutically active ingredient means at least two, but any desired combination of compounds can be delivered simultaneously or sequentially (e.g., within a 24-hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

### Compositions of the Invention

According to some aspects, the present invention provides mutated HsADA1 enzymes and nucleic acids encoding those enzymes, as well as compositions comprising the mutated HsADA1 enzymes, preferably present in therapeutically effective amounts.

Regarding regions and residues engineered for HsADA1, as opposed to its Bos taurus (Bt) and Mus musculus (Mm) homologs, Homo sapiens adenosine deaminase isoform 1 (HsADA1) loses its enzymatic activity within hours at physiological conditions, i.e., in human serum at 37 degrees Celsius and pH 7.4. BtADA and MmADA retain full activity over the course of a week at the same conditions, i.e., they are stable in physiological conditions. To recapitulate the stability of Bt/MmADA1 in HsADA1, the inventors introduced to HsADA1 mutations at residues whose identity differed from that of the Bt and Mm homologs. In most instances, the inventors mutated the residue identity to that of Bt/Mm, and in general, mutating charged residues within domains of interest to either neutralize or reverse the charge at that location improved stability in physiological conditions in the resulting HsADA1 variant.

The residues described further herein include D8, S21, R33, N41, G45, V49, T57, G134, A148, N160, P163, K164, Q173, Q174, P189, L194, Q199, Q202, 1261, 1281, L283, and R341. All positions described herein are relative to wildtype HsADA1 found in SEQ ID NO: 279. The mutated ADA1 enzymes can comprise more than one mutation in these positions; for example, a mutated HsADA1 enzyme can comprise two mutations or three mutations in these positions. Preferred positions include D8, S21, R33, N41, G45, T57, G134, A148, N160, P163, K164, Q173, P189, L194, Q199, Q202, 1261, 1281, L283, and/or R341.

Preferred substitutions include N at position D8; A at position S21; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.

In some embodiments, the HsADA1 variant includes at least one mutation in R33K, N41D, K164E, Q173N, L194F, Q199K, and/or Q202E.

In some embodiments, the HsADA1 variant includes three mutations, which can be located in positions (i) K164, L194, and Q199; (ii) K164, L194, and Q173; or (iii) K164, L194, and Q202. In some embodiments, the HsADA1 mutant can comprise the following mutations: (i) K164E, L194F, and Q199K; (ii) K164E, L194F, and Q173N; or (iii) K164E, L194F, and Q202E.

Based on these residues and their place in the HsADA1, it is suggested that mutating residues with the linear amino acid sequence of N160 to Q202 (inclusive) can increase HsADA1 stability.

Based on the importance of increasing homology to Bt- and/or MmADA, it is suggested that mutating the residues Y30-->F, R32-->K, D60-->E or G, A71-->V, E77-->D, I79-->V, E113-->D, I115-->M, Al20-->T, L124-->V, E128-->D, A131-->D or S, R142-->Q, D143-->A, V146-->I, V166-->L, Q175-->K, I180-->M, E203-->G, S207-->N, I209-->V, A221-->P, E222-->N, K225-->R, I230-->T, L236-->V, L243-->I, Q246-->E or T, A247-->T, R253-->L, Q254-->K, K273-->D, D275-->K, E277-->T, A279-->P, Q287-->K, A288-->V, R313-->K or N, D314-->E, D338-->E, D345-->E, L346-->R, K349-->R, A350-->E, and G352-->Q could also be mutated in some combination to prolong HsADA1 enzymatic activity/stability at physiological conditions. These positions can be mutated in combination with any mutation at positions D8, S21, R33, N41, G45, V49, T57, G134, A148, N160, P163, K164, Q173, Q174, P189, L194, Q199, Q202, 1261, 1281, L283, and/or R341.

In an aspect, the invention provides a nucleic acid molecule encoding human adenosine deaminase 1 (HsADA1), wherein the HsADA1 has been mutated to have increased or prolonged stability or catalytic activity at physiological conditions relative to a wildtype HsADA1, and that result in an alteration in the charged amino acid (e.g., replacing with a neutral amino acid or an oppositely charged amino acid). The mutations can be any of the mutations described herein. For example, the HsADA1 nucleic acid includes one or more mutations at positions D8, S21, R33, N41, G45, V49, T57, G134, A148, N160, P163, K164, Q173, Q174, P189, L194, Q199, Q202, 1261, 1281, L283, and/or R341, optionally in combination with one or more of the following mutations: Y30-->F, R32-->K, D60-->E or G, A71-->V, E77-->D, I79-->V, E113-->D, I115-->M, Al20-->T, L124-->V, E128-->D, A131-->D or S, R142-->Q, D143-->A, V146-->I, V166-->L, Q175-->K, I180-->M, E203-->G, S207-->N, I209-->V, A221-->P, E222-->N,

K225-->R, I230-->T, L236-->V, L243-->I, Q246-->E or T, A247-->T, R253-->L, Q254-->K, K273-->D, D275-->K, E277-->T, A279-¬>P, Q287-->K, A288-->V, R313-->K or N, D314-->E, D338-->E, D345-->E, L346-->R, K349-->R, A350-->E, and G352-->Q. The nucleic acid molecule can comprise a nucleotide sequence as set forth in SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59,

61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 37, 139, 141, 143, 145, 147, 149, 151, 153, 155, 157, 159, 161, 163, 165, 167, 169, 171, 173, 175, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 283, and 285.

The nucleic acid molecule encoding the HsADA1 variant can be present in a vector (e.g., a viral vector or a plasmid) or in an expression cassette. The vector or expression cassette can be present in a cell, such as a prokaryotic cell or eukaryotic cell. The vector or expression cassette may be permanently or transiently integrated into the host cell genome, or may be maintained extrachromasomally by suitable methods such as selection pressure. The vector or expression cassette can include a strong promoter operably linked to the nucleic acid molecule encoding the HsADA1 variant. The promoter can be inducible or constitutive. Nonlimiting examples of prokaryotic cells include cells suitable for expression of heterologous proteins, such as for example and not limitation, Escherichia coli. Nonlimiting examples of eukaryotic cells include mammalian cells, such as immune cells and non-immune cells. Nonlimiting examples of immune cells include T cells, CAR T cells, B cells, natural killer (NK) cells, and neutrophils. Nonlimiting examples of non-immune cells can include cell lines that are suitable for expression of heterologous proteins, such as for example and not limitation, Chinese hamster ovary (CHO) cells, HEK293T Cells, and Expi293T cells.

In another aspect, the present invention provides an HsADA1 polypeptide with one or more mutations that confer increased or prolonged stability or catalytic activity at physiological conditions (e.g., 37° C. and pH 7.4) and in biological fluids (e.g., serum) relative to a wildtype HsADA1, and that result in an alteration in the charged amino acid (e.g., replacing with a neutral amino acid or an oppositely charged amino acid). The mutations can be any of the mutations described herein. For example, the HsADA1 nucleic acid includes one or more mutations at positions D8, S21, R33, N41, G45, V49, T57, G134, A148, N160, P163, K164, Q173, Q174, P189, L194, Q199, Q202, 1261, 1281, L283, and/or R341, optionally in combination with one or more of the following mutations: Y30-->F, R32-->K, D60-->E or G, A71-->V, E77-->D, I79-->V, E113-->D, I115-->M, Al20-->T, L124-->V, E128-->D, A131-->D or S, R142-->Q, D143-->A, V146-->I, V166-->L, Q175-->K, 1180-->M, E203-->G, S207-->N, I209-->V, A221-->P, E222-->N, K225-->R, I230-->T, L236-->V, L243-->I, Q246-->E or T, A247-->T, R253-->L, Q254-->K, K273-->D, D275-->K, E277-->T, A279-->P, Q287-->K, A288-->V, R313-->K or N, D314-->E, D338-->E, D345-->E, L346-->R, K349-->R, A350-->E, and G352-->Q. The nucleic acid molecule can comprise a nucleotide sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

In an embodiment, the mutated HsADA1 polypeptide can be operably linked to portions of an IgG molecule. For example and not limitation, the signal portion of IgG (e.g., as recited in SEQ ID NOs: 273-274) can be operably fused to the N-terminus of the HsADA1 polypeptide, optionally in combination with a linker. An exemplary linker is a glycine-serine linker such as that recited in SEQ ID NOs: 275-276. The Fc portion of the IgG antibody can be operably fused to the C-terminus of the HsADA1 polypeptide (e.g., as recited in SEQ ID NOs: 281-282). It is contemplated herein that the Fc portion is an engineered Fc portion that has been optimized for expression in a heterologous host cell, e.g., Escherichia coli.

In an embodiment, the mutated HsADA1 polypeptide can be modified to increase its half-life, solubility, and/or ability to interact with certain cancers and/or tumors (e.g., solid tumors by way of adding a domain that binds to or interacts with collagen). In an embodiment, the mutated HsADA1 polypeptide can be operably linked to an antibody fragment, such as for example and not limitation, an Fc portion, to create a peptibody. In an embodiment, the HsADA1 polypeptide can be PEGylated at its N-terminus and/or C-terminus. The mutated HsADA1 polypeptide can also be operably linked to an scFv portion of an antibody. In an example, the scFv portion can be from a collagen-associated antibody. The mutated HsADA1 polypeptide can be operably linked to a collagen-binding peptide. All of these modifications are encompassed within the term “HsADA1 variants”.

In another aspect, the invention provides a composition comprising one or more HsADA1 variants as described herein (referred to herein as ADA1-containing compositions). The HsADA1 variant is present in a therapeutically effective amount. The HsADA1-containing composition can further comprise additional ingredients, such as for example and not limitation, a pharmaceutically acceptable excipient and/or carrier.

The HsADA1-containing composition can be formulated for administration by any appropriate route, such as for example and not limitation, intratumoral, peritumoral, intradermal, subcutaneous, intravenous, or intraperitoneal administration. Suitable excipients and/or carriers can be chosen based on the route of administration.

### Combination Therapies

Any of the HsADA1-containing compositions (polypeptides or nucleic acids) disclosed herein can be used in combination with immunotherapy regimens, a non-limiting list of which is provided herein. The HsADA1-containing compositions can either be co-administered with engineered T cells or the engineered T cells could be engineered to simultaneously express and secrete these proteins. Adoptive cell therapies contemplated herein are adoptive T cell Therapy with tumor-infiltrating lymphocytes and Chimeric antigen receptor T cells (CAR-T) therapies including: aCD19, aBCMA, aSLAMF7, (CS1), aNKG2D, aCD56, aCD70, aCD38, aCD138, aCD44v6, aCD123, aCD33, aLewis Y, aFLT-3, aCLL-1, aILl-RAP, aTIM3, aCD19/aCD22, aCD19/aCD20, aCD33/aCD123, aBCMA/aCD38, aBCMA/TACI, aBCMA/CD19, aBCMA/CS1, aEpCAM/aICAM-1, and aCAIX/aCD70.

Chimeric antigen receptor natural killer cell (CAR-NK) therapies can include Sipuleucel-T.

Antibody therapies useful with the ADA1-containing compositions can include aPD-1, aPD-L1 (B7-H1), aCTLA-4, aLAG3 (CD223), aTIM3, aHER-2, aCD52, aVEGF, aVEGFR, aVEGFR2, aEGFR, aRANKL, aGD2, aSLAMF7, aCD38, aCCR4 (CD194), aCD20, aPDGFR-a, aCD19, aB7-H3 (CD276), aCD47, aSIRPa, a4-1BB CD137), aICOS (CD278), aCD70, aGITR (CD357), aOX40 (CD134), aCD40, aVISTA (B7-H5), aIDO1, aIDO2, aTIGIT, aA2AAR, aTGFbeta, aCD73, aCD27, and aKIR (2DL1-3).

Antibody-Drug conjugates can include aBCMA/mafodotin-blmf, aCD30/vedotin, aNectin-4/vedotin, aCD33/ozogamicin, aCD20/tiuxetan, aCD22/ozogamicin, aCD22/pasudotox, aCD19/tesirine, aCD79b/vedotin, aTROP-2/govitecan-hziy, aTF/vedotin, aHER2/deruxtecan, aHER2/emtansine, and aCD30/monomethyl auristatin E.

Bispecific antibodies useful with the ADA1-containing compositions can include aCD3 e/CD 19, aCD3 e/CD20, aCD3 e/CD33, aCD3e/FLT3, aCD3e/HER2, aCD3e/PSMA, aCD3e/EGFRvIII, aCD3e/DLL3, aCD3 e/MUC17, aCD3e/CLDN18.2, aCD3/B7-H3, aEGFR/MET, aCD3/gp100, aPD-L1/4-1BB, aPD-1/PD-L1, aPD-1/LAG3, aCD30/CD16A, aCD3/CD38, aCD19/CD47, aCD47/CD20, aCD37/CD3, aCLEC12A/CD3, aPD-1/CTLA-4, aHer2/Her2, aCEA/CD3, aEpCAM/CD3, aIGF-1/IGF-2, aVEGF/Ang2, aEGFR/cET, aDLL4/VEGF, aHER2/HER3, aPD-Ll/CD137, aPSMA/CD3, aIGF-IR/HER3, aPMEL/CD3, aGPA33/CD3, aGPC3/CD3, aPD-1/Tim3, aPD-Ll/Tim3, aCD16/CD30, aCD16/CD33, aCD3/p-cadherin, aPD-1/ICOS, aHSA/TNF, aHSA/IL6R, aHSA/IL17A/F, aHSA/RANKL, aIL-13/IL-4, aIL-13/IL-17, aBAFF/ICOSL, aBAFF/IL-17A, and aNGF/TNF.

Cytokine therapies can include Neo-2/15, IL-2, IL-7, IL-12, IL-15 (or adaptations, e.g., ALT-03), IL-18, IL-21, TNFa, GM-CSF, IFNa-2a, IFNa-2b, and IFNy.

Immunomodulatory drugs useful with the ADA1-containing compositions can include Thalidomide, Lenalidomide, Pomalidomide, Imiquimod, Poly ICLC, Pexidartinib (small molecule inhibitor of the KIT, CSF1R, and FLT3 pathways), Propanolol, Atenolol, Beta-blockers, Aspirin, Celecoxib, Rofecoxib, and Valdecoxib.

Immunogenic cell death inducers useful with the ADA1-containing compositions can include Doxorubicin, Mitoxantrone, Epirubicin, Bleomycin, Oxaliplatin, Cyclophosphamide, and Bortezomib.

Cancer vaccines useful with the ADA1-containing compositions can include Bacillus Calmette-Guerin.

Oncolytic virus therapies useful with the ADA1-containing compositions can include T-VEC, Adenovirus, Herpes simplex virus, Maraba virus, Measles, Newcastle Disease Virus, Picornavirus, Reovirus, Vaccinia virus, and Vesicular stomatitis virus.

Nano-immunotherapies useful with the ADA1-containing compositions can include Thermosensitive PLGA nanoparticle, Immunoliposome, Poly (propyl acrylic acid) nanoplex, Chitosan Nanoparticle, PEG-b-PAEMA pH-sensitive cluster nanoparticle, High density lipoprotein nanodisc, Lipid nanocapsules, Polyglycerol and Cyclic tripeptides of L-arginine, glycine and L-aspartic acid nanodiamond, Multi-walled carbon nanotubes, Polyethyleneimine nanoplexes, Lipid nanoparticle (DOPE and Cholesterol), Lipoplex (DOTAP and m-PEG-PLA), 1,2-dioleoy1-3-trimethylammonium propane and dioleolylphosphatidyl ethanolamine nanocomplex, Poly (propylene sulfide) and Dextran nanoparticle, DOTAP lipid nanoparticle, Cationic polylactic nanoplexes, PEGylated lipid polyplex, Lipid nanoparticle, Gal-C-dextran nanocomplex, Folic acid-functionalized PEI nanoparticle, and Nanodisc (ApoA-I peptide, Sphingomyelin and Cholesterol).

Therapeutic Enzymes useful with the ADA1-containing compositions can include Arginase-1, Arginase-2, Arginine Deiminase, Asparaginase, Methionase, Lysine oxidase, Phenylalanine ammonia lyase, Glutaminase, Kynureninase, Cytochrome P450, P450 reductase, Herpes virus 1 thymidine kinase (HSV TK), Cytosine deaminase (CD), Nitroreductase, Carboxypeptidase, Horseradish peroxidase, Superoxide dismutase, Carboxylesterase, Glycosidase, Thymidine phosphorylase, Thymidine kinase, Nucleoside phosphorylase, Guanine ribosyltransferase, Phospholipase A2, Glucose oxidase, and Lactate oxidase.

Non-limiting preferred cancer immunotherapies include anti-PD-1 or anti-PD-L1 checkpoint inhibitor antibodies to produce a PD-1 blockade, anti-CTLA-4 checkpoint inhibitor antibodies, anti-LAG3 antibodies, anti-TIM3 antibodies, anti-ICOS antibodies, anti-TIGIT antibodies, anti-GITR antibodies, anti-4-1BB antibodies, anti-0X40 antibodies, anti-CD40 antibodies, anti-CD38 antibodies, anti-B7-H3 antibodies, and/or anti-CD47 antibodies.

The HSADAI variants described herein, optionally in combination with a cancer immunotherapy, adenosine depleting therapy, or ADA1 enzyme replacement therapy as described herein, are useful for treating a condition such as a cancer, a tumor, or ADA-SCID. The HsADA1 variants are present in therapeutically effective amounts, optionally in combination with a pharmaceutically acceptable excipient and/or carrier.

The HsADA1 variants described herein, optionally in combination with one or more ADA1 enzyme replacement therapeutics, are useful for treating ADA-SCID. The HsADA1 variants are present in therapeutically effective amounts, optionally in combination with a pharmaceutically acceptable excipient and/or carrier.

### Methods of Making the HsADA1 Variants

An exemplary method of producing the HsADA1 mutants in a bacterial host follows.

A plasmid containing HsADA1 expressed from a strong IPTG-inducible promoter is transformed into E. coli, and maintained using appropriate selective pressure (e.g., antibiotics). After sufficient growth, the E. coli cultures containing the plasmid are placed in an ice-water slurry and moved to a 4° C. cold room for 30 minutes, followed by addition of 3% v/v ethanol, and each culture is induced to a final concentration of 0.5 mM IPTG. The flasks are then kept for 48-56 hours at 15° C., shaking at 200 RPM, to induce His-tagged HsADA1 protein expression. Induced cultures are harvested by centrifugation at 3,400×g and 4° C. for 30 minutes and stored at −80° C. The E. coli cells can then be lysed and the cell lysates applied to a column capable of trapping His-tagged proteins. The His-tag can be removed by any method known in the art, including a TEV-protease based system as described herein.

A nucleic acid molecule encoding the HsADA1 variant can be present in a vector (e.g., a viral vector or a plasmid) or in an expression cassette. The vector or expression cassette can be present in a cell, such as a prokaryotic cell or eukaryotic cell. The vector or expression cassette may be permanently or transiently integrated into the host cell genome, or may be maintained extrachromosomally by suitable methods such as selection pressure. The vector or expression cassette can include a strong promoter operably linked to the nucleic acid molecule encoding the HsADA1 variant. The promoter can be inducible or constitutive. Nonlimiting examples of prokaryotic cells include cells suitable for expression of heterologous proteins, such as for example and not limitation, Escherichia coli. Nonlimiting examples of eukaryotic cells include mammalian cells, such as immune cells and non-immune cells. Nonlimiting examples of immune cells include T cells, CAR T cells, B cells, natural killer (NK) cells, and neutrophils. Nonlimiting examples of non-immune cells can include cell lines that are suitable for expression of heterologous proteins, such as for example and not limitation, Chinese hamster ovary (CHO) cells, HEK293T Cells, and Expi293T cells.

The cell containing the nucleic acid molecule encoding the HsADA1 variant is cultured under conditions suitable to express the HsADA1 variant, such as for example and not limitation, inducing the expression of the HsADA1 variant, and expanding the population of cells containing the nucleic acid molecule encoding the HsADA1 variant. The cells can be lysed by any appropriate methods, and the HsADA1 variant can be collected or isolated from the cell lysate by any appropriate methods.

### Methods of Treatment Using the HsADA1 Variants

In an aspect, the invention provides a method of treating a disease or condition in a subject in need thereof by administering the HsADA1 variants discussed herein, or compositions comprising therapeutically effective amounts of these variants (referred to herein as HsADA1-containing compositions). The HsADA1 variants or HsADA1-containing compositions can further comprise a pharmaceutically acceptable excipient and/or carrier.

Nonlimiting exemplary diseases and conditions treatable using the HSADA1 variants or the HsADA1-containing compositions include cancers, tumors, and ADA-SCID. Specific non-limiting exemplary cancers and tumors include non-small cell lung cancers, breast cancers including triple negative breast cancers, and colon cancers including unresectable colon cancers with mismatch repair deficiency (CRC-MMR−).

The HsADA1 variants or HsADA1-containing compositions can be administered with one or more cancer immunotherapies or adenosine depleting therapies (if the subject being treated has a cancer or tumor) or with one or more ADA1 enzyme replacement therapeutics (if the subject being treated has ADA-SCID). The HsADA1-containing compositions, or alternatively the HsADA1 variants discussed herein, can be administered by any suitable route, such as for example and not limitation, intratumoral, peritumoral, intradermal, subcutaneous, intravenous, or intraperitoneal administration.

In an embodiment, the HsADA1-containing compositions can be used as a first line treatment. In non-small cell lung cancers with high PD-1 expression (NSCLC-PD-1+), in triple negative breast cancers (TNBC), and in unresectable colon cancers with mismatch repair deficiency (CRC-MMR−), antibody immunotherapies that target PD-1 are now approved as first line therapies. Adenosine has been shown to reduce the efficacy of PD-1 therapies, so it is suggested that the HsADA1-containing compositions described herein can be a first-line therapy in combination with PD-1-targeting antibodies in NSCLC-PD-1+, TNBC, and CRC-MMR-1. TNBC cases account for 13% of the −287,000 breast cancer diagnoses per year and have the poorest outcomes of breast cancer subtypes. NSCLC-PD-1+ account for −28% of the −200,000 non-small cell lung cancer (NSCLC) diagnoses per year, and lung cancers have some of the poorest outcomes of all cancer types. As breast cancers account for 15% of U.S. cancer diagnoses, NSCLC account for 10.5%, and CRC-MMR-account for 1.2%, the HsADA1 compositions described herein can be translated for use as a first line treatment in combination with PD-1 therapies in 6.1% of all US cancer cases.

In an embodiment, the HsADA1-containing compositions can be used as a second line treatment. In addition, as adenosine is implicated as an immunosuppressant across cancer types, the HsADA1 compositions described herein could be efficacious as a monotherapy in potentially any solid tumor type as a second line treatment after failure of chemotherapeutics or radiation therapy.

Nonlimiting exemplary cancer immunotherapies include PD-1 blockade via anti-PD-1 or anti-PD-L1 checkpoint inhibitor antibodies; an anti-CTLA-4 checkpoint inhibitor antibody; an anti-LAG3 antibody; an anti-TIM3 antibody; an anti-ICOS antibody; an anti-TIGIT antibody; an anti-GITR antibody; an anti-4-1BB antibody; an anti-0X40 antibody; an anti-CD40 antibody; an anti-CD38 antibody; an anti-B7-H3 antibody; or an anti-CD47 antibody

### EXAMPLES

The following examples further illustrate aspects of the present disclosure. However, they are in no way a limitation of the teachings or disclosure of the present disclosure as set forth herein.

### Example 1: Development of the ADA1 Mutants

Here, the inventors have shown that administration of a human adenosine deaminase I enzyme (HsADA1) can slow tumor growth by degrading adenosine, and crucially, engineered variants of HsADA1 that have enhanced stability at biological conditions. Disclosed herein is a method to produce and purify the HsADA1 and associated variants at high yield. This example constitutes a new type of ‘metabolic cancer immunotherapy’ which is of great interest and great potential commercial value in the field of cancer therapeutics.

The inventors have shown that it is possible to utilize an adenosine degrading enzyme to target ADO in tumors, and that this enzyme-mediated depletion of ADO slows tumor growth in mice. More specifically, the inventors have shown that a highly active, pharmacologically optimized human adenosine deaminase 1 enzyme (HsADA1) can be administered to tumors to reduce tumor burden and prolong survival in a mouse model of colon cancer. This example method of enzyme-mediated depletion is a broadly applicable therapeutic route to stimulate antitumor immunity. Crucially, the inventors have further engineered a variant of HsADA1 with stabilizing mutations that allows it to better retain activity at biological conditions (37° C. and pH 7.4). This improves the enzyme's overall ability to degrade ADO in tumors to stimulate antitumor immunity. This use of the HsADA1 enzyme (and notably, an engineered and stabilized mutated variant of HsADA1, called HsADA1 K164E) to disrupt ADO-dependent immune suppression is an innovative strategy that has clear advantages over current strategies towards preventing ADO accumulation and suppressive signaling. For instance, pharmacological inhibitors of ADO synthesis do not completely block ADO production because other enzymes catalyze redundant reactions. If pan-inhibition of ADO synthesis were realized, ATP accumulation and lost purine nucleotide recycling ability could result in toxicities. Finally, as with any small molecule drug, the use of inhibitors would be expected to give rise to resistance through the expression of multi-drug resistance proteins. This example strategy to administer a therapeutic enzyme that eliminates ADO to disrupt immune suppression is not limited by any of these shortcomings, and the engineered K164E variant has never been generated before. The inventors have also created several other HsADA1 single mutation variants that have improved stability compared to wildtype HsADA1.

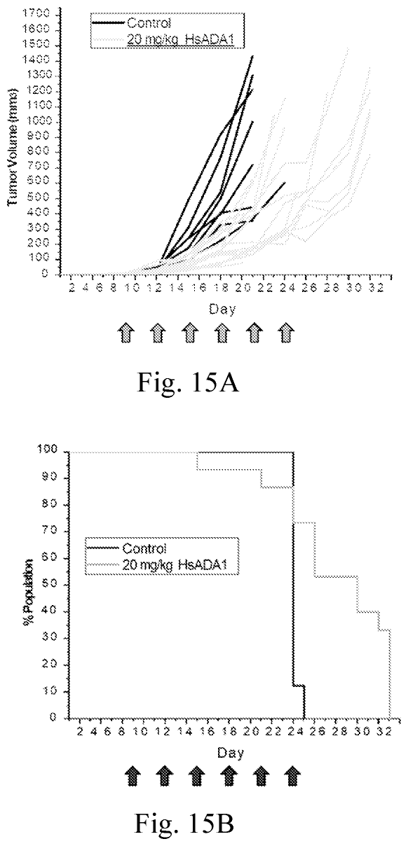

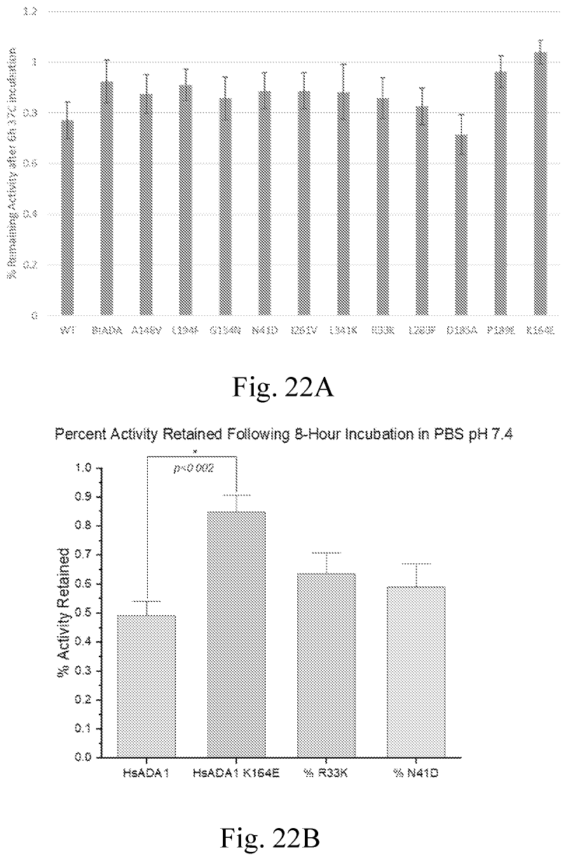

This is an example process to recombinantly produce the HsADA1 enzyme at high titer and high purity for therapeutic use. In addition, HsADA1 administration to tumors has never before been shown to slow tumor growth (see FIG. 15A). Finally, the inventors have engineered the amino acid sequence of the HsADA1 to create a variant denoted K164E. This HsADA1 variant has an aspartate residue and amino acid position 163 in place of a lysine. This variant has greatly improved stability at 37° C. due to its reduced tendency to aggregate. Also disclosed herein are several other HsADA1 variants that have slightly improved stability due to mutations in their amino acid code (see FIGS. 22A and 22B).

Recently it has been shown that an engineered variant of HsADA2 with 10-fold improved activity is able to stimulate anti-cancer immune responses to slow tumor growth in murine models of cancer. The inventors have shown that the more active HsADA1 enzyme can also slow tumor growth in a murine model of colon cancer (see FIG. 15A). Therefore, adenosine degrading enzymes have the general utility of slowing cancer growth, but the results seen with the present HsADA1 enzyme are unexpected (see FIG. 22A). The HsADA1 enzyme can outperform the HsADA2 enzyme because it has significantly higher activity, and importantly, the present engineered variants of HsADA1 that have improved stability should have an even better impact. The ability to perform these experiments was driven in part by developing a robust recombinant purification protocol for the HsADA1 enzyme by expressing it in E. coli. HsADA1 had never been purified to homogeneity recombinantly at high yield prior to recent work performed by the inventors.

The entire protein sequence for Human Adenosine Deaminase 1 (Uniprot P00813) was codon-optimized for Escherichia coli expression and synthesized as a linear DNA fragment by Twist Biosciences. Primers to attach a two hexahistidine tags separated by two glycine residues were purchased as custom oligonucleotides from Eurofins Genomics. Primers from Eurofins Genomics were also purchased to amplify the HsADA1-6His-GG-6His gene fragment while adding overlap to the pET28A plasmid, and a pET28A-HsADA1-6His-GG-6His plasmid was assembled through Gibson cloning. The product of the Gibson assembly was transformed into E. coli DH10B. Similarly, a pET28A-HsADA1-6His plasmid was constructed. Similarly, an alternative codon-optimization for E. coli that used a different nucleotide sequence was used to construct a plasmid dubbed pET28A-HsADA1-codonopt2-6His.

Alternatively or additionally, two codon-optimized sequences were originally generated and purchased, denoted as HsADA1-codonoptl and HsADA1-codonopt2. Originally, primers to attach a C′-terminus hexahistidine tag flanked by diglycine sequences on both ends (GG-6His-GG) were purchased as custom oligonucleotides (Eurofins Genomics). Additional custom oligonucleotides were purchased to amplify the entire HsADA1-GG-6His-GG gene fragment while adding overlap for the pET-28(a) plasmid between the Ncol and Sall restriction sites within the multiple cloning site. The Gibson assembly process was implemented to insert both either codon optimized HsADA1 sequence followed by a hexahistidine tag between the Ncol and Sall sites of the pET-28(a) plasmid. The Gibson assembly products were transformed into E. coli DH10B (New England Biolabs) via electroporation and selected for with luria broth agar plate doped with 50 μg/mL kanamycin sulfate. Sequences were confirmed following plasmid isolation through the Miniprep method (Qiagen) and Sanger sequencing performed by Eurofins Genomics or Genewiz.

Later, additional primers to append a decahistidine tag to the HsADA1-GG-6His-GG codon-optimized sequences were purchased from Eurofins Genomics to generate a final gene fragment with the sequence: HsADA1-GG-6His-GG-10His-GG. Additional primers were purchased to provide overlap between the Ncol and Sall restriction sites of the pET-28(a) plasmid (Eurofins Genomics) and plasmids were assembled and sequence-verified via the process described above. Later mutations to the HsADA1-codonoptl gene sequence were introduced via polymerase chain reaction and overlapping primers purchased from Eurofins Genomics. The mutated HsADA1-GG-6His-GG-10His-GG sequences were inserted between the Ncol and Sall restriction sites of the pET-28(a) plasmid as described.

An additional plasmid was constructed to allow for cleavage of the histidine tag(s) from the expressed HsADA1 protein. A tobacco etch virus (TEV) cleavage sequence was introduced at the C-terminus of the HsADA1-codonoptl gene sequence followed by a GG-6His-GG histidine tag using oligonucleotide primers (Eurofins Genomics). Additional oligonucleotide primers were purchased to amplify the HsADA1-TEV-GG-6His-GG sequence while adding overlap for the pET-28(a) plasmid between the Ncol and Xhol restriction sites within the multiple closing regions, which appended an additional 6His sequence to the transcribed gene sequence resulting in a HsADA1-TEV-GG-6His-GG-6His protein construct. The pET-28(a) HsADA1-TEV-GG-6His-GG-6His plasmid was transformed and verified as previously described.

For expression and harvest of His-tagged HsADA1 Proteins: single colonies of E. coli T7 Express (New England BioLabs), harboring the pET28A-HsADA1-6His-GG-6His plasmid, the pET28A-HsADA1-6His plasmid, or the pET28A-HsADA1-codonopt2-6His plasmid, were used to inoculate 25 mL LB doped with 50 μg/mL kanamycin sulfate, and grown overnight with shaking. 10 mL of overnight culture was used to inoculate 1 L of Terrific Broth, doped with 50 μg/mL kanamycin sulfate, in a 4 L flask. At an OD600-1.0, at which point the flasks were placed in an ice-water slurry and moved to a 4° C. cold room for 30 minutes. Following chilling, to each flask was added 3% v/v ethanol, and each culture was induced to a final concentration of 0.5 mM IPTG. The flasks were then kept for 48-56 hours at 15° C., shaking at 200 RPM, to induce His-tagged HsADA1 protein expression. Induced cultures were harvested by centrifugation at 3,400×g and 4° C. for 30 minutes, resuspended in phosphate-buffered saline pH 7.4 (PBS pH 7.4), condensed again by centrifugation (3,400×g and 4° C. for 30 mins), and stored at −80° C. for future use.

For purification of His-tagged HsADA1 proteins: Lysis buffer was prepared as 20 mM sodium phosphate pH 7.4, 300 mM sodium chloride, 20 mM imidazole (all purchased from Sigma Aldrich), 1 mM phenylmethylsulfonyl fluoride (Thermo Scientific), 25 U/mL Universal Nuclease (Pierce), and lmg/mL Lysozyme (Thermo Scientific). Frozen pellets were resuspended at a ratio of 10 mL chilled lysis buffer to lg pellet. Pellets were gently spun with a magnetic stir bar at 4° C. until resuspended. Resuspended cells were sonicated using a QSonica 500 Sonicator with a ‘A” tip attachment at 40% amplitude for a total of 30 sonication minutes adhering to cycles of 10 seconds on and 1 second off while chilled with an ice/water slurry. Lysate was aliquoted into 50 mL conical tubes (Eppendorf) and centrifugated for 1-1.5 hours at 20,000×g and 4° C. Clarified lysate was passed through sterile 0.22 μm GD/X Whatman filters (GE Healthcare) and loaded into a 150 mL Superloop (Cytiva). A 5 mL HisTrap High Performance (Cytiva) nickel column was used to purify the HsADA1 proteins with the aid of an AKTA pure (Cytiva) at 4° C. Buffer A was composed of 20 mM sodium phosphate pH 7.4, 300 mM NaCl, and 20 mM imidazole while Buffer B was composed of 20 mM sodium phosphate pH 7.4, 300 mM NaCl, and 500 mM imidazole. The purification sequence began as a 5-column volume (CV) equilibration of the column with Buffer A, followed by sample application, a 10CV wash with Buffer A, followed by a linear gradient from 0% to 100% Buffer B over 15CV all at a 1 mL/min flow rate. Fractions were collected in 1 mL aliquots using the Fraction Collector F9-C(Cytiva). Fractions were analyzed by SDS-PAGE, and pure fractions were pooled, sterile filtered using a 0.22 μm filter (Fisher), and concentrated using Amicon Ultra 15 Centrifugal Filter Unit Ultracel-10 (Millipore). Concentrated HsADA1 proteins was buffer exchanged into chilled PBS pH 7.4 using HiTrap Desalting Columns (Cytiva). Protein A280 was determined using the NanoDrop One/OneC UV-Vis Spectrophotometer (Thermo Scientific). Expasy was used to calculate molecular mass and extinction coefficients for His-tagged HsADA1. Notably, the using the pET28A-HsADA1-6His-GG-10His-GG plasmid for protein expression resulted in 10-15 mg/L of HsADA1 production, whereas the pET28A-HsADA1-6His plasmid and the pET28A-HsADA1-codonopt2-6His plasmids afforded far less HsADA1 (−lmg/L or less). In addition, the long induction time (>48 hours), low induction temperature (15° C.), and addition of 3% v/v ethanol were all necessary to afford high yield HsADA1 production, up to 40 mg/L with the K164E HsADA1 variant.

For purification of HsADA1 without His-Tags: in addition, an example protocol to generate HsADA1 that had had its His-Tag removed using a TEV-protease based system is disclosed herein. First, the inventors appended a TEV-protease cleavage site sequence and a GG-6His-GG-6His amino acid sequence to the HsADA1 gene and inserted it into pET28A to create a pET28A-HsADA1-TEV-GG-6His-GG-6His plasmid. Using this plasmid and the recombinant expression and purification methods described above, the inventors purified a HsADA1-TEV-6His-GG-6His protein. Then, HsADA1-TEV-6His-GG-6His was mixed with 6His-TEV(S219V)-Arg5 at a ratio of 10 mg to 1 mg. 6His-TEV(S219V)-Arg5 was produced using BL21(DE3) CodonPlus-RIL containing the pRK793 plasmid. The mixture was gently turned end-over-end at 4° C. for 16 hours and sterile filtered with GD/X Whatman 0.22 um filters to remove aggregation. Then, the mixture was diluted 10-fold with Buffer A and applied to the 150 mL Superloop. Once again, a 5 mL HisTrap HP column and AKTA Pure were used to obtain pure, cleaved HsADA1. 5CV of Buffer A was used to equilibrate the column, followed by sample application of which the column flow through was collected in a 50 mL conical tube. All purification steps were performed in a 4° C. refrigeration unit and the flow through the system was 1 mL/minute. The column flow through was subject to a 0.22 um filter, concentrated using a 10 kDa device, and buffer exchanged into PBS pH 7.4 using the HiTrap Desalting column as previously described. Purity of the cleaved HsADA1 without His-tags was assessed by SDS-PAGE and the concentration by A280 measurement.

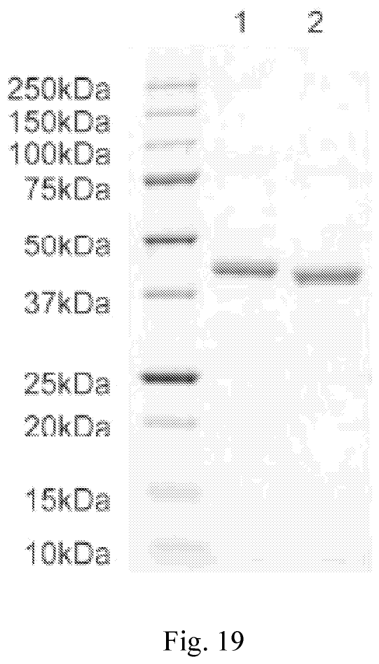

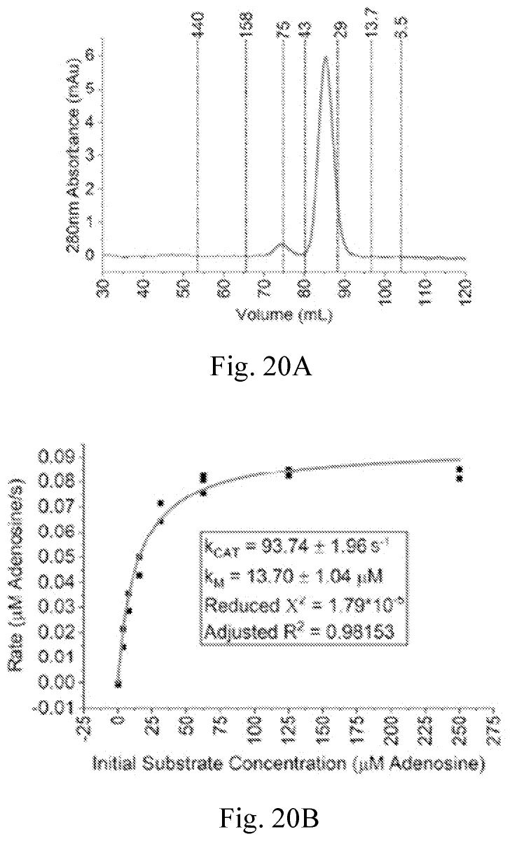

Relating to description and characterization of the human adenosine deaminase I protein: HsADA1 is a 41 kDa enzyme with a Zn2+ cofactor 22,23. The protein sequence of HsADA1 shares 89% identity with bovine adenosine deaminase (BtADA) and 83% with marine ADA (MmADA) homologs, and catalytic residues are conserved amongst the enzymes. SDS-PAGE analysis shows HsADA1 is >99% pure after nickel affinity chromatography, with an expected decrease in MW after TEV-cleavage of the 6His-GG-6his tags from 43.5 kDa to 41.6 kDa (see FIG. 19). Fractionation of pure cleaved HsADA1 by size exclusion chromatography confirms HsADA1 is predominantly monomeric in solution, with only a small dimeric HsADA1 population (see FIG. 20A).

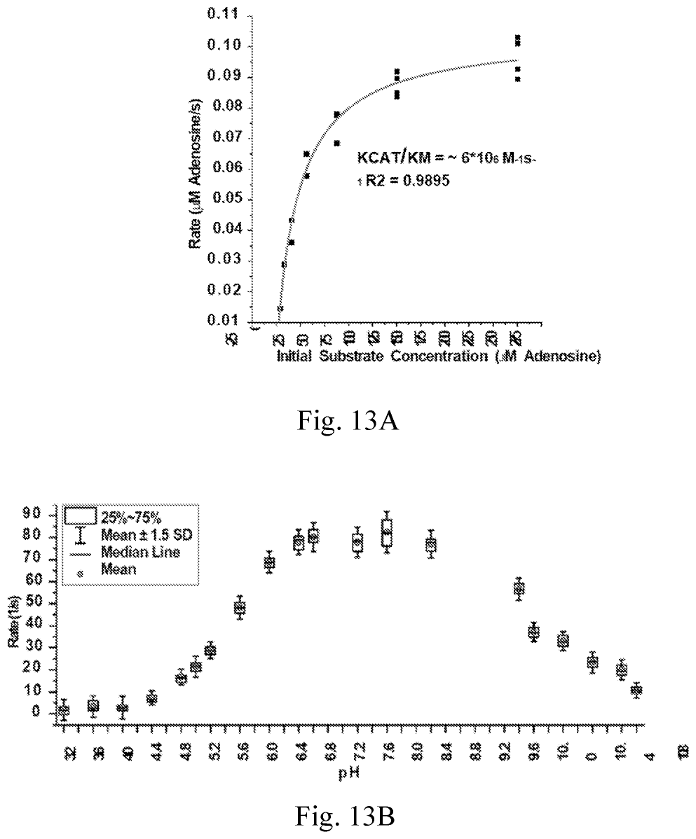

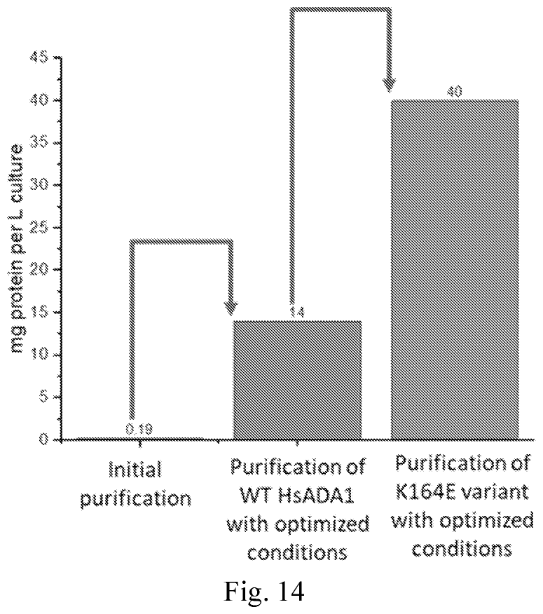

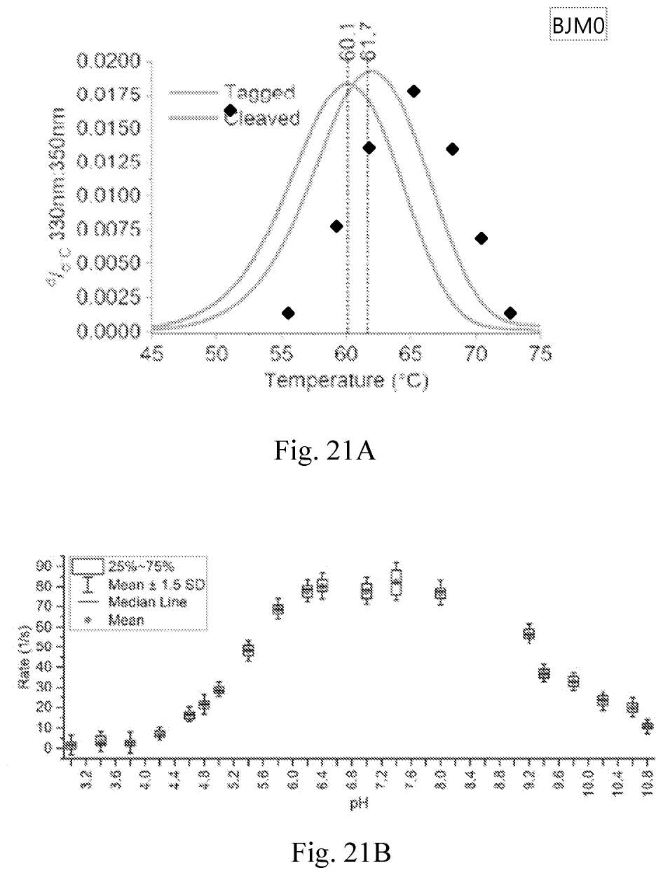

The inventors evaluated HsADA1 kinetics with its preferred substrate, adenosine (see FIG. 20B). Michaelis-Menten parameters for HsADA1 were determined via a 96-well assay method using substrate concentrations ranging from 0 to 250 uM adenosine and 0.001 uM to 0.005 uM HsADA1. In a 96-well UV-Transparent Microplate (Corning or Greiner Bio-One), 160 uL of 1.25× substrate (in PBS pH 7.4) solution was added to a 40 uL well containing 5× enzyme (in PBS pH 7.4) solution. Absorbance at 265 nm, as a readout of adenosine level, was monitored using a BioTek Synergy HT 96-well plate spectrometer. Adenosine degradation rate was calculated from the linear portion of the raw reaction curves corresponding with less than 10-percent substrate degradation and fitted to the Michaelis-Menten using OriginPro 2021. Non-linear regression analysis revealed catalytic parameters (kcat=93.74±1.96 s-1 and kM=13.70±1.04 μM, approximately kcat/kM=7.15×106 M-is-1), similar to the reported activities of the BtADA (kcat=385 s-1 and kM=43 μM, kcat/kM=8.95×106 M-is-1), and MmADA (kcat=240 s-1 and kM=21 μM, kcat/kM=1.14×107 M-ls-1) and nearly two orders of magnitude higher than for HsADA2 (kcat=88 s-1 and kM=2 mM) (see FIG. 20B). Prior characterization of recombinant HsADA1 catalytic activity (kcat=190 s-1 and kM=26 μM, kcat/kM=7.31×106 M-ls-1) is in agreement with the inventors' work. The activity of tagged (kcat=94.55±3.485−1 and kM=12.41±1.60 μM kcat/kM=7.62×106 M-ls-1) and cleaved HsADA1 are nearly identical, and its thermal stability is similarly unaffected (see FIG. 20A, FIG. 20B). Thus, the GG-6His-GG-6His tag and its removal does not interfere with catalysis or stability. Reactions conducted in a pH range between 3.0 and 10.8 demonstrate broad activity, with optimal pH values for HsADA1 degradation of adenosine at physiological pH range between 6.0 and 8.0 (see FIG. 21B, FIG. 13B).

HsADA1-GG-6His-GG-10His-GG was purified as described above and desalted and buffer exchanged into a HEPES-based buffer (20 mM HEPES, 150 mM NaCl, 0.1 mM CaC12 pH 7.5) using a HiTrap Desalting column (Cytiva). The HsADA1 protein was then purified of bacterial endotoxin using EndoTrap HDTM columns (Lionex GmbH), and then PEGylated by direct addition of 50-fold molar excess of Methoxy-PEG-CO(CH2)2COO—NHS, MW 5,000 Da (NOF America Corporation) followed by incubation at room temperature for 1 hour. HsADA1 was concentrated and buffer exchanged into PBS pH 7.4 using Amicon Ultra 15 Centrifugal Filter Unit Ultracel-30 (Millipore). Prior to storage, concentrated PEG-HsADA1 protein was diluted 1:1 with 30% v/v UltraPure Glycerol (ThermoFisher) in PBS pH 7.4 for a final concentration of approximately 1 mg/mL. Aliquots of PEG-HsADA1 were flash frozen in liquid nitrogen and stored at −80° C. for future use. Relating to the efficacy in syngeneic tumor mouse model towards slowing cancer growth: tumors were established in the hind flank of BALB/c mice by injecting 5×104 CT26 colon carcinoma cells. After tumors were palpable, either His-Tagged, endotoxin-cleaned, PEGylated HsADA1 protein or vehicle control was injected intratumorally at a dosage of 20 mg/kg for 6 total doses, which each does spaced three days apart. Mice treated with HsADA1 protein had longer overall survival due to reduced rate of CT26 tumor growth (see FIG. 15A).

Relating to variants of HsADA1 with improved stability at 37° C.: using molecular cloning techniques, the inventors introduced eleven single site mutations into HsADA1, generating 11 different HsADA1 variants, each with one amino acid mutation. These mutations include the following: R33K, N41D, G134N, A148V, K164E, D185A, P189E, L194F, 1261V, L283F, and 341K. In an E. coli cell lysate assay at 37° C., ten of the variants (all but D185A) showed enhanced retention of activity compared to wildtype HsADA1. The inventors further purified the K164E, R33K, and N41D variants to homogeneity and performed a test in PBS at 37° C., showing that all three variants retained activity better than wildtype HsADA1 as purified enzymes. In particular, the HsADA1 K164E variant had more than 80% better retention of activity after 8 hours at 37° C. compared to wildtype HsADA1. Therefore, this K164E variant is far better suited for use as an enzyme immunotherapy than wildtype HsADA1, along with the other 9 improved variants, and all combinations of mutations that have further improved stability at 37° C. (see FIG. 22A and FIG. 22B).

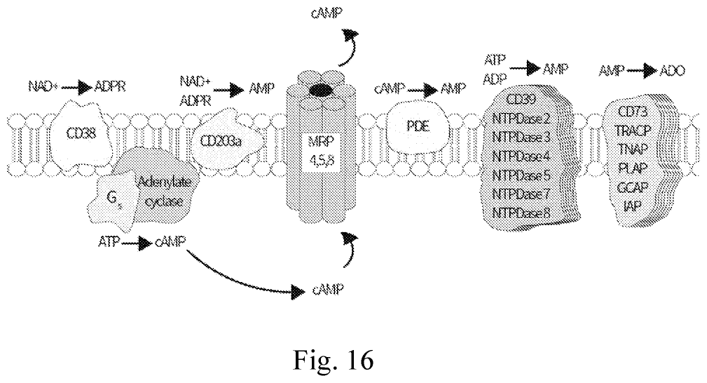

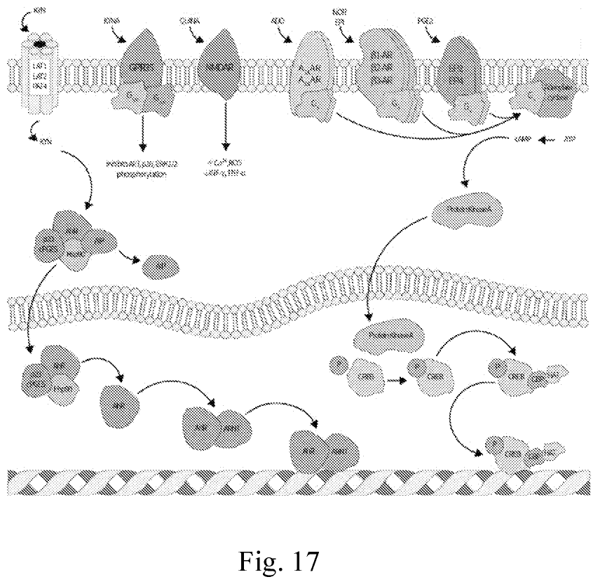

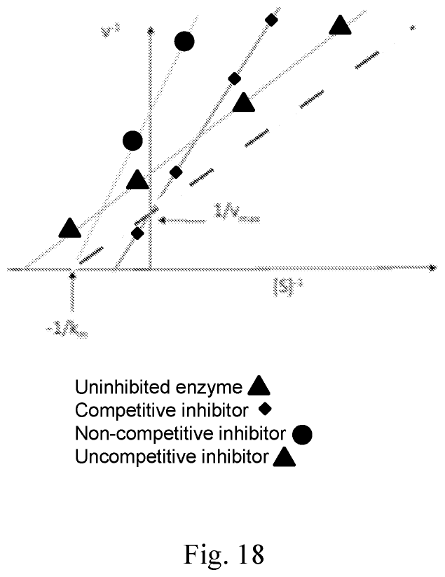

A key advantage of this enzyme therapeutic technology is that it targets adenosine directly. Therefore, it is not limited by redundancies in adenosine synthesis and signaling, i.e., that multiple metabolic pathways, each of which can employ multiple enzyme homologs, can produce adenosine (see FIG. 16), and that adenosine suppresses immune cells responses by signaling through multiple receptors found on immune cells. Other therapies that target only one synthesis pathway or attempt to prevent adenosine from impacting only one receptor would be limited by these redundancies. In addition, the present HsADA1 variants are much more active than the wildtype HsADA1, and HsADA1 is not immunogenic, like BtADA1 (Bos taurus adenosine deaminase 1). Finally, these example HsADA1 variants have improved stability at biological stability, allowing for their therapeutic use, whereas the wildtype HsADA1 enzyme would aggregate.

In more detail, the most studied pathway of adenosine synthesis has two steps: hydrolysis of ATP to AMP via an ectonucleoside triphosphate diphosphohydrolase (ENTPDase), and then hydrolysis of AMP to adenosine by a 5′-nucleotidase (5′NTDase). The hypoxic tumor microenvironment promotes ATP release into the extracellular space by stressed, dead and dying cells, ensuring substrate availability. The membrane-anchored enzymes CD39 (ENTPDase) and CD73 (5′NTDase) have been broadly implicated in cancer and can be upregulated on cancer and immune cells, though numerous other ecto-enzymes catalyze hydrolysis of ATP or AMP. Adenosine can also be synthesized from NAD+ in two related extracellular metabolic pathways. In one, CD203a degrades NAD+ into nicotinamide mononucleotide and AMP, and in the other, CD38 converts NAD+ into nicotinamide and adenosine diphosphate ribose, which is then cleaved by CD203a into pyrophosphate and AMP. AMP can then be dephosphorylated into adenosine by a 5′NTDase, an alkaline phosphatase (AP), or the tartrate-resistant acid phosphatase enzyme (TRACP). Connexin 43 allows for intracellular NAD+ to traverse the cell membrane. Similarly, adenosine can be synthesized from cAMP through two metabolic pathways. In one, ten adenylate cyclase isoforms catalyze the conversion of extracellular ATP to cAMP, and in the other, intracellular cAMP is secreted via the multidrug resistance proteins 4, 5, and 8 (MRP4,5,8). cAMP is converted to AMP through an ecto-phosphodiesterase (likely to be an isoform from one of the phosphodiesterase (PDE) super families 4,7,8,10,11), and AMP is dephosphorylated into adenosine. Extracellular AMP may instead be deaminated by AMP deaminase to form inosine monophosphate (IMP), which can be dephosphorylated by CD73 to form inosine 23. Adenosine deamination, catalyzed by either adenosine deaminase 1 or 2, also yields inosine. Therefore, adenosine synthesis can occur through multiple distinct pathways, in which each enzymatic step can be catalyzed by multiple enzymes (see FIG. 16).

In addition, adenosine suppresses immune cells responses by signaling through multiple receptors found on immune cells, particularly A2AAR and A2BAR. In more detail, adenosine can agonize four G protein-coupled extracellular receptors, AJAR, A2AAR, A2BAR, and A3AR. In particular, agonism of the high affinity A2A or low affinity A2B adenosine receptors stimulates production of cyclic adenosine monophosphate (cAMP) from ATP, which acts through Protein Kinase A (PKA) and cAMP-response element binding protein (CREB) to inhibit inflammatory signaling pathways and suppress immune responses. Agonism of either A2AAR or A2BAR results in immunosuppression, and immune cells upregulate both receptors under hypoxic conditions. For instance, in CD8′ T cells, A2AAR agonism reduces cytotoxicity, inflammatory cytokine production, and TCR-mediated signaling. A2AAR agonism further inhibits inflammatory cytokine production by neutrophils, macrophages, and DCs, and promotes immunosuppressive macrophage behavior. MDSCs also express A2AAR in the tumor, and its agonism results in increased IL-10 production. IL-10 has been shown to behave in a dual role to lessen inflammatory response and drive tumor progression in a tumor- and context-specific manner. Adenosine agonizes the lower affinity A2BAR receptor with similar effects, diminishing CD4′ T cell inflammatory function, reducing NK cytotoxicity, inhibiting neutrophil superoxide production and oxidative burst, and downregulating expression of nitric oxide synthase while increasing IL-10 production by macrophages. Adenosine signaling through A2AAR or A2BAR further promotes differentiation of tolerogenic immune cells, while acting as a signaling molecule to help mediate their impact. Therefore, both ADO synthesis and signaling have redundancies.

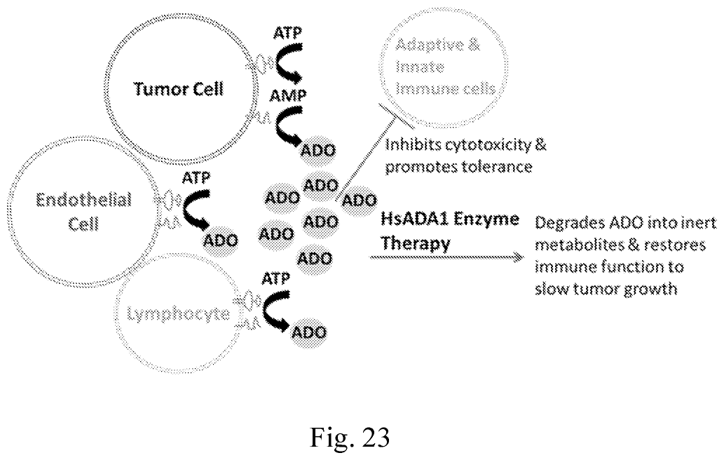

As mentioned above, there are three small molecule antagonists of A2AAR and four CD73-inhibiting antibodies in seventeen total phase I/II clinical evaluations, and a CD39 inhibitor and aCD39 antibody are in preclinical development as of September 2021. However, these efforts suffer from important shortcomings due to the enzymatic redundancies and receptor redundancies described above. The present example circumvents these limitations by using an enzyme, HsADA1 (and/or its engineered derivatives including but limited to the K163D variant), to directly degrade adenosine by administering the enzyme to a solid tumor (see FIG. 23).

This invention could be used directly as a cancer immunotherapy. The main commercial application for this technology (the ability to purify high titers of HsADA1, its use as an immunotherapy, and the use of optimized/engineered variants of HsADA1 that have enhanced stability) is as a cancer therapeutic. In this vein, recent work in the field, via a pan-cancer analysis of transcriptomic signatures of 9145 tumor samples, demonstrated that high ‘ADO signaling’ in tumors correlates with poor survival. Importantly, high ADO levels also predicted poor response to PD-1 blockade in 65 patients with NSCLC, HNSCC, or skin cutaneous melanoma. Therefore, using HsADA1 and importantly, enhanced HsADA1 variants, to eliminated ADO to prevent this ‘ADO’ signaling could become a very important therapeutic across cancer types.

Indirect use for ADA-SCID: In addition, this invention could be used directly as an enzyme replacement therapy for a rare genetic condition called ADA-SCID, in which a person is born without a functioning HsADA1 gene. ADA-SCID resulting from HsADA1 deficiency is curable with bone marrow transplants but is commonly treated with an enzyme replacement therapy via administration of a recombinant ADA1 from cows, i.e., Bos taurus adenosine deaminase 1 or BtADAl. BtADA1 is sold under the brand names Adagen and Revcovi, and its administration prevents deoxy-ADO cytotoxicity to lymphocytes, allowing the development of a somewhat weakened adaptive immune system. BtADA1 is a clinically approved therapy, but because it is of non-human origin, it elicits anti-drug antibodies that limit its effectiveness, even in ADA-SCID patients with weakened immune systems.

Finally, this example provides a production and purification method for HsADA1, using recombinant E. coli cells cultured and an optimized expression and purification protocol, to allow high titer, high yield production of HsADA1. This protocol can be used to produce the enzyme for the described therapeutic purposes.

### Example 2: Characterization of the HsADA1 Crystal Structure: Catalytically Active Holo Homo sapiens Adenosine Deaminase I Adopts a Closed Conformation

Contrary to expectations from orthologous structures in mouse and cow, the structure of holo human adenosine deaminase 1 (HsADA1) disclosed herein shows it adopts a closed conformation at the entry of its active site. This finding poses a cautionary tale for reliance on homologs for structural inference relevant to applications such as protein engineering or drug development.

Homo sapiens adenosine deaminase 1 (HsADA1, Uniprot P00813) is an immunologically relevant enzyme with roles in T cell activation and modulation of adenosine metabolism and signaling. Patients with HsADA1 genetic deficiency suffer from severe combined immunodeficiency, and HsADA1 is a therapeutic target in Hairy Cell Leukemias. Historically, insights into the catalytic mechanism and the structural attributes of HsADA1 have been derived from studies of its homologs from Bos taurus (BtADA) and Mus musculus (MmADA). Disclosed herein is the structure of holo HsADA1, as well as a biochemical characterization that confirms its high activity and shows that it is active across a broad pH range. Structurally, holo HsADA1 adopts a closed conformation, distinct from the open conformation of holo BtADA. Comparison of holo HsADA1 and MmADA reveals MmADA also adopts a closed conformation. These findings challenge previous assumptions gleaned from BtADA regarding the conformation of HsADA1 that may be relevant to its immunological interactions, particularly its ability to bind adenosine receptors. From a broader perspective, this structural analysis of HsADA1 presents a cautionary tale for reliance on homologs for structural inference relevant to applications such as protein engineering or drug development.

Adenosine deaminase enzymes irreversibly convert adenosine and 2-deoxyadenosine to inosine and 2-deoxyinosine, respectively, contributing to purine metabolism across prokaryotic and eukaryotic organisms. The human genome encodes two adenosine deaminases, Homo sapiens adenosine deaminase I, HsADA1, and adenosine deaminase II, HsADA2. The expression profiles, amino acid sequences, and binding partners of HsADA1 and HsADA2 are distinct, though they share catalytic mechanisms. HsADA1 is primarily an intracellular enzyme but can be found as an ectoenzyme in complex with the membrane proteins CD26, an activation and co-stimulatory molecule expressed on the surface of T, B, and NK immune cell subsets, and the adenosine receptor (AR) subtypes AJAR, A2AAR, and A2BAR. By contrast, HsADA2 is secreted into serum and can bind proteoglycans.

Early analyses of adenosine deaminase activity in human tissues indicating a maximum in the spleen and high activity in intestinal tissue, as well as high activity in thymocytes and circulating lymphocytes. These studies did not distinguish between deaminase activity from HsADA1 and HsADA2, but data from the Human Protein Atlas confirm enriched HsADA1 expression in the blood, intestine, and lymphoid tissue. Patients lacking a functional HsADA1 suffer from a Severe Combined Immunodeficiency (ADA-SCID) characterized by reduced B, NK, and T cell counts, caused by increased deoxyribonucleotide levels that prevent lymphocyte maturation and cause their death. In ADA-SCID patients, the subsequent defects in lymphocyte-mediated immunity result in eventual fatality if not treated with bone marrow transplant or enzyme replacement therapy. Long-term administration of a polyethylene glycol (PEG)-conjugated bovine adenosine deaminase (Pegademase and Elapegademase) benefits ADA-SCID patients by supporting immature lymphocyte development and reconstitution of functional immunity. Conversely, elevated HsADA1 levels have been associated with inflammatory disease and hematological malignancies, sparking interest in inhibitor development. In particular, the adenosine deaminase inhibitor pentostatin (T-deoxycoformycin) is used to treat Hairy Cell Leukemia and has been studied as a treatment of graft versus host disease.

BtADA has been shown to allosterically modulate the agonist affinity of adenosine receptors A2AAR and A2BAR in vitro to increase their sensitivity and heighten intracellular cAMP signaling relevant to immunosuppressed phenotypes. Further studies with BtADA have established the possibility of a molecular bridge between CD26-ADA-A2A/BAR connecting T cells with dendritic cells, however, the immunologic significance of the interaction is unknown. HsADA1 binding to A2AAR has also been confirmed in vitro, as well as HsADA1-mediated modulation of AJAR signaling.

HsADA1 is a 41 kDa enzyme with a Zn2′ cofactor. The protein sequence of HsADA1 shares 89% identity with bovine adenosine deaminase (BtADA) and 83% with murine ADA (MmADA) homologs, and catalytic residues are conserved amongst the enzymes. While HsADA1 and BtADA are able to complex with CD26, MmADA does not. Structural studies of BtADA and MmADA reveal a triosephosphate isomerase (TIM)-barrel topology that can adopt ‘open’ or ‘closed’ conformations, characterized by a subtle shift in a structural gate leading towards the substrate binding pocket. Based on these descriptions, holo (i.e., metalated but non-ligand bound) adenosine deaminase enzymes are thought to take the open conformation, as seen with holo BtADA, while substrate (adenosine, deoxyadenosine) and inhibitors that mimic substrate are thought to stabilize the closed conformation. Interestingly, holo MmADA appeared to take the closed conformation during an initial structural study, though this was attributed to binding of glycerol from the cryoprotectant.

While several crystallization studies of BtADA and MmADA have been described, a crystal structure of HsADA1 has yet to be reported in the literature. Disclosed herein is kinetic characterization of high-purity, high-yield recombinantly produced HsADA1 and describe the structure of holo HsADA1. Strikingly, although it has a similar overall structure to its homologs, holo HsADA1 takes on an unexpectedly closed conformation with a noticeable shift in its structural gate compared to holo BtADA. Previously, adenosine deaminase enzymes were expected to maintain an open conformation until ligand binding. The inventors' structural result has implications for future inhibitor development and for the immunological functions of HsADA1.

### Materials and Methods

For macromolecule production and characterization: the gene sequence for HsADA1 (Uniprot P00813) was codon-optimized for Escherichia coli expression and synthesized as a linear DNA fragment by Twist Biosciences. Unless otherwise noted, all oligonucleotide primer sequences were purchased from Eurofins Genomics and all restriction enzymes from New England Biolabs. To add the first C-terminal Gly-Gly-His-His-His-His-His-His-Gly-Gly (SEQ ID NO: 300) (GG-6His-GG), two polymerase chain reaction (PCR) steps were employed. First, using the HsADA1 gene as the template and oligonucleotide primer sequences (Forward: 5′-AACTTTAAGAAGGAGATATACCATGGCTCAAACTCCGGCCTTCGAC (SEQ ID NO: 287), Reverse: 5′-TGGTGGTGATGATGACCGCCCAAGTTCTGGCCCGCGCTTG (SEQ ID NO: 288)), the first portion of the GG-6HIS-GG sequence was appended to the C′ terminus of the HsADA1 gene. The second PCR step used the first PCR as a template and additional primers (Forward (5′-3′): 5′-AACTTTAAGAAGGAGATATACCATGGCTCAAACTCCGGCCTTCGAC (SEQ ID NO: 287), Reverse: 5′-TCGAGTGCGGCCGCAAGCTTGTCGACTTAGCCGCCGTGATGGTGGTGATGATGAC CGCC (SEQ ID NO: 301) to complete the addition of GG-6His-GG. This PCR product was subsequently inserted into the pET-28(a)+ backbone between the Ncol and Sall restriction sites through Gibson Assembly. Next, a second hexahistidine tag was appended to the HsADA1-GG-6His-GG sequence by amplifying the region (Forward: 5′-GGAATTGTGAGCGGATAACAATTCCCC (SEQ ID NO: 291), Reverse: 5′-CAGTGGTGGTGGTGGTGGTGGCCGCCGTGATGGTGGT (SEQ ID NO: 292)) with overhang for the C′ terminus hexahistidine tag flanking the Xhol site in the pET-28a(+) multiple closing site. This fragment was inserted between the NcoI and Xhol sites of pET-28(a)+ using Gibson Assembly for a final plasmid construction of pET-28(a)+ HsADA1-GG-6His-GG-6His. To insert a tobacco-etch virus (TEV) protease cut site between the HsADA1 and GG-6His-GG-6His sequences, respectively, the entirety of the plasmid was constructed and assembled from three fragments. First, the TEV site was appended to the C′ terminus of the HsADA1 gene using pET-28(a)+ HsADA1-GG-6His-GG-6His as a template and oligonucleotide primers (Forward: 5′-GGGGAATTGTGAGCGGATAACAATTCCCCTC (SEQ ID NO: 294), Reverse: 5′-GCCGCCGTGATGGTGGTGATGATGACCGCCGGATTGGAAGTACAGGTTCTCCAA GTTCTGGCCCGCGCTTG (SEQ ID NO: 295)) to generate the first PCR product. The second PCR fragment also made use of pET-28(a)+ HsADA1-GG-6His-GG-6His as a template, amplifying the entirety of the GG-6His-GG-6His region to the middle of the kanamycin resistance gene of pET-28(a)+ (Forward: 5′-GGCGGTCATCATCACCACCATCAC (SEQ ID NO: 296), Reverse: 5′-ACTGCGATCCCCGGGAAAAC (SEQ ID NO: 297)). The third PCR fragment amplified the second portion of the Kanamycin resistance gene and the remainder of the pET-28(a)+ backbone through the lac operon to the multiple cloning site using pET-28(a)+ as the PCR template (Forward: 5′-CTTCTAATACCTGGAATGCT (SEQ ID NO: 298), Reverse: 5′-GGTATATCTCCTTCTTAAAGTTAAA (SEQ ID NO: 299)). Next, the three PCR fragments were combined through Gibson Assembly to form the pET-28(a)+ HsADA1-TEV-GG-6His-GG-6His plasmid. See table 1.

The product of the final Gibson Assembly was transformed into E. coli DH10B (New England Biolabs). Transformants were plated onto Luria-Bertani (LB) agar plates (LB powder from Fisher Bioreagents, and agar from Teknova) containing 50 μg/mL kanamycin sulfate (Sigma Aldrich). Single colonies were used to inoculate cultures with 5 mL of LB and 50 μg/mL kanamycin sulfate that were grown overnight prior to plasmid extraction using the QIAprep Spin Miniprep Kit (Qiagen) and sequence confirmation through Sanger Sequencing (Eurofins Genomics).

For expression and harvest of HsADA1-TEV-6His-GG-6His: a single colony of E. coli T7 Express (New England BioLabs), harboring the pET28A-HsADA1-TEV-6His-GG-6His plasmid, was used to inoculate 25 mL LB supplemented with 50 μg/mL kanamycin sulfate, and grown overnight with shaking. 10 mL of overnight culture was used to inoculate 1 L of Terrific Broth (Invitrogen), doped with 50 μg/mL kanamycin sulfate, in a 4 L flask. At an OD600-1.0, the flasks were chilled briefly, and then induced with 0.5 mM IPTG (Fisher Bioreagents) for at least 48 hours with shaking. Induced cultures were harvested by centrifugation at 3,400×g and 4° C. for 30 minutes and stored at −80° C.

For purification of HsADA1-TEV-6His-GG-6His protein: lysis buffer was prepared as 20 mM sodium phosphate pH 7.4, 300 mM sodium chloride, 20 mM imidazole (all purchased from Sigma Aldrich), 1 mM phenylmethylsulfonyl fluoride (Thermo Scientific), 25 U/mL Universal Nuclease (Pierce), and lmg/mL Lysozyme (Thermo Scientific). Frozen pellets were resuspended at a ratio of 5 mL chilled lysis buffer to 1 g pellet. Pellets were gently spun with a magnetic stir bar at 4° C. until resuspended. Resuspended cells were sonicated using a QSonica 500 Sonicator with a ½″ tip attachment at 40% amplitude for a total of 30 sonication minutes adhering to cycles of 5 seconds on and 1 second off. Lysate was aliquoted into 50 mL conical tubes (Eppendorf) and centrifugated for an hour at 20,000×g and 4° C. Clarified lysate was passed through sterile 0.22 μm GD/X Whatman filters (GE Healthcare) and loaded into a 150 mL Superloop (Cytiva). A 5 mL HisTrap High Performance (Cytiva) nickel affinity column on an AKTA Pure (Cytiva) system at 4° C. was used to purify HsADA1-TEV-6His-GG-6His. After 5 column volume (CV) equilibration of with Buffer A (20 mM sodium phosphate pH 7.4, 300 mM NaCl, and 20 mM imidazole), the sample was applied, washed with 10CV Buffer A, followed by elution with a linear gradient from 0% to 100% Buffer B (20 mM sodium phosphate pH 7.4, 300 mM NaCl, and 500 mM imidazole) over 15CV. Fractions were analyzed by SDS-PAGE, and pure fractions were pooled, sterile filtered using a 0.22 μm filter (Fisher), and concentrated using Amicon Ultra 15 Centrifugal Filter Unit Ultracel-10 (Millipore). Concentrated HsADA1-TEV-6His-GG-6His was buffer exchanged into chilled PBS pH 7.4 using HiTrap Desalting Columns (Cytiva). Protein A280 was determined using the NanoDrop One/OneC UV-Vis Spectrophotometer (Thermo Scientific). Expasy was used to calculate molecular mass and extinction coefficients for full length and cleaved HsADA1.

For cleavage of HsADA1-TEV-6His-GG-6His protein: HsADA1-TEV-6His-GG-6His was mixed with 6His-TEV(S219V)-Arg5 at a ratio of 10 mg to 1 mg. 6His-TEV(S219V)-Arg5 was produced using BL21(DE3) CodonPlus-RIL containing the pRK793 plasmid. The mixture was gently turned end-over-end at 4OC for 16 hours and sterile filtered with GD/X Whatman 0.22 μm filters. Then, the mixture was diluted 10-fold with Buffer A and applied to the 150 mL Superloop. The protocol described above used to obtain near homogeneity, untagged HsADA1, and the column flow through was passed through a 0.22 μm fil, concentrated using a 10 kDa device, and buffer exchanged into PBS pH 7.4 using the HiTrap Desalting column as previously described. Purity of the untagged HsADA1 was assessed by SDS-PAGE and the concentration by A280 nm measurement.

TABLE 1
Macromolecule production information

| Source | |
|---|---|
| organism | Homo sapiens |
| DNA | Twist Biosciences (gene), |
| source | Eurofins (primers) |
| Plasmid 1 | |
| PCR1 | |
| Template | Synthesized HsADA1 gene |
| Forward | 5′-ACTTTAAGAAGGAGATATACCATGGCTCA |
| primer | AACTCCGGCCTTCGAC (SEQ ID NO: 287) |
| Reverse | 5′-TGGTGGTGATGATGACCGCCCAAGTTCTG |
| primer | GCCCGCGCTTG (SEQ ID NO: 288) |
| Expression | pET-28(a)+ |
| vector | |
| PCR2 | |
| Template | Plasmid 1 PCR I |
| Forward | 5′-ACTTTAAGAAGGAGATATACCATGGCTCA |
| primer | AACTCCGGCCTTCGAC (SEQ ID NO: 287) |
| Reverse | 5′-CGAGTGCGGCCGCAAGCTTGTCGACTTAG |
| primer | CCGCCGTGATGGTGGTGATGATGAC-CGCC |
| | (SEQ ID NO: 289) |
| Gibson | Fragment 1: Plasmid 1 PCR I |
| Assembly | Fragment 2: pET-28a(+) |
| | (digested with Ncol and Sall) |
| Plasmid ID | pET-28a(+) HsADA1-GG-6His-GG |
| Complete | MAQTPAFDKPKVELHVHLDGSIKPETILYYGR |
| amino acid | RRGIALPANTAEGLLNVIGMDKPLTLPDFLAK |
| sequence of | FDYYMPAIAGCREAIKRIAYEFVEMKAKEGVV |
| the | YVEVRYSPHLLANSKVEPIPWNQAEGDLTPDE |
| construct | VVALVGQGLQEGERDFGVKARSILCCMRHQPN |
| produced | WSPKVVELCKKYQQQTVVAIDLAGDETIPGSS |
| | LLPGHVQAYQEAVKSGIHRTVHAGEVGSAEVV |
| | KEAVDILKTERLGHGYHTLEDQALYNRLRQEN |
| | MHFEICPWSSYLTGAWKPDTEHAVIRLKNDQA |
| | NYSLNTDDPLIFKSTLDTDYQMTKRDMGFTEE |
| | EFKRLNINAAKSSFLPEDEKRELLDLLYKAYG |
| | MPPSASAGQNLGGHHHHHHGG |
| | (SEQ ID NO: 290) |
| Plasmid 2 | |
| PCR1 | |
| Template | pET-28(a) HsADA1-GG-6His-GG |
| Forward | 5′-GGAATTGTGAGCGGATAACAATTCCCC |
| primer | (SEQ ID NO: 291) |
| Reverse | 5′-CAGTGGTGGTGGTGGTGGTGGCCGCCGTG |
| primer | ATGGTGGT (SEQ ID NO: 292) |
| Gibson | Fragment 1: Plasmid 2 PCR I |
| Assembly | Fragment 2: pET-28a(+) |
| | (digested with Ncol and XhoI) |
| Plasmid ID | pET-28a(+) HsADA1-GG-6His- |
| | GG-6His |
| Complete | HsADA1-GG-6His-GG-6His |
| amino acid | |
| sequence of | |
| the | |
| construct | |
| produced | |
| Plasmid 3 | |
| PCR1 | |
| Template | pET-28a(+) HsADA1-GG-6His- |
| | GG-6His |
| Forward | 5′-GGGGAATTGTGAGCGGATAACAATTCCC |
| primer | CTC (SEQ ID NO: 294) |
| Reverse | 5′-GCCGCCGTGATGGTGGTGATGATGACCG |
| primer | CCGGATTGGAAGTACAGGTTCTCCAAGTTCT |
| | GGCCCGCGCTTG (SEQ ID NO: 295) |
| PCR2 | |
| Template | pET-28a(+) HsADA1-GG-6His- |
| | GG-6His |
| Forward | 5′-GGCGGTCATCATCACCACCATCAC |
| primer | (SEQ ID NO: 296) |
| Reverse | 5′-ACTGCGATCCCCGGGAAAAC |
| primer | (SEQ ID NO: 297) |
| PCR3 | |
| Template | pET-28a(+) |
| Forward | 5′-CTTCTAATACCTGGAATGCT |
| primer | (SEQ ID NO: 298) |
| Reverse | 5′-GGTATATCTCCTTCTTAAAGTTAAA |
| primer | (SEQ ID NO: 299) |
| Gibson | Fragment 1: Plasmid 3 PCR I |
| Assembly | Fragment 2: Plasmid 3 PCR II |
| | Fragment 3: Plasmid 3 PCR III |
| Plasmid ID | pET-28a(+) HsADA1-TEV-GG-6His- |
| | GG-6His |

For kinetic analysis of HsADA1: kinetic parameters for HsADA1 were determined via a 96-well assay method using substrate concentrations ranging from 0 to 250 μM adenosine and 0.001 μM to 0.005 μM HsADA1. In a 96-well UV-Transparent Microplate (Corning), 160 uL of 1.25× substrate solution was added to a 40 μL well containing 5× enzyme solution. Absorbance at 265 nm, as a readout of adenosine level, was monitored using a BioTek Synergy HT 96-well plate spectrometer. Adenosine degradation rate was calculated from the linear portion of the raw reaction curves corresponding with less than 10-percent substrate degradation. Nonlinear regression analysis was performed with OriginPro 2021 software. The pH of each buffer solution was established with 50 mM of buffer salt ratio corresponding to the following pH ranges: 1) pH 3.0-5.4; citric acid:sodium citrate, 2) pH 5.8-8.0; sodium phosphate monobasic:sodium phosphate dibasic, and 3) pH 9.2-10.8; sodium carbonate:sodium bicarbonate.

For differential scanning fluorimetry: differential scanning fluorimetry was performed using a NanoTemper Prometheus NT.48 NanoDSF. Protein was loaded into Prometheus NT.48 glass capillaries at a concentration of lmg/mL in 50 mM sodium phosphate pH 7.4. NanoTemper PR.ThermoControl v2.1.5 software was used to visualize the absorbance curves at 330 nm and 350 nm, the curve of the absorbance ratio 330 nm:350 nm, and the first derivative of the absorbance ratio curve as temperature was ramped from 20° C. to 90° C. at a rate of 0.5° C. per minute.

Relating to crystallization (see table 2): purified, cleaved HsADA1 was exchanged to 10 mM HEPES pH 7.5, 150 mM NaCl and concentrated to 23 mg/mL using an Amicon filtration unit with a 3 kDa MW cut-off using 6 concentration and dilution steps. Crystals grew within −6 months at 20° C. in a sitting drop containing 1:1 (v/v) of 23 mg/mL HsADA1 from a mother liquor solution containing 0.49 M sodium phosphate monobasic monohydrate, 0.91 M potassium phosphate dibasic, pH 6.9. Crystals were cryoprotected by brief incubation in −2 M LiSO4 followed by flash cooling in liquid nitrogen.

TABLE 2
Crystallization

| | |
|---|---|
| Method | Vapor diffusion, sitting drop |
| Plate type | Intelli-Plate 96-3 LVR |
| Temperature (K) | 293 K |
| Protein concentration | 23 mg/ml |
| Buffer composition | 19 mM HEPES pH 7.5, 150 mM NaC1 |
| of protein solution | |
| Composition of | 0.49M sodium phosphate monobasic |
| reservoir solution | monohydrate, 0.91M potassium |
| | phosphate dibasic pH 6.9 |
| Volume and ratio of drop | 0.33 ul, 1:1 |
| Volume of reservoir | 50 ul |

For data collection and processing (see table 3): diffraction data were collected at the Southeast Regional Collaborative Access Team (SER-CAT) 22-ID beamline and processed using HKL-2000. The crystals were not singular, affecting the overall completeness of the dataset.

TABLE 3
Data collection and processing (Values for
the outer shell are given in parentheses)

| Diffraction source | SER-CAT 22-ID (APS) | |
|---|---|---|
| Wavelength (A) | 1.0 | |
| Temperature (K) | 100 K | |
| Detector | Dectris Eiger X16M | |
| Space group | P21 | |
| a, b, c (A) | 80.91, 49.52, 89.09 | |
| a, R, 7 (°) | 90, 96.18, 90 | |
| Resolution range (A) | 42.17-2.59 | (2.68-2.59) |
| Total No. of reflections | 42494 | (874) |
| No. of unique reflections | 17712 | (463) |
| Completeness (%) | 75.67 | (21.09) |
| Redundancy | 2.4 | (1.4) |
| (I/G(I)) | 7.39 | (1.75)t |
| Rmerge | 0.1201 | (0.3697) |

#Completeness issues derive from multiple lattice issues in the crystalline sample. t I/6(I) falls below 2.0 at 2.7 A resolution. Data were included to 2.6 A resolution because of the CC1/2 value in the highest resolution bin (0.75).

Relating to structure solution and refinement: the structure was solved by molecular replacement in Phaser using the polypeptide chain from PDB code 3IAR as a search model. The HsADA1 model was iteratively built and refined using Coot and Phenix. The structure was deposited to the PDB with accession code 7RTG. See table 4.

TABLE 4
Structure solution and refinement (Values for
the outer shell are given in parentheses)

| | | |
|---|---|---|
| Resolution range (A) | 42.17-2.59 | (2.68-2.59) |
| Completeness (%) | 75.67 | (21.09) |
| G cutoff | 7.39 | (1.75) |
| No. of reflections, working set | 16809 | (463) |
| No. of reflections, test set | 1679 | (46) |
| Final Reryst | 0.1713 | (0.3615) |
| Final Rfree | 0.2673 | (0.4520) |

| | |
|---|---|
| No. of non-H atoms | 5586 |
| Protein | 5562 |
| Ligand | 2 |
| Solvent | 22 |
| Total | 5586 |
| R.m.s. deviations | |
| Bonds (A) | 0.008 |
| Angles (°) | 1.06 |
| Average B factors (A2) | 42.54 |
| Protein | 42.59 |
| Ligand | 44.10 |
| Solvent 31.21 | |
| Ramachandran plot | |
| Most favoured (%) | 93.83 |
| Allowed (%) | 5.31 |

Results: For Kinetic characterization of high-purity HsADA1: the inventors expressed a codon-optimized HsADA1 gene, modified to append a C-terminal TEV protease site and hexahistidine tags, in E. coli T7 Express. SDS-PAGE analysis shows HsADA1 is >99% pure after nickel affinity chromatography, with an expected decrease in MW after TEV-cleavage from 43.5 kDa to 41.6 kDa. Fractionation of pure cleaved HsADA1 by size exclusion chromatography confirms HsADA1 is predominantly monomeric in solution, with only a small dimeric HsADA1 population. Expression yields range from 10 to 15 mg protein per liter at 95% purity or greater following affinity chromatography, similar to that obtained for MmADA.

The inventors evaluated HsADA1 kinetics with its preferred substrate, adenosine. Non-linear regression analysis reveals catalytic parameters (kcat=93.74±1.96 s-1 and kM=13.70±1.04 μM, approximately kcat/kM=7.15×106 M-is-1), similar to the reported activities of the BtADA (kcat=385 s-1 and kM=43. tM, kcat/kM=8.95×106 M-ls-1), and MmADA (kcat=2405−1 and kM=21. tM, kcat/kM=1.14×107 M-ls-1) and nearly two orders of magnitude higher than for HsADA2 (kcat=88 s-1 and kM=2 mM). Prior characterization of recombinant HsADA1 catalytic activity (kcat=1905−1 and KM=26. tM, kcat/KM=7.31×106 M-ls-1) is in agreement with results disclosed herein. The activity of tagged (kcat=94.55±3.485−1 and KM=12.41±1.60 μM kcat/KM=7.62×106 M-ls-1) and cleaved HsADA1 are nearly identical, and its thermal stability is similarly unaffected (Tm before removal=61.7±0° C. (n=3), Tm after cleavage=60.1±0° C. (n=3)). Thus, the hexahistidine tag and its removal does not interfere with catalysis or stability. Reactions conducted in a pH range between 3.0 and 10.8 demonstrate broad activity, with optimal pH values for HsADA1 degradation of adenosine at physiological pH range between 6.0 and 8.0.

Relating to the overall structure of holo HsADA1: the HsADA1 structure was solved to 2.6 A resolution by molecular replacement (see Table 1). HsADA1 adopts the expected a/(3-barrel architecture and TIM-barrel topology. The two copies of HsADA1 in the asymmetric unit are indistinguishable (RMSD=0.34 A) except for minor loop configurations. In addition, the calculated contact surface area between these two polypeptides is low (−600 A2), in line with chromatographic results indicating that HsADA1 is predominantly monomeric (see FIG. 20A). Both monomers in the asymmetric unit have a similarly organized active site with tightly bound Zn2+ cofactor, expected to lower the pKa of water to facilitate catalysis.

Structural comparisons of holo HsADA1 reveal that it adopts a closed conformation. When they are unbound by substrate, mammalian adenosine deaminase enzymes are thought to take on an ‘open’ conformation, characterized by the location of the L58-F65 a-helix and L182-D185 loop that make up a structural gate leading from the surface to the active site. Substrate binding is expected to be accompanied by a shift of the a-helix towards the loop, reducing access to the active site as the enzymes takes on the ‘closed’ conformation. Several lines of data support the conclusion that holo HsADA1 adopts an unexpected, closed conformation. First, comparison of holo HsADA1 to the 2DA/Ni2+ bound structure (PDB code 3IAR, RMSD=0.37 A), reveals near perfect overlap in the conformation of their structural gates. The Nit′ ion, which replaces Zn2′, would be expected to abolish catalytic activity, though the coordination environment for Nit′ and Zn2 appear similar. The main difference between holo HsADA1 to the 2DA/Ni2+ bound structure is that no electron density was observed for amino acids 354 to 364 in holo HsADA1, distal to the active site. In the 2DA/Ni2+ bound structure, this is a helix. These residues appear to have been proteolyzed prior to crystallization, as they appear incompatible with the crystal lattice. The tag does not affect activity (kcat=104±1.76 s-1 and kM=23±1.32 μM, kcat/kM=4.53×106 M-is-1) (see FIG. 13A). These terminal residues are absent in MmADA1, are not visible in BtADA structures available to date (not shown), and poorly predicted by Alphafold. Second, in comparison with holo BtADA1 (PDB: 1VFL), which takes the canonical open conformation, the L58-F65 helix in holo HsADA1 tilts 2-3.5 A to shrink the opening of the structural gate. Third, the inventors compared holo HsADA1 to structures of BtADA1 and MmADA1 bound to inhibitors that stabilize either the closed (MmADA1 in complex with 2′-deoxycoformycin (DCF; PDB code 1A4L) or 1-deaza-adenosine (1-DAA; PDB code 1ADD)) or open enzyme conformation (BtADA1 in complex with EHNA (PDB code 2Z7G) and FR235380 (PDB code 1QXL)). The structural gate of holo HsADA1 shows clear alignment with the closed conformation of DCF or 1-DAA bound enzyme, and a noticeable shift away from the open conformation seen in EHNA and FR232580 complexed enzymes. Finally, the structural gate configuration of holo HsADA1 is similar to that of holo MmADA1 (PDB code 3MVI), which has a bound glycerol near the gate (but see below).

Analysis of crystal contacts for holo HsADA1, MmADA and BtADA rules out possible crystallographic artifact of closed conformation. Next, the inventors considered the role of crystal packing on the conformation of the structural gate configuration across the three orthologous holo enzyme structures. In all three structures, there is a crystal contact near the structural gate helix (L58-F65), employing Y67. The side chain of Y67 forms contacts with different residues in crystallographically-related protein copies. In holo HsADA1, the interaction is with main chain carbonyl oxygens of P354 and P355, in holo MmADA, it is a water-mediated contact to E345, and in holo BtADA, the interaction is with K207. In HsADA1 and MmADA, there are no other contacts within in the structural gate helix. By contrast, in holo BtADA, there is a crystal contact within the structural gate helix. The side chain of D61 forms a contact with S207 from a crystallographically-related molecule. The open conformation in BtADA may be a possible crystallographic artifact, as this contact may artificially induce or stabilize the open conformation. In the available closed conformation of BtADA observed in the structure in complex with 6-hydroxyl-1,6-dihydropurine (HDPR, PDB code 1KRM), D61 does not make a crystal contact. Taken together, crystal packing in holo HsADA1 is consistent with closed conformations of other orthologs.

Relating to analysis of holo HsADA1 substrate and receptor binding sites: cavity analysis of holo HsADA1 highlights the substrate binding pocket and extent of its accessibility through the ‘closed’ catalytic gate. There is a pocket at the equivalent site for substrate or inhibitor binding in other mammalian ADA1 enzymes. In support of the notion that a conformational change to a more open state is required to access the substrate binding site, computational docking fails to find a pose in the active site below the closed gate, whereas the equivalent docking to BtADA1, in an open conformation, is successful (not shown).

Functionally, the conformation of the structural gate has been speculated to be important for HsADA1 binding to the adenosine receptors AJAR and A2AAR. The residues implicated in AR binding, L58 to 172 and A184 to 1188, rim two sides of the entrance to the HsADA1 active site, overlapping with the structural gate. By contrast, HsADA1 residues implicated in its interaction with the costimulatory CD26 molecule, P126 to D143, are located on a helix remote from the structural gate on the opposite face of the protein. Thus, HsADA1 should be able to simultaneous interact with multiple binding partners, as has been previously proposed.

Comparing of HsADA1 and HsADA2, HsADA2 has a much higher kM for adenosine than HsADA1 (mM versus low μM), is larger, and forms a homodimer. HsADA2 dimerization occurs primarily through interactions between regions that are not conserved in HsADA1. The overall TIM barrel fold and coordination with the Zn2′ cofactor are conserved in both enzymes (r.m.s.d.=4.7 A) despite low primary sequence homology (% identity=22%). One relevant loop with a vastly different conformation for each enzyme consists of amino acids 107 to 126 in HsADA1 (homologous to amino acids 221 to 235 in HsADA2), referred to in prior work as the ‘b2-a2’ loop. In HsADA1, ‘b2-a2’ participates in hydrophobic contacts with the L58-F65 helix of the active site gate, but these interactions are precluded in the configuration of ‘b2-a2’ present in HsADA2, which takes the same ‘open’ conformation as a holo enzyme or when bound by coformycin (CF). Thus, the conformation of ‘b2-a2’ may affect open-closed conformations of the structural gate in human ADAs.

Herein is presented the biochemical characterization and crystal structure of holo HsADA1, which exhibits pleiotropic effects on immune signaling by hydrolysis of its substrate adenosine and by binding to CD26 and ARs. The inventors overcame prior limitations of preparative expression in E. coli by using an E. coli codon-optimized gene for HsADA1 and a robust protein production strain. This process resulted in impressive expression yields to enable direct characterization of this biomedically important enzyme (see FIGS. 19, 20A, 20B, 21A, 21B, and 13B).

For 25 years, insights into HsADA1 structure and function have been derived from studies of close homologs MmADA (83% identical to HsADA1) and BtADA (89% identical to HsADA1). Early observations based on inhibitor-bound MmADA and BtADA structures noted that there was no entry point to the active site wide enough to allow inhibitor access, implying that a mammalian ADA enzymes convert from an “open” conformation in the holo form to a closed state upon binding substrates led to the conclusion that holo MmADA adopted a “closed” conformation because of a fortuitously bound glycerol molecule; the open conformation of MmADA has not been experimentally observed.

The structure of holo HsADA1 disclosed herein, which adopts the “closed” conformation common to holo MmADA, raises new doubts that the “open” structural gate conformation is stable across mammalian ADAs. Crystal packing analysis indicates that a structural gate helix residue, D61, is involved in a crystal contact in holo BtADA, but not in holo HsADA1, holo MmADA, or HDPR-bound BtADA1. In addition, Alphafold predicts holo BtADA to be in the closed conformation. The closed conformation appears readily trapped crystallographically for MmADA and HsADA1, even though the opportunity for a crystal contact employing D61, is available across orthologs. The role of crystal contacts on the observed conformation of mammalian ADA enzymes should be explored further. For example, the open conformation seen for EHNA and FR235380 bound to BtADA may be because of lattice constraints, and only the stabilizing interactions endowed by HDPR binding can disfavor the crystal contact with D61 to stabilize the −4 A shifted closed conformation. In this context, the resemblance of holo HsADA1 to holo MmADA indicates that no conformational change occurs upon DCF or 1-DAA binding. These observations should prompt direct experiments with MmADA and HsADA1 to determine whether EHNA or FR235380 can stabilize an open state, including, critically, in solution.

Alternatively, BtADA may be inherently different from MmADA and HsADA1. First, although the three homologous sequences are >80% identical, including all residues that line the active site, the protein sequence of the holo BtADA structure differs by 8 residues compared to the protein sequence deposited to Uniprot, of which 4 positions have been substituted by the residue in HsADA1. The origin of these substitutions is not clear but may originate from challenges in expression or purification of BtADA1 for structure determination or from natural genetic diversity with the Bos taurus species. Perhaps the conformations adopted by BtADA are endowed by these residue substitutions. Second, holo HsADA2 adopts an open conformation. However, should be noted the sequence similarity with HsADA2 (23% identity for BtADA, 22% for HsADA1) is low, the analogous gating helix in HsADA2 is longer, and no change to a closed conformation is observed upon CF binding. Finally, catalytic pH profiles differ between HsADA1, BtADA, and HsADA2; kinetic parameters as a function of pH have not been reported for MmADA. Hydrolysis of adenosine involves acid-base catalysis mediated by the Zn2′ cofactor, a coordinated water molecule, and active site residues Glu214 and His235. HsADA1 activity as a function of pH resembles the general trend of highest activity −pH 6-8 reported for BtADA, but HsADA1 is more active than BtADA1 at higher pH values, as BtADA retains 20% activity at pH 8.4, whereas HsADA1 retains −50% activity at pH 9.2 (see FIGS. 19, 20A, 20B, 21A, 21B, and 13B). In this vein, HsADA2 exhibits a pH maximum at −pH 6.8, and the range in which HsADA2 is active is less broad than for HsADA1. Perhaps the more sequestered active site of HsADA1 mutes the effect of solvent by limiting pKa perturbation of ionizable catalytic residues like His235 and Glu214 as a function of pH, though it is noted that the use of different assay methods across these studies could account for some discrepancies. In addition, the kM of BtADA (kM=43 μM) is slightly higher than that of HsADA1 (measured to between 13-26 μM) or MmADA (21 μM), suggesting existence of an energetic barrier associated with the conformational change needed for substrate binding.

Even though HsADA1 and MmADA do not appear to readily adopt the open conformation captured for BtADA, there is still a need for a conformational change to allow substrate to enter the active site. Two options are likely based on other enzymes: 1) dynamics and 2) allosteric regulation. Supporting a role for dynamics, open and closed conformations have been extensively reported upon for a canonical TIM barrel enzyme. Upon binding, the substrate is trapped within a hydrophobic cage to facilitate catalysis. Following conversion, the product is released by the movement of peptide loops to an open conformation. Therefore, the open conformation is a low-population excited state stabilized by the crystal lattice that is not accessed crystallographically in HsADA1. Alternatively, BtADA has been shown to be allosterically modulated the mixed inhibition mechanism of 1,3-dinitrobenzene, though it is unknown if allosteric modulation could impose a change in the conformational of BtADA's structural gate. Notably, the C-terminal residues common to BtADA and HsADA1 have only observed crystallographically in the 2DA/Ni2′ HsADA1 structure. Perhaps there are long range motions that propagate from these distant regions of the protein that are relevant to allostery.

The conformational state of HsADA1 is crucial in its role as an immunomodulator, as its binding to ARs has been shown to amplify both agonistic and antagonistic signaling outputs. The residues comprising the HsADA1 structural gate and predicted AR binding residues overlap, and prior studies have inferred or presumed that only the open conformation of HsADA1 can interact with ARs, which the structure disclosed herein calls into question. First, holo HsADA1 has been shown to bind and amplify signaling through AJAR and A2AAR, which the inventors have identified as occurring in a closed conformation. In addition, the now-unlikely notion that MmADA converts from an open to a closed conformation upon DCF binding was used to rationalize why DCF incubation abrogates the ability of HsADA1 to amplify signaling through AJAR and A2AAR. Namely, HsADA1 was assumed to undergo the same open to closed conformational change (upon DCF binding, such that its AR binding site would be distorted. Since a shift of the structural gate is highly unlikely, other constraints must be responsible for DCF-complexed HsADA1 lack of binding to AJAR and A2AAR. Further, HsADA1 has been shown to be able fine-tune germinal center and circulating follicular T cell helper programs (cTfh2-17) to improve downstream antibody production, in part due to its abilities to (1) degrade adenosine and (2) amplify signaling through AJAR or A3AR. DCF had no effect on HsADA1's impact on cTfh2-17 cells, suggesting that binding of specific receptors may not be impacted, perhaps because no conformational change occurs.

Conversely, EHNA, thought to stabilize an open conformation based on BtADA1 structures, abrogated the effect of HsADA1 on cTfh2-17 cells. Therefore, the structure disclosed herein should prompt new experiments to explore the molecular explanation for the effect of ligand complexation on HsADA1 binding to ARs.

In sum, structures of HsADA1, BtADA, and MmADA converge on the requirement for a conformational change to allow for substrate binding and catalysis, but details differ across orthologs. The finding that holo HsADA1 adopts a closed conformation helps reinterpret the conformation of holo MmADA and indicates that there is an unanticipated barrier to opening the substrate gate. There is a possibility that conclusions drawn from conformational changes in BtADA, which was captured in an open conformation in the absence of inhibitor in the active site, may eventually serve as a cautionary tale for the use of homologs to address functional questions of difficult biomedically-relevant targets like HsADA1. Future studies can now focus on alternative mechanisms by which conformational changes may be triggered in HsADA1 and the relevance of these changes for interactions with binding partners like ARs.

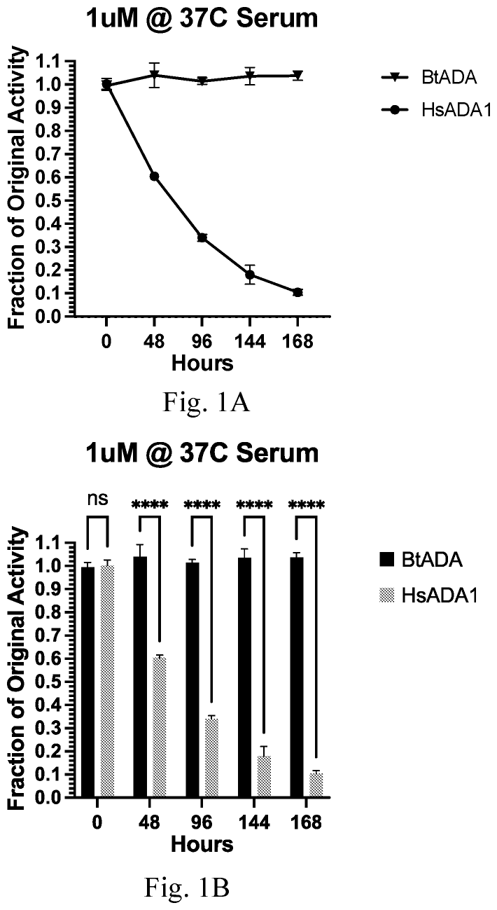

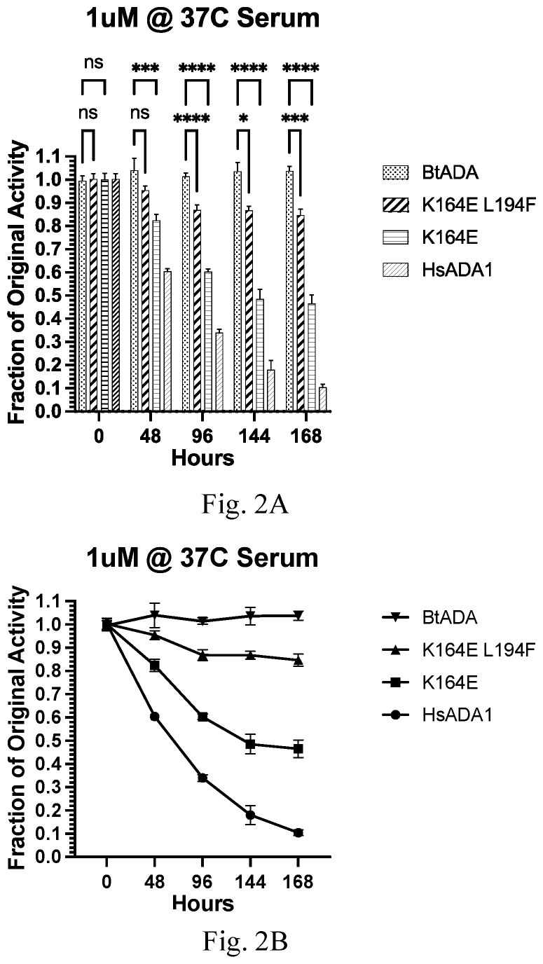

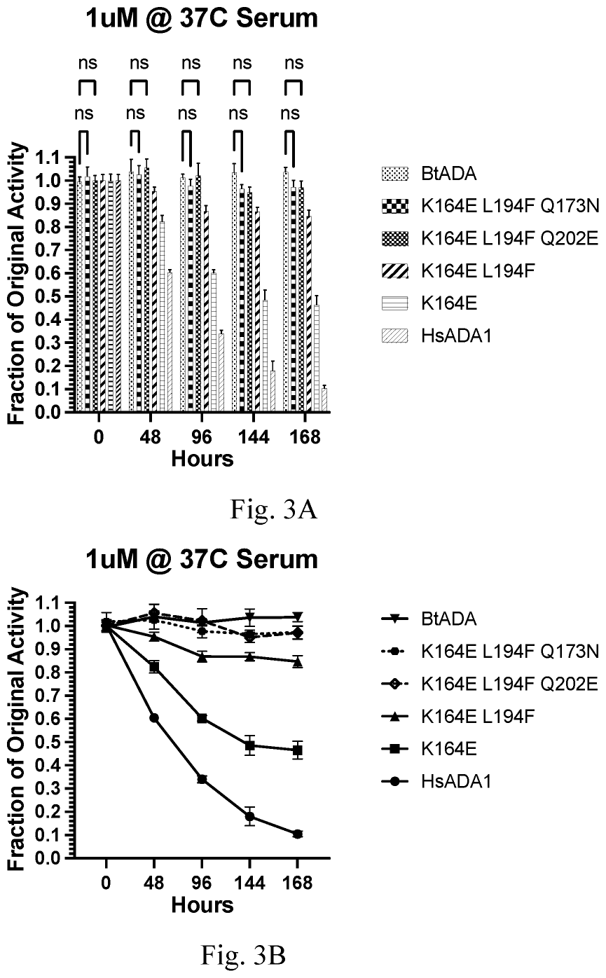

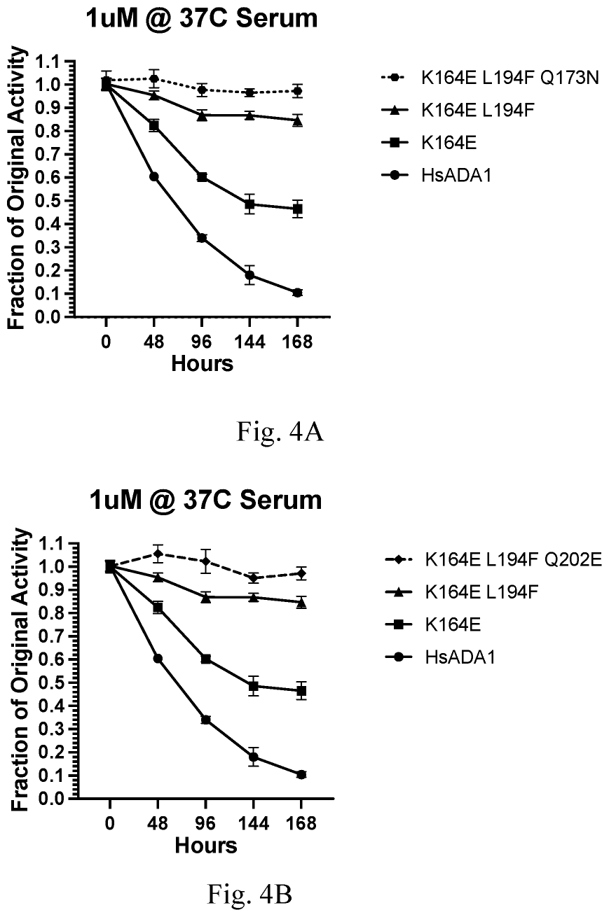

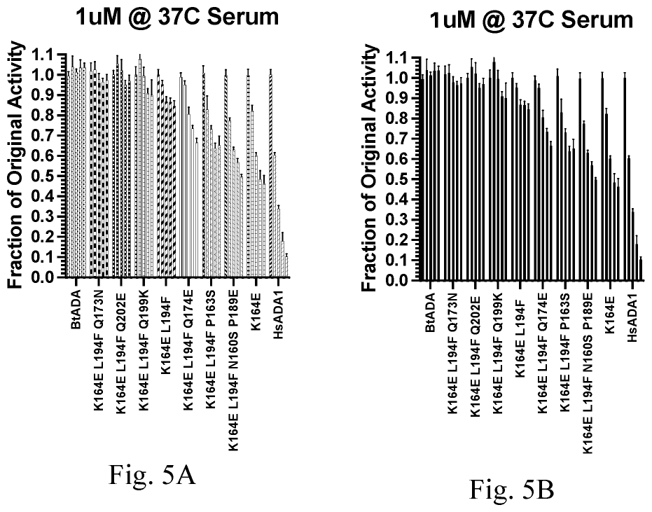

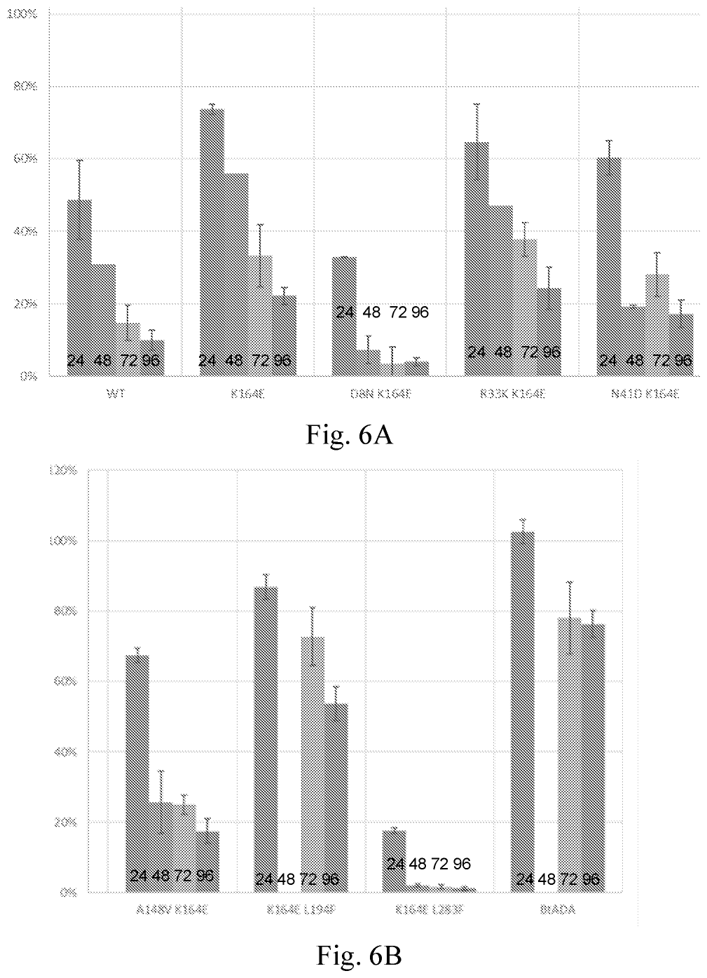

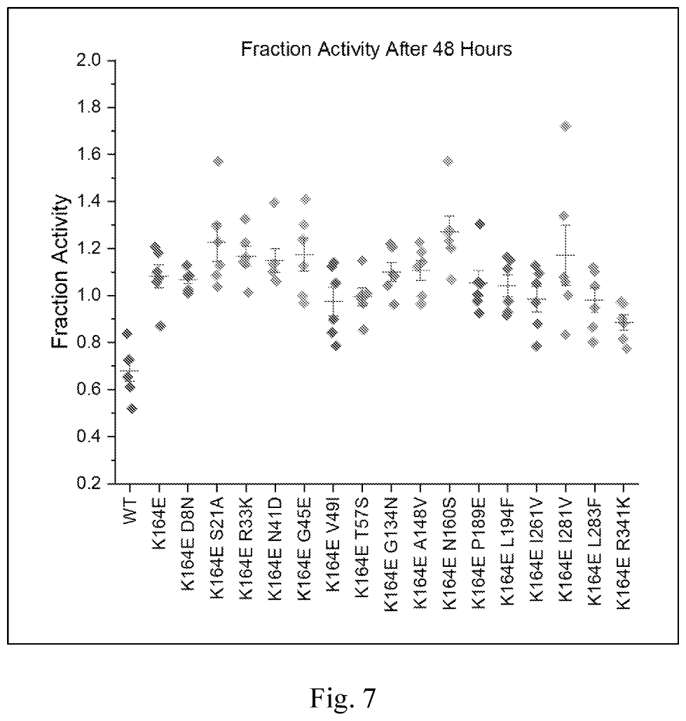

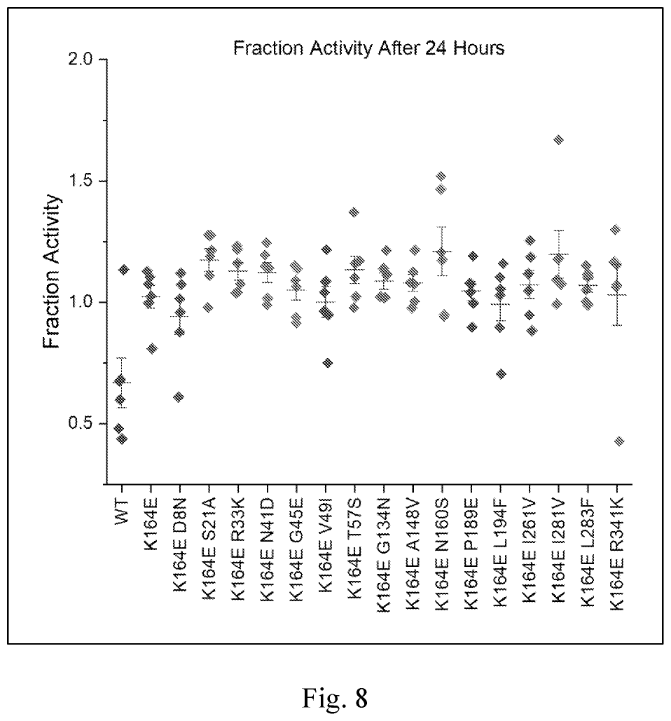

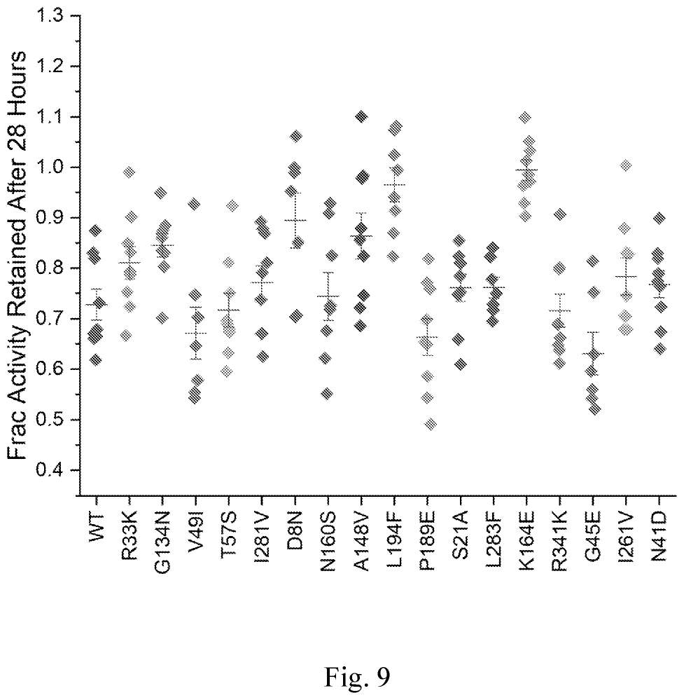

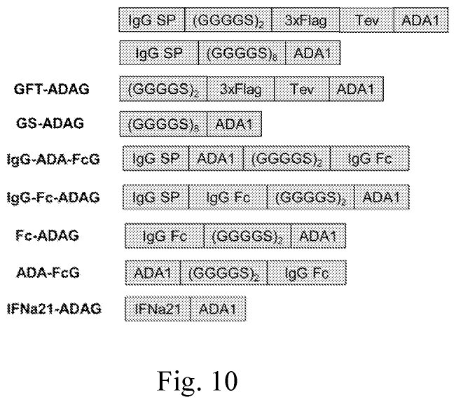

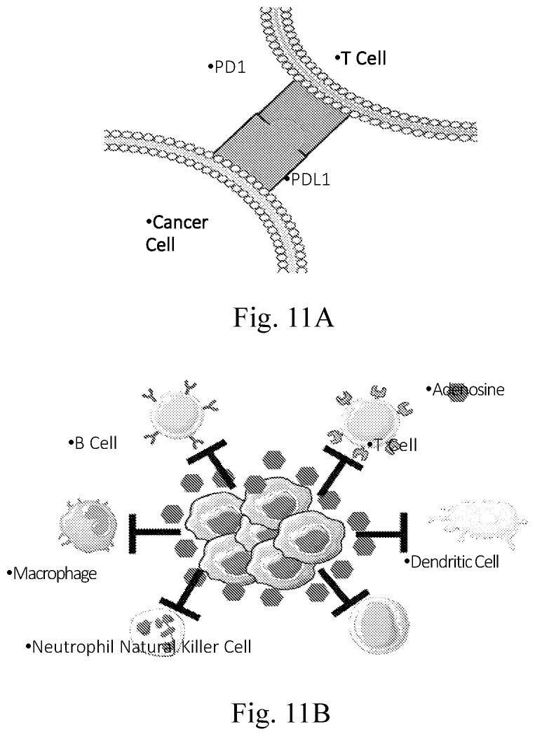

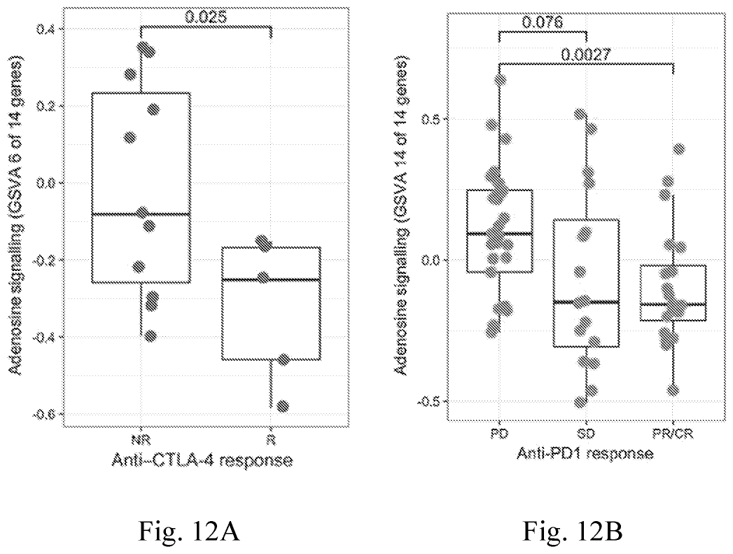

Combinations of mutations in amino acid positions described herein are shown in FIGS. 1-10. For example, combinations of (i) K164E, L194F, and Q199K; (ii) K164E, L194F, and Q173N; or (iii) K164E, L194F, and Q202E were shown to have increased stability relative to wildtype HsADA1, and comparable to BtADA.

### Example 4—Method of Making the ADA1 Variants

The nucleic acid molecule encoding the HsADA1 variant can be present in a vector (e.g., a viral vector or a plasmid) or in an expression cassette. The vector or expression cassette can be present in a cell, such as a prokaryotic cell or eukaryotic cell. The vector or expression cassette may be permanently or transiently integrated into the host cell genome, or may be maintained extrachromasomally by suitable methods such as selection pressure. The vector or expression cassette can include a strong promoter operably linked to the nucleic acid molecule encoding the HsADA1 variant. The promoter can be inducible or constitutive. Nonlimiting examples of prokaryotic cells include cells suitable for expression of heterologous proteins, such as for example and not limitation, Escherichia coli. Nonlimiting examples of eukaryotic cells include mammalian cells, such as immune cells and non-immune cells. Nonlimiting examples of immune cells include T cells, CAR T cells, B cells, natural killer (NK) cells, and neutrophils. Nonlimiting examples of non-immune cells can include cell lines that are suitable for expression of heterologous proteins, such as for example and not limitation, Chinese hamster ovary (CHO) cells, HEK293T Cells, and Expi293T cells.

The cell containing the nucleic acid molecule encoding the HsADA1 variant is cultured under conditions suitable to express the HsADA1 variant, such as for example and not limitation, inducing the expression of the HsADA1 variant, and expanding the population of cells containing the nucleic acid molecule encoding the HsADA1 variant. The cells can be lysed by any appropriate methods, and the HsADA1 variant can be collected or isolated from the cell lysate by any appropriate methods.

For example, a plasmid containing HsADA1 expressed from a strong IPTG-inducible promoter is transformed into E. coli, and maintained using appropriate selective pressure (e.g., antibiotics). After sufficient growth, the E. coli cultures containing the plasmid are placed in an ice-water slurry and moved to a 4° C. cold room for 30 minutes, followed by addition of 3% v/v ethanol, and each culture is induced to a final concentration of 0.5 mM IPTG. The flasks are then kept for 48-56 hours at 15° C., shaking at 200 RPM, to induce His-tagged HsADA1 protein expression. Induced cultures are harvested by centrifugation at 3,400×g and 4° C. for 30 minutes and stored at −80° C. The E. coli cells can then be lysed and the cell lysates applied to a column capable of trapping His-tagged proteins. The His-tag can be removed by any method known in the art, including a TEV-protease based system as described herein.

### Example 5—Method of Treating a Cancer or Tumor with the ADA1-Containing Compositions

A therapeutically effective amount of an ADA1 variant or an ADA1-containing composition as described herein is administered to a subject in need thereof, to treat a cancer or tumor in the subject. The ADA1 variant can be present with a pharmaceutically acceptable carrier and/or excipient. The ADA1-containing composition can include a pharmaceutically acceptable carrier and/or excipient. The route of administration can be any suitable route, such as intratumoral, peritumoral, intradermal, subcutaneous, intravenous, or intraperitoneal. The subject can be a mammal, such as a human or a veterinary animal. The ADA1 variant or ADA1-containing composition can be administered with one or more cancer immunotherapies, adenosine depleting therapies, or ADA1 enzyme replacement therapies as described herein.

### Example 6—Method of Treating ADA-SCID with the ADA1-Containing Compositions

A therapeutically effective amount of an ADA1 variant or an ADA1-containing composition as described herein is administered to a subject in need thereof, to treat ADA-SCID in the subject. The ADA1 variant can be present with a pharmaceutically acceptable carrier and/or excipient. The ADA1-containing composition can include a pharmaceutically acceptable carrier and/or excipient. The route of administration can be any suitable route, such as intratumoral, peritumoral, intradermal, subcutaneous, intravenous, or intraperitoneal. The subject can be a mammal, such as a human or a veterinary animal. The ADA1 variant or ADA1-containing composition can be administered with one or more ADA1 replacement therapies as described herein.

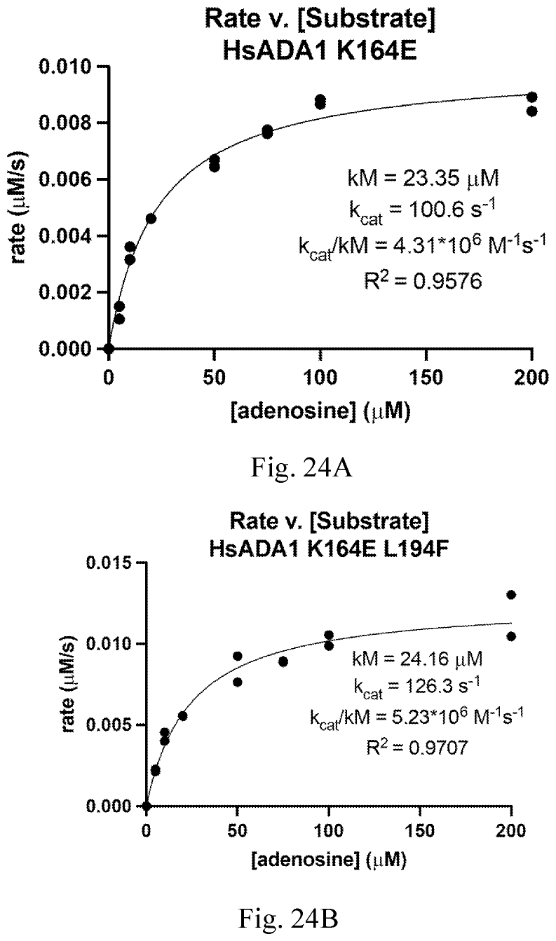

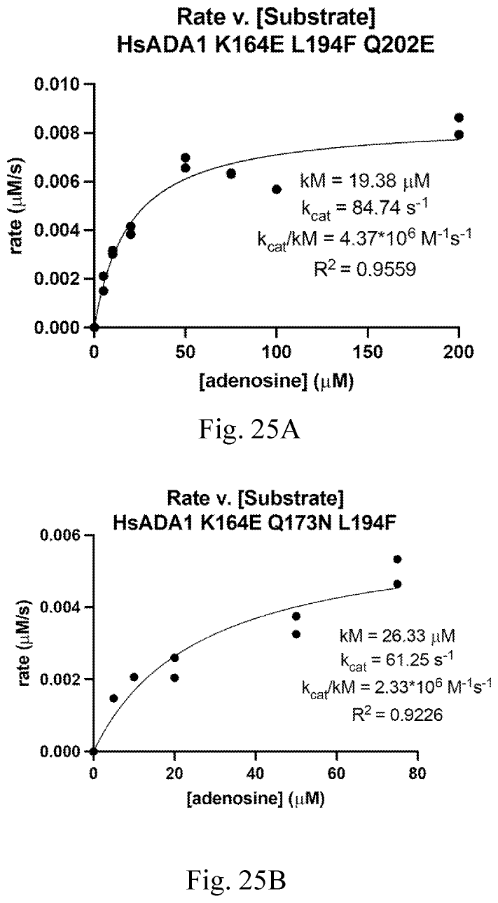

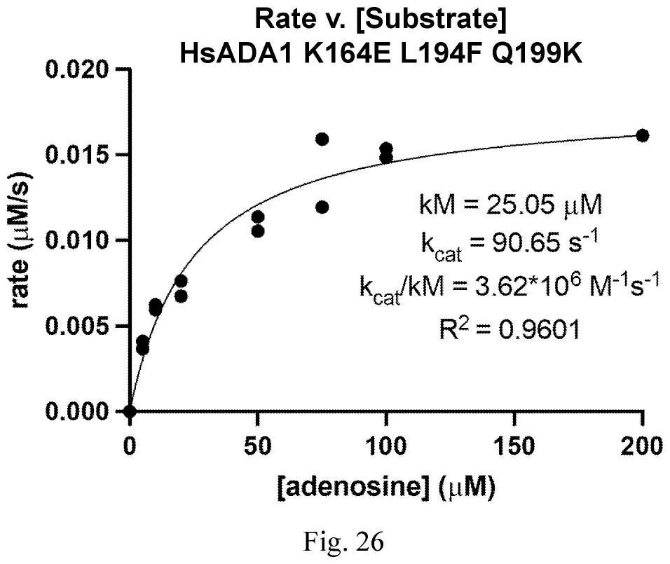

Kinetics for HsADA1 mutants are in FIGS. 24A-26

Constructs Made with Wildtype HsADA1 while Optimizing Expression and Purification:

HsADA 1-GG-6His-GG:
(Seq ID No. 1)
ATGGCTCAAACTCCGGCCTTCGACAAGCCGAAAGTCGAACTCCATGTACATCTTG
ATGGAAGTATAAAACCGGAGACGATCCTGTATTATGGGCGCCGTCGCGGTATTG
CTTTACCTGCCAATACCGCAGAAGGTCTGCTGAACGTTATCGGCATGGACAAACC
CCTCACCCTTCCGGATTTTCTGGCTAAGTTCGATTATTACATGCCGGCGATTGCTG
GTTGTCGTGAAGCCATCAAACGCATTGCTTATGAGTTTGTTGAAATGAAAGCGAA
AGAAGGCGTGGTCTACGTTGAAGTACGTTATTCTCCTCACCTGTTAGCTAATTCC
AAGGTGGAGCCAATCCCGTGGAACCAAGCCGAAGGAGATCTGACACCAGACGA
AGTTGTCGCACTGGTGGGCCAGGGTCTCCAGGAGGGGGAACGCGACTTCGGCGT
GAAAGCCCGTTCGATTCTTTGCTGTATGCGCCATCAGCCCAATTGGAGCCCGAAA
GTAGTAGAGTTGTGCAAAAAGTACCAACAGCAAACTGTCGTTGCAATCGATCTG
GCGGGTGACGAAACGATTCCGGGGAGTTCTTTACTGCCAGGACACGTGCAGGCC
TATCAGGAAGCGGTGAAATCCGGCATCCATCGTACGGTTCACGCAGGTGAGGTC
GGCTCAGCCGAAGTGGTTAAAGAGGCGGTGGATATTCTGAAAACCGAACGCCTC
GGCCACGGTTACCACACCTTGGAAGACCAAGCACTGTATAACCGTCTGCGCCAG
GAGAATATGCATTTTGAAATCTGCCCCTGGAGCAGTTACTTAACTGGCGCCTGGA
AACCTGACACAGAACATGCAGTGATTCGTTTGAAAAACGATCAGGCGAATTATT
CTCTTAACACGGATGATCCGCTGATATTCAAATCCACCCTGGATACCGACTACCA
GATGACCAAACGCGATATGGGGTTTACAGAGGAAGAATTTAAACGTCTGAATAT
TAACGCGGCGAAATCAAGCTTTTTGCCAGAGGATGAAAAGCGCGAACTTTTAGA
TCTGCTGTATAAGGCGTACGGTATGCCGCCTTCGGCAAGCGCGGGCCAGAACTTG
GGCGGTCATCATCACCACCATCACGGCGGCTAA
(Seq ID No. 2)
MAQTPAFDKPKVELHVHLDGSIKPETILYYGRRRGIALPANTAEGLLN
VIGMDKPLTLPDFLAKFDYYMPAIAGCREAIKRIAYEFVEMKAKEGVVYVEVRYSPH
LLANSKVEPIPWNQAEGDLTPDEVVALVGQGLQEGERDFGVKARSILCCMRHQPNW
SPKVVELCKKYQQQTVVAIDLAGDETIPGSSLLPGHVQAYQEAVKSGIHRTVHAGEVGS
AEVVKEAVDILKTERLGHGYHTLEDQALYNRLRQENMHFEICPWSSYLTGAWKPDTEH
AVIRLKNDQANYSLNTDDPLIFKSTLDTDYQMTKRDMGFTEEEFKRLNINAAKSSFLPE
DEKRELLDLLYKAYGMPPSASAGQNLGGHHHHHHGG
HsADA1-GG-6His-GG-8His-GG:
(Seq ID No. 3)
ATGGCTCAAACTCCGGCCTTCGACAAGCCGAAAGTCGAACTCCATGTACATCTTGAT
GGAAGTATAAAACCGGAGACGATCCTGTATTATGGGCGCCGTCGCGGTATTGCTTTA
CCTGCCAATACCGCAGAAGGTCTGCTGAACGTTATCGGCATGGACAAACCCCTCACC
CTTCCGGATTTTCTGGCTAAGTTCGATTATTACATGCCGGCGATTGCTGGTTGTCGTG
AAGCCATCAAACGCATTGCTTATGAGTTTGTTGAAATGAAAGCGAAAGAAGGCGTG
GTCTACGTTGAAGTACGTTATTCTCCTCACCTGTTAGCTAATTCCAAGGTGGAGCCA
ATCCCGTGGAACCAAGCCGAAGGAGATCTGACACCAGACGAAGTTGTCGCACTGGT
GGGCCAGGGTCTCCAGGAGGGGGAACGCGACTTCGGCGTGAAAGCCCGTTCGATTC
TTTGCTGTATGCGCCATCAGCCCAATTGGAGCCCGAAAGTAGTAGAGTIGTGCAAAA
AGTACCAACAGCAAACTGTCGTTGCAATCGATCTGGCGGGTGACGAAACGATTCCG
GGGAGTTCTTTACTGCCAGGACACGTGCAGGCCTATCAGGAAGCGGTGAAATCCGG
CATCCATCGTACGGTTCACGCAGGTGAGGTCGGCTCAGCCGAAGTGGTTAAAGAGG
CGGTGGATATTCTGAAAACCGAACGCCTCGGCCACGGTTACCACACCTTGGAAGAC
CAAGCACTGTATAACCGTCTGCGCCAGGAGAATATGCATTTTGAAATCTGCCCCTGG
AGCAGTTACTTAACTGGCGCCTGGAAACCTGACACAGAACATGCAGTGATTCGTTTG
AAAAACGATCAGGCGAATTATTCTCTTAACACGGATGATCCGCTGATATTCAAATCC
ACCCTGGATACCGACTACCAGATGACCAAACGCGATATGGGGTTTACAGAGGAAGA
ATTTAAACGTCTGAATATTAACGCGGCGAAATCAAGCTTTTTGCCAGAGGATGAAAA
GCGCGAACTTTTAGATCTGCTGTATAAGGCGTACGGTATGCCGCCTTCGGCAAGCGC
GGGCCAGAACTTGGGCGGTCATCATCACCACCATCACGGCGGCCATCACCATCACC
ATCACCATCACGGGGGTTAA
(Seq ID No. 4)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHGG
HsADA1 (codonopt 1)-GG-6His-GG-8His-GG:
(Seq ID No. 5)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACGGGG
GTTAA
(Seq ID No. 6)
MAQTPAFDKPKVELHVHLDGSIKPETILYYGRRRGIALPANTAEGLLN
VIGMDKPLTLPDFLAKFDYYMPAIAGCREAIKRIAYEFVEMKAKEGVVYVEVRYSPHLL
ANSKVEPIPWNQAEGDLTPDEVVALVGQGLQEGERDFGVKARSILCCMRHQPNWSPKV
VELCKKYQQQTVVAIDLAGDETIPGSSLLPGHVQAYQEAVKSGIHRTVHAGEVGSAEVV
KEAVDILKTERLGHGYHTLEDQALYNRLRQENMHFEICPWSSYLTGAWKPDTEHAVIR
LKNDQANYSLNTDDPLIFKSTLDTDYQMTKRDMGFTEEEFKRLNINAAKSSFLPEDEKR
ELLDLLYKAYGMPPSASAGQNLGGHHHHHHGGHHHHHHHHGG
HsADA1 (codonopt 1)-GG-6His-GG-10His-GG:
(Seq ID No. 7)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 8)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1 (codonopt 1)-GG-6His-GG:
(Seq ID No. 9)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCTAA
(Seq ID No. 10)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGG
HsADA1 (codonopt1)-GG-6His-GG-6His-GG:
(Seq ID No. 11)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACGGGGGTTAA
(Seq ID No. 12)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHGG
HsADA1 (codonopt 1)-TEV-GG-6His-GG-6His:
(Seq ID No. 13)
ATGGCTCAAACTCCGGCCTTCGACAAGCCGAAAGTCGAACTCCATGTACATCTTG
ATGGAAGTATAAAACCGGAGACGATCCTGTATTATGGGCGCCGTCGCGGTATTGCTTT
ACCTGCCAATACCGCAGAAGGTCTGCTGAACGTTATCGGCATGGACAAACCCCTCAC
CCTTCCGGATTTTCTGGCTAAGTTCGATTATTACATGCCGGCGATTGCTGGTTGTCGTG
AAGCCATCAAACGCATTGCTTATGAGTTTGTTGAAATGAAAGCGAAAGAAGGCGTGG
TCTACGTTGAAGTACGTTATTCTCCTCACCTGTTAGCTAATTCCAAGGTGGAGCCAATC
CCGTGGAACCAAGCCGAAGGAGATCTGACACCAGACGAAGTTGTCGCACTGGTGGG
CCAGGGTCTCCAGGAGGGGGAACGCGACTTCGGCGTGAAAGCCCGTTCGATTCTTTG
CTGTATGCGCCATCAGCCCAATTGGAGCCCGAAAGTAGTAGAGTTGTGCAAAAAGTA
CCAACAGCAAACTGTCGTTGCAATCGATCTGGCGGGTGACGAAACGATTCCGGGGAG
TTCTTTACTGCCAGGACACGTGCAGGCCTATCAGGAAGCGGTGAAATCCGGCATCCAT
CGTACGGTTCACGCAGGTGAGGTCGGCTCAGCCGAAGTGGTTAAAGAGGCGGTGGA
TATTCTGAAAACCGAACGCCTCGGCCACGGTTACCACACCTTGGAAGACCAAGCACT
GTATAACCGTCTGCGCCAGGAGAATATGCATTTTGAAATCTGCCCCTGGAGCAGTTAC
TTAACTGGCGCCTGGAAACCTGACACAGAACATGCAGTGATTCGTTTGAAAAACGAT
CAGGCGAATTATTCTCTTAACACGGATGATCCGCTGATATTCAAATCCACCCTGGATAC
CGACTACCAGATGACCAAACGCGATATGGGGTTTACAGAGGAAGAATTTAAACGTCT
GAATATTAACGCGGCGAAATCAAGCTTTTTGCCAGAGGATGAAAAGCGCGAACTTTT
AGATCTGCTGTATAAGGCGTACGGTATGCCGCCTTCGGCAAGCGCGGGCCAGAACTT
GGAGAACCTGTACTTCCAATCCGGCGGTCATCATCACCACCATCACGGCGGCCACCA
CCACCACCACCACTGA
(Seq ID No. 14)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLENLYFQSGGHHHHHHGGHHHHHH
HsADA1 (codonopt1)-TEV-GG-6His-GG-10His-GG:
(Seq ID No. 15)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GAGAACCTGTACTTCCAATCCGGCGGTCATCATCACCACCATCACGGCGGCCATCACC
ATCACCATCACCATCACCATCACGGGGGTTAA
(Seq ID No. 16)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLENLYFQSGGHHHHHHGGHHHHHHHHHHGG
HsADA1 (codonopt1):
(Seq ID No. 17)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
TAA
(Seq ID No. 18)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1 variants with mutations:
HsADA1 A148V (codonopt1):
(Seq ID No. 19)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGTTCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
TAA
(Seq ID No. 20)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKVRSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1 A148V (codonopt1)-GG-6His-GG-10His-GG:
(Seq ID No. 21)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGTTCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 22)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKVRSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1 A148V K164E (codonopt1):
(Seq ID No. 23)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGTTCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 24)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKVRSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1 A148V K164E (codonopt1)-GG-6His-GG-10His-GG:
(Seq ID No. 25)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGTTCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 26)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKVRSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1 C57S (codonopt 1):
(Seq ID No. 27)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTCTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 28)
MAQTPAFDKPKVELHVHLDGSIKPETILYYGRRRGIALPANTAEGLLNVIGM
DKPLTLPDFLAKFDYYMPAIAGSREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1 C57S (codonopt 1)-GG-6His-GG:
(Seq ID No. 29)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTCTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCTAA
(Seq ID No. 30)
MAQTPAFDKPKVELHVHLDGSIKPETILYYGRRRGIALPANTAEGLLNVIGM
DKPLTLPDFLAKFDYYMPAIAGSREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGG
HsADA1 D8N (codonopt1):
(Seq ID No. 31)
ATGGCTCAAACTCCGGCCTTCAATAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 32)
MAQTPAFNKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1D8N (codonopt1)-GG-6His-GG-10His-GG:
(Seq ID No. 33)
ATGGCTCAAACTCCGGCCTTCAATAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 34)
MAQTPAFNKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1D8NK164E (codonopt1):
(Seq ID No. 35)
ATGGCTCAAACTCCGGCCTTCAATAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 36)
MAQTPAFNKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1D8NK164E (codonopt1)-GG-6His-GG-10His-GG:
(Seq ID No. 37)
ATGGCTCAAACTCCGGCCTTCAATAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 38)
MAQTPAFNKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1D60A (codonopt1):
(Seq ID No. 39)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGCGTTTCTGGCTAAGTTCGATTATTACATGCCGGCGATTGCTGGTTGTCGTGAA
GCCATCAAACGCATTGCTTATGAGTTTGTTGAAATGAAAGCGAAAGAAGGCGTGGTC
TACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 40)
MAQTPAFDKPKVELHVHLDGSIKPETILYYGRRRGIALPANTAEGLLNVIGM
DKPLTLPAFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1D60A (codonopt1)-GG-6His-GG-10His-
(Seq ID No. 41)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGCGTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCCGAAAGTAGTAGAGTTGTGCA
AAAAGTACCAACAGCAAACTGTCGTTGCAATCGATCTGGCGGGTGACGAAACGATT
CCGGGGAGTTCTTTACTGCCAGGACACGTGCAGGCCTATCAGGAAGCGGTGAAATC
CGGCATCCATCGTACGGTTCACGCAGGTGAGGTCGGCTCAGCCGAAGTGGTTAAAG
AGGCGGTGGATATTCTGAAAACCGAACGCCTCGGCCACGGTTACCACACCTTGGAA
GACCAAGCACTGTATAACCGTCTGCGCCAGGAGAATATGCATTTTGAAATCTGCCCC
TGGAGCAGTTACTTAACTGGCGCCTGGAAACCTGACACAGAACATGCAGTGATTCGT
TTGAAAAACGATCAGGCGAATTATTCTCTTAACACGGATGATCCGCTGATATTCAAA
TCCACCCTGGATACCGACTACCAGATGACCAAACGCGATATGGGGTTTACAGAGGA
AGAATTTAAACGTCTGAATATTAACGCGGCGAAATCAAGCTTTTTGCCAGAGGATGA
AAAGCGCGAACTTTTAGATCTGCTGTATAAGGCGTACGGTATGCCGCCTTCGGCAAG
CGCGGGCCAGAACTTGGGCGGTCATCATCACCACCATCACGGCGGCCATCACCATC
ACCATCACCATCACCATCACGGGGGTTAA
(Seq ID No. 42)
MAQTPAFDKPKVELHVHLDGSIKPETILYYGRRRGIALPANTAEGLLNVIGM
DKPLTLPAFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1D185A (codonopt1):
(Seq ID No. 43)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGCTGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 44)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGAETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1D185A (codonopt1)-GG-6His-GG-10His-GG:
(Seq ID No. 45)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGCTGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 46)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGAETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1E217K (codonopt1):
(Seq ID No. 47)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTAAAGTCGGCTCAGCCGAAGTGGTTAAAGAGGCGGTGGATA
TTCTGAAAACCGAACGCCTCGGCCACGGTTACCACACCTTGGAAGACCAAGCACTGT
ATAACCGTCTGCGCCAGGAGAATATGCATTTTGAAATCTGCCCCTGGAGCAGTTACTT
AACTGGCGCCTGGAAACCTGACACAGAACATGCAGTGATTCGTTTGAAAAACGATCA
GGCGAATTATTCTCTTAACACGGATGATCCGCTGATATTCAAATCCACCCTGGATACCG
ACTACCAGATGACCAAACGCGATATGGGGTTTACAGAGGAAGAATTTAAACGTCTGA
ATATTAACGCGGCGAAATCAAGCTTTTTGCCAGAGGATGAAAAGCGCGAACTTTTAG
ATCTGCTGTATAAGGCGTACGGTATGCCGCCTTCGGCAAGCGCGGGCCAGAACTTGTA
A
(Seq ID No. 48)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGKVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1E217K (codonopt1)-GG-6His-GG-10His-GG:
(Seq ID No. 49)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTAAAGTCGGCTCAGCCGAAGTGGTTAAAGAGGCGGTGGATA
TTCTGAAAACCGAACGCCTCGGCCACGGTTACCACACCTTGGAAGACCAAGCACTGT
ATAACCGTCTGCGCCAGGAGAATATGCATTTTGAAATCTGCCCCTGGAGCAGTTACTT
AACTGGCGCCTGGAAACCTGACACAGAACATGCAGTGATTCGTTTGAAAAACGATCA
GGCGAATTATTCTCTTAACACGGATGATCCGCTGATATTCAAATCCACCCTGGATACCG
ACTACCAGATGACCAAACGCGATATGGGGTTTACAGAGGAAGAATTTAAACGTCTGA
ATATTAACGCGGCGAAATCAAGCTTTTTGCCAGAGGATGAAAAGCGCGAACTTTTAG
ATCTGCTGTATAAGGCGTACGGTATGCCGCCTTCGGCAAGCGCGGGCCAGAACTTGG
GCGGTCATCATCACCACCATCACGGCGGCCATCACCATCACCATCACCATCACCATCA
CGGGGGTTAA
(Seq ID No. 50)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGKVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1E217K (codonopt1)-TEV-GG-6His-GG-6His:
(Seq ID No. 51)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTAAAGTCGGCTCAGCCGAAGTGGTTAAAGAGGCGGTGGATA
TTCTGAAAACCGAACGCCTCGGCCACGGTTACCACACCTTGGAAGACCAAGCACTGT
ATAACCGTCTGCGCCAGGAGAATATGCATTTTGAAATCTGCCCCTGGAGCAGTTACTT
AACTGGCGCCTGGAAACCTGACACAGAACATGCAGTGATTCGTTTGAAAAACGATCA
GGCGAATTATTCTCTTAACACGGATGATCCGCTGATATTCAAATCCACCCTGGATACCG
ACTACCAGATGACCAAACGCGATATGGGGTTTACAGAGGAAGAATTTAAACGTCTGA
ATATTAACGCGGCGAAATCAAGCTTTTTGCCAGAGGATGAAAAGCGCGAACTTTTAG
ATCTGCTGTATAAGGCGTACGGTATGCCGCCTTCGGCAAGCGCGGGCCAGAACTTGG
AGAACCTGTACTTCCAATCCGGCGGTCATCATCACCACCATCACGGCGGCCACCACC
ACCACCACCACTGA
(Seq ID No. 52)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGKVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLENLYFQSGGHHHHHHGGHHHHHH
HsADA1G45E (codonopt1):
(Seq ID No. 53)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGAACTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 54)
MAQTPAFDKPKVELHVHLDGSIKPETILYYGRRRGIALPANTAEELLNVIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPKVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1G45E (codonopt1)-GG-6His-GG-10His-GG:
(Seq ID No. 55)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGAACTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 56)
MAQTPAFDKPKVELHVHLDGSIKPETILYYGRRRGIALPANTAEELLNVIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPKVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1G45EK164E (codonopt1):
(Seq ID No. 57)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGAACTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 58)
MAQTPAFDKPKVELHVHLDGSIKPETILYYGRRRGIALPANTAEELLNVIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPEVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1G45EK164E (codonopt1)-GG-6His-GG-10His-GG:
(Seq ID No. 59)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGAACTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 60)
MAQTPAFDKPKVELHVHLDGSIKPETILYYGRRRGIALPANTAEELLNVIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPEVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1G134N (codonopt1):
(Seq ID No. 61)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGAATC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 62)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVNQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1G134N (codonopt1)-GG-6His-GG-10His-GG:
(Seq ID No. 63)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGAATC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 64)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVNQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1G134NK164E (codonopt1):
(Seq ID No. 65)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGAATC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 66)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVNQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1G134NK164E (codonopt1)-GG-6His-GG-10His-GG:
(Seq ID No. 67)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGAATC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 68)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVNQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1I261V (codonopt1):
(Seq ID No. 69)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAGTGTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 70)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEVCPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1I261V (codonopt1)-GG-6His-GG-10His-GG:
(Seq ID No. 71)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAGTGTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 72)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEVCPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1I281V (codonopt1):
(Seq ID No. 73)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGGTCCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 74)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVVRLKN
DQANYSLNTDDPLIFKSTLDTDYQMTKRDMGFTEEEFKRLNINAAKSSFLPEDEKRELL
DLLYKAYGMPPSASAGQNL
HsADA1I281V (codonopt1)-GG-6His-GG-10His-GG:
(Seq ID No. 75)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGGTCCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 76)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVVRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164E (codonopt1):
(Seq ID No. 77)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 78)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164E (codonopt1)-GG-6His-GG-10His-GG:
(Seq ID No. 79)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 80)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EI261V (codonopt1):
(Seq ID No. 81)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAGTGTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 82)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEVCPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EI261V (codonopt1)-GG-6His-GG-10His-GG:
(Seq ID No. 83)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAGTGTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 84)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEVCPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EI281V (codonopt1):
(Seq ID No. 85)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGGTCCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 86)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVVRLKN
DQANYSLNTDDPLIFKSTLDTDYQMTKRDMGFTEEEFKRLNINAAKSSFLPEDEKRELL
DLLYKAYGMPPSASAGQNL
HsADA1K164EI281V (codonopt1)-GG-6His-GG-10His-GG:
(Seq ID No. 87)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGGTCCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 88)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVVRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EL194F (codonopt1):
(Seq ID No. 89)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 90)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EL194F (codonopt1)-GG-6His-GG-10His-GG:
(Seq ID No. 91)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 92)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EL194FQ199E (codonopt1):
(Seq ID No. 93)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGGAAGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 94)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVEAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1K164EL194FQ199E (codonopt1)-GG-6His-GG-10His-GG:
(Seq ID No. 95)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGGAAGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 96)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVEAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1K164EL194FQ199K (codonopt1):
(Seq ID No. 97)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGAAAGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 98)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVKAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EL194FQ199K (codonopt1)-GG-6His-GG-10His-GG:
(Seq ID No. 99)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGAAAGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 100)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVKAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EL194FQ202A(codonopt1):
(Seq ID No. 101)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATGCGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 102)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVQAYAEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EL194FQ202A (codonopt1)-GG-6His-GG-10His-GG:
(Seq ID No. 103)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATGCGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 104)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVQAYAEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EL194FQ202E (codonopt1):
(Seq ID No. 105)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATGAAGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 106)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVQAYEEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1K164EL194FQ202E (codonopt1)-GG-6His-GG-10His-GG:
(Seq ID No. 107)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATGAAGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 108)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLFPGHVQAYEEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1K164EL283F (codonopt1):
(Seq ID No. 109)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTTAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 110)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRFKN
DQANYSLNTDDPLIFKSTLDTDYQMTKRDMGFTEEEFKRLNINAAKSSFLPEDEKRELL
DLLYKAYGMPPSASAGQNL
HsADA1K164EL283F (codonopt1)-GG-6His-GG-10His-GG:
(Seq ID No. 111)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTTAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 112)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRFKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EP189E (codonopt1):
(Seq ID No. 113)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTGAAGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 114)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIEGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EP189E (codonopt1)-GG-6His-GG-10His-GG:
(Seq ID No. 115)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTGAAGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 116)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIEGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EQ173NL194F (codonopt1):
(Seq ID No. 117)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACA
ATCAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 118)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYNQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EQ173NL194F (codonopt1)-GG-6His-GG-10His-GG:
(Seq ID No. 119)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACA
ATCAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 120)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYNQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EQ173RL194F (codonopt1):
(Seq ID No. 121)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
GTCAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 122)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYRQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1K164EQ173RL194F (codonopt1)-GG-6His-GG-10His-
(Seq ID No. 123)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCCGGAAGTAGTAGAGTTGTGCA
AAAAGTACCGTCAGCAAACTGTCGTTGCAATCGATCTGGCGGGTGACGAAACGATT
CCGGGGAGTTCTTTATTTCCAGGACACGTGCAGGCCTATCAGGAAGCGGTGAAATCC
GGCATCCATCGTACGGTTCACGCAGGTGAGGTCGGCTCAGCCGAAGTGGTTAAAGA
GGCGGTGGATATTCTGAAAACCGAACGCCTCGGCCACGGTTACCACACCTTGGAAG
ACCAAGCACTGTATAACCGTCTGCGCCAGGAGAATATGCATTTTGAAATCTGCCCCT
GGAGCAGTTACTTAACTGGCGCCTGGAAACCTGACACAGAACATGCAGTGATTCGTT
TGAAAAACGATCAGGCGAATTATTCTCTTAACACGGATGATCCGCTGATATTCAAAT
CCACCCTGGATACCGACTACCAGATGACCAAACGCGATATGGGGTTTACAGAGGAA
GAATTTAAACGTCTGAATATTAACGCGGCGAAATCAAGCTTTTTGCCAGAGGATGAA
AAGCGCGAACTTTTAGATCTGCTGTATAAGGCGTACGGTATGCCGCCTTCGGCAAGC
GCGGGCCAGAACTTGGGCGGTCATCATCACCACCATCACGGCGGCCATCACCATCA
CCATCACCATCACCATCACGGGGGTTAA
(Seq ID No. 124)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYRQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1K164EQ174EL194F (codonopt1):
(Seq ID No. 125)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AAGAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 126)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQEQTVVAIDLAGDETIPGSSLFPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1K164EQ174EL194F (codonopt1)-GG-6His-GG-10His-
(Seq ID No. 127)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCCGGAAGTAGTAGAGTTGTGCA
AAAAGTACCAAGAGCAAACTGTCGTTGCAATCGATCTGGCGGGTGACGAAACGATT
CCGGGGAGTTCTTTATTTCCAGGACACGTGCAGGCCTATCAGGAAGCGGTGAAATCC
GGCATCCATCGTACGGTTCACGCAGGTGAGGTCGGCTCAGCCGAAGTGGTTAAAGA
GGCGGTGGATATTCTGAAAACCGAACGCCTCGGCCACGGTTACCACACCTTGGAAG
ACCAAGCACTGTATAACCGTCTGCGCCAGGAGAATATGCATTTTGAAATCTGCCCCT
GGAGCAGTTACTTAACTGGCGCCTGGAAACCTGACACAGAACATGCAGTGATTCGTT
TGAAAAACGATCAGGCGAATTATTCTCTTAACACGGATGATCCGCTGATATTCAAAT
CCACCCTGGATACCGACTACCAGATGACCAAACGCGATATGGGGTTTACAGAGGAA
GAATTTAAACGTCTGAATATTAACGCGGCGAAATCAAGCTTTTTGCCAGAGGATGAA
AAGCGCGAACTTTTAGATCTGCTGTATAAGGCGTACGGTATGCCGCCTTCGGCAAGC
GCGGGCCAGAACTTGGGCGGTCATCATCACCACCATCACGGCGGCCATCACCATCA
CCATCACCATCACCATCACGGGGGTTAA
(Seq ID No. 128)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQEQTVVAIDLAGDETIPGSSLFPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1K164ER341K (codonopt1):
(Seq ID No. 129)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGAAAGAACTTTTA
GATCTGCTGTATAAGGCGTACGGTATGCCGCCTTCGGCAAGCGCGGGCCAGAACTTGT
AA
(Seq ID No. 130)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKKELL
DLLYKAYGMPPSASAGQNL
HsADA1K164ER341K (codonopt1)-GG-6His-GG-10His-
(Seq ID No. 131)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCCGGAAGTAGTAGAGTTGTGCA
AAAAGTACCAACAGCAAACTGTCGTTGCAATCGATCTGGCGGGTGACGAAACGATT
CCGGGGAGTTCTTTACTGCCAGGACACGTGCAGGCCTATCAGGAAGCGGTGAAATC
CGGCATCCATCGTACGGTTCACGCAGGTGAGGTCGGCTCAGCCGAAGTGGTTAAAG
AGGCGGTGGATATTCTGAAAACCGAACGCCTCGGCCACGGTTACCACACCTTGGAA
GACCAAGCACTGTATAACCGTCTGCGCCAGGAGAATATGCATTTTGAAATCTGCCCC
TGGAGCAGTTACTTAACTGGCGCCTGGAAACCTGACACAGAACATGCAGTGATTCGT
TTGAAAAACGATCAGGCGAATTATTCTCTTAACACGGATGATCCGCTGATATTCAAA
TCCACCCTGGATACCGACTACCAGATGACCAAACGCGATATGGGGTTTACAGAGGA
AGAATTTAAACGTCTGAATATTAACGCGGCGAAATCAAGCTTTTTGCCAGAGGATGA
AAAGAAAGAACTTTTAGATCTGCTGTATAAGGCGTACGGTATGCCGCCTTCGGCAAG
CGCGGGCCAGAACTTGGGCGGTCATCATCACCACCATCACGGCGGCCATCACCATC
ACCATCACCATCACCATCACGGGGGTTAA
(Seq ID No. 132)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKKELL
DLLYKAYGMPPSASAGQNLGGHHHHHHGGHHHHHHHHHHGG
HsADA1L194F (codonopt1):
(Seq ID No. 133)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 134)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1L194F (codonopt1)-GG-6His-GG-10His-GG:
(Seq ID No. 135)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 136)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1L283F (codonopt1):
(Seq ID No. 137)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTTAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 138)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRFKN
DQANYSLNTDDPLIFKSTLDTDYQMTKRDMGFTEEEFKRLNINAAKSSFLPEDEKRELL
DLLYKAYGMPPSASAGQNL
HsADA1L283F (codonopt1)-GG-6His-GG-10His-GG:
(Seq ID No. 139)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTTAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 140)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRFKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N41D (codonopt1):
(Seq ID No. 141)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCGATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 142)
MAQTPAFDKPKVELHVHLDGSIKPETILYYGRRRGIALPADTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N41D (codonopt1)-GG-6His-GG-10His-GG:
(Seq ID No. 143)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCGATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 144)
MAQTPAFDKPKVELHVHLDGSIKPETILYYGRRRGIALPADTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N41DK164E (codonopt1):
(Seq ID No. 145)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCGATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 146)
MAQTPAFDKPKVELHVHLDGSIKPETILYYGRRRGIALPADTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N41DK164E (codonopt1)-GG-6His-GG-10His-GG:
(Seq ID No. 147)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCGATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 148)
MAQTPAFDKPKVELHVHLDGSIKPETILYYGRRRGIALPADTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N160DK164EL194F (codonopt1):
(Seq ID No. 149)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCGATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 150)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPDWSPEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N160DK164EL194F (codonopt1)-GG-6His-GG-10His-GG:
(Seq ID No. 151)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCGATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 152)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPDWSPEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N160S (codonopt1):
(Seq ID No. 153)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 154)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N160S (codonopt1)-GG-6His-GG-10His-GG:
(Seq ID No. 155)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 156)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N1605K164E (codonopt1):
(Seq ID No. 157)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 158)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N160SK164E (codonopt1)-GG-6His-GG-10His-GG:
(Seq ID No. 159)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 160)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N1605K164EL194F (codonopt1):
(Seq ID No. 161)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 162)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N160SK164EL194F (codonopt1)-GG-6His-GG-10His-GG:
(Seq ID No. 163)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 164)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1N1605K164EP189EL194F (codonopt1):
(Seq ID No. 165)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGGAAGTAGTAGAGTTGTGCAAAAAGTACC
AACAGCAAACTGTCGTTGCAATCGATCTGGCGGGTGACGAAACGATTGAAGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 166)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPEVVELC
KKYQQQTVVAIDLAGDETIEGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1N160SK164EP189EL194F (codonopt1)-GG-6His-GG-10His-GG:
(Seq ID No. 167)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCTCATGGAGCCCGGAAGTAGTAGAGTTGTGCAAAAAGTACC
AACAGCAAACTGTCGTTGCAATCGATCTGGCGGGTGACGAAACGATTGAAGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 168)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPSWSPEVVELC
KKYQQQTVVAIDLAGDETIEGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1P163LK164EL194F (codonopt1):
(Seq ID No. 169)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCTTGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 170)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSLEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1P163LK164EL194F (codonopt1)-GG-6His-GG-10His-
(Seq ID No. 171)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCTTGAAGTAGTAGAGTTGTGCA
AAAAGTACCAACAGCAAACTGTCGTTGCAATCGATCTGGCGGGTGACGAAACGATT
CCGGGGAGTTCTTTATTTCCAGGACACGTGCAGGCCTATCAGGAAGCGGTGAAATCC
GGCATCCATCGTACGGTTCACGCAGGTGAGGTCGGCTCAGCCGAAGTGGTTAAAGA
GGCGGTGGATATTCTGAAAACCGAACGCCTCGGCCACGGTTACCACACCTTGGAAG
ACCAAGCACTGTATAACCGTCTGCGCCAGGAGAATATGCATTTTGAAATCTGCCCCT
GGAGCAGTTACTTAACTGGCGCCTGGAAACCTGACACAGAACATGCAGTGATTCGTT
TGAAAAACGATCAGGCGAATTATTCTCTTAACACGGATGATCCGCTGATATTCAAAT
CCACCCTGGATACCGACTACCAGATGACCAAACGCGATATGGGGTTTACAGAGGAA
GAATTTAAACGTCTGAATATTAACGCGGCGAAATCAAGCTTTTTGCCAGAGGATGAA
AAGCGCGAACTTTTAGATCTGCTGTATAAGGCGTACGGTATGCCGCCTTCGGCAAGC
GCGGGCCAGAACTTGGGCGGTCATCATCACCACCATCACGGCGGCCATCACCATCA
CCATCACCATCACCATCACGGGGGTTAA
(Seq ID No. 172)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSLEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1P163SK164EL194F (codonopt1):
(Seq ID No. 173)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCAGCGAAGTAGTAGAGTTGTGCAAAAAGTACC
AACAGCAAACTGTCGTTGCAATCGATCTGGCGGGTGACGAAACGATTCCGGGGAGTT
CTTTATTTCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 174)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSSEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1P163SK164EL194F (codonopt1)-GG-6His-GG-10His-
(Seq ID No. 175)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCAGCGAAGTAGTAGAGTTGTGCA
AAAAGTACCAACAGCAAACTGTCGTTGCAATCGATCTGGCGGGTGACGAAACGATT
CCGGGGAGTTCTTTATTTCCAGGACACGTGCAGGCCTATCAGGAAGCGGTGAAATCC
GGCATCCATCGTACGGTTCACGCAGGTGAGGTCGGCTCAGCCGAAGTGGTTAAAGA
GGCGGTGGATATTCTGAAAACCGAACGCCTCGGCCACGGTTACCACACCTTGGAAG
ACCAAGCACTGTATAACCGTCTGCGCCAGGAGAATATGCATTTTGAAATCTGCCCCT
GGAGCAGTTACTTAACTGGCGCCTGGAAACCTGACACAGAACATGCAGTGATTCGTT
TGAAAAACGATCAGGCGAATTATTCTCTTAACACGGATGATCCGCTGATATTCAAAT
CCACCCTGGATACCGACTACCAGATGACCAAACGCGATATGGGGTTTACAGAGGAA
GAATTTAAACGTCTGAATATTAACGCGGCGAAATCAAGCTTTTTGCCAGAGGATGAA
AAGCGCGAACTTTTAGATCTGCTGTATAAGGCGTACGGTATGCCGCCTTCGGCAAGC
GCGGGCCAGAACTTGGGCGGTCATCATCACCACCATCACGGCGGCCATCACCATCA
CCATCACCATCACCATCACGGGGGTTAA
(Seq ID No. 176)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSSEVVELC
KKYQQQTVVAIDLAGDETIPGSSLFPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1P189E (codonopt1):
(Seq ID No. 177)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTGAAGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 178)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIEGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1P189E (codonopt1)-GG-6His-GG-10His-GG:
(Seq ID No. 179)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTGAAGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 180)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIEGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1R33K (codonopt1):
(Seq ID No. 181)
ATGGCTCAAACTCCGGCCTTCGACAAGCCGAAAGTCGAACTCCATGTACATCTTGATG
GAAGTATAAAACCGGAGACGATCCTGTATTATGGGCGCAAGCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 182)
MAQTPAFDKPKVELHVHLDGSIKPETILYYGRKRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1R33K (codonopt1)-GG-6His-GG-10His-
(Seq ID No. 183)
GG:ATGGCTCAAACTCCGGCCTTCGACAAGCCGAAAGTCGAACTCCATGTACATCTT
GATGGAAGTATAAAACCGGAGACGATCCTGTATTATGGGCGCAAGCGCGGTATTGC
TTTACCTGCCAATACCGCAGAAGGTCTGCTGAACGTTATCGGCATGGACAAACCCCT
CACCCTTCCGGATTTTCTGGCTAAGTTCGATTATTACATGCCGGCGATTGCTGGTTGT
CGTGAAGCCATCAAACGCATTGCTTATGAGTTTGTTGAAATGAAAGCGAAAGAAGG
CGTGGTCTACGTTGAAGTACGTTATTCTCCTCACCTGTTAGCTAATTCCAAGGTGGA
GCCAATCCCGTGGAACCAAGCCGAAGGAGATCTGACACCAGACGAAGTTGTCGCAC
TGGTGGGCCAGGGTCTCCAGGAGGGGGAACGCGACTTCGGCGTGAAAGCCCGTTCG
ATTCTTTGCTGTATGCGCCATCAGCCCAATTGGAGCCCGAAAGTAGTAGAGTTGTGC
AAAAAGTACCAACAGCAAACTGTCGTTGCAATCGATCTGGCGGGTGACGAAACGAT
TCCGGGGAGTTCTTTACTGCCAGGACACGTGCAGGCCTATCAGGAAGCGGTGAAAT
CCGGCATCCATCGTACGGTTCACGCAGGTGAGGTCGGCTCAGCCGAAGTGGTTAAA
GAGGCGGTGGATATTCTGAAAACCGAACGCCTCGGCCACGGTTACCACACCTTGGA
AGACCAAGCACTGTATAACCGTCTGCGCCAGGAGAATATGCATTTTGAAATCTGCCC
CTGGAGCAGTTACTTAACTGGCGCCTGGAAACCTGACACAGAACATGCAGTGATTC
GTTTGAAAAACGATCAGGCGAATTATTCTCTTAACACGGATGATCCGCTGATATTCA
AATCCACCCTGGATACCGACTACCAGATGACCAAACGCGATATGGGGTTTACAGAG
GAAGAATTTAAACGTCTGAATATTAACGCGGCGAAATCAAGCTTTTTGCCAGAGGAT
GAAAAGCGCGAACTTTTAGATCTGCTGTATAAGGCGTACGGTATGCCGCCTTCGGCA
AGCGCGGGCCAGAACTTGGGCGGTCATCATCACCACCATCACGGCGGCCATCACCA
TCACCATCACCATCACCATCACGGGGGTTAA
(Seq ID No. 184)
MAQTPAFDKPKVELHVHLDGSIKPETILYYGRKRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1R33KK164E (codonopt1):
(Seq ID No. 185)
ATGGCTCAAACTCCGGCCTTCGACAAGCCGAAAGTCGAACTCCATGTACATCTTGATG
GAAGTATAAAACCGGAGACGATCCTGTATTATGGGCGCAAGCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 186)
MAQTPAFDKPKVELHVHLDGSIKPETILYYGRKRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1R33KK164E (codonopt1)-GG-6His-GG-10His-
(Seq ID No. 187)
GG:ATGGCTCAAACTCCGGCCTTCGACAAGCCGAAAGTCGAACTCCATGTACATCTT
GATGGAAGTATAAAACCGGAGACGATCCTGTATTATGGGCGCAAGCGCGGTATTGC
TTTACCTGCCAATACCGCAGAAGGTCTGCTGAACGTTATCGGCATGGACAAACCCCT
CACCCTTCCGGATTTTCTGGCTAAGTTCGATTATTACATGCCGGCGATTGCTGGTTGT
CGTGAAGCCATCAAACGCATTGCTTATGAGTTTGTTGAAATGAAAGCGAAAGAAGG
CGTGGTCTACGTTGAAGTACGTTATTCTCCTCACCTGTTAGCTAATTCCAAGGTGGA
GCCAATCCCGTGGAACCAAGCCGAAGGAGATCTGACACCAGACGAAGTTGTCGCAC
TGGTGGGCCAGGGTCTCCAGGAGGGGGAACGCGACTTCGGCGTGAAAGCCCGTTCG
ATTCTTTGCTGTATGCGCCATCAGCCCAATTGGAGCCCGGAAGTAGTAGAGTTGTGC
AAAAAGTACCAACAGCAAACTGTCGTTGCAATCGATCTGGCGGGTGACGAAACGAT
TCCGGGGAGTTCTTTACTGCCAGGACACGTGCAGGCCTATCAGGAAGCGGTGAAAT
CCGGCATCCATCGTACGGTTCACGCAGGTGAGGTCGGCTCAGCCGAAGTGGTTAAA
GAGGCGGTGGATATTCTGAAAACCGAACGCCTCGGCCACGGTTACCACACCTTGGA
AGACCAAGCACTGTATAACCGTCTGCGCCAGGAGAATATGCATTTTGAAATCTGCCC
CTGGAGCAGTTACTTAACTGGCGCCTGGAAACCTGACACAGAACATGCAGTGATTC
GTTTGAAAAACGATCAGGCGAATTATTCTCTTAACACGGATGATCCGCTGATATTCA
AATCCACCCTGGATACCGACTACCAGATGACCAAACGCGATATGGGGTTTACAGAG
GAAGAATTTAAACGTCTGAATATTAACGCGGCGAAATCAAGCTTTTTGCCAGAGGAT
GAAAAGCGCGAACTTTTAGATCTGCTGTATAAGGCGTACGGTATGCCGCCTTCGGCA
AGCGCGGGCCAGAACTTGGGCGGTCATCATCACCACCATCACGGCGGCCATCACCA
TCACCATCACCATCACCATCACGGGGGTTAA
(Seq ID No. 188)
MAQTPAFDKPKVELHVHLDGSIKPETILYYGRKRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1R142Q (codonopt1):
(Seq ID No. 189)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACAGGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
GA
(Seq ID No. 190)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGEQDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1R142Q (codonopt1)-TEV-GG-6His-GG-
(Seq ID No. 191)
6His:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACAGGACTTCGGCGTGAAAGCCCGTTCGA
TTCTTTGCTGTATGCGCCATCAGCCCAATTGGAGCCCGAAAGTAGTAGAGTTGTGCA
AAAAGTACCAACAGCAAACTGTCGTTGCAATCGATCTGGCGGGTGACGAAACGATT
CCGGGGAGTTCTTTACTGCCAGGACACGTGCAGGCCTATCAGGAAGCGGTGAAATC
CGGCATCCATCGTACGGTTCACGCAGGTGAGGTCGGCTCAGCCGAAGTGGTTAAAG
AGGCGGTGGATATTCTGAAAACCGAACGCCTCGGCCACGGTTACCACACCTTGGAA
GACCAAGCACTGTATAACCGTCTGCGCCAGGAGAATATGCATTTTGAAATCTGCCCC
TGGAGCAGTTACTTAACTGGCGCCTGGAAACCTGACACAGAACATGCAGTGATTCGT
TTGAAAAACGATCAGGCGAATTATTCTCTTAACACGGATGATCCGCTGATATTCAAA
TCCACCCTGGATACCGACTACCAGATGACCAAACGCGATATGGGGTTTACAGAGGA
AGAATTTAAACGTCTGAATATTAACGCGGCGAAATCAAGCTTTTTGCCAGAGGATGA
AAAGCGCGAACTTTTAGATCTGCTGTATAAGGCGTACGGTATGCCGCCTTCGGCAAG
CGCGGGCCAGAACTTGGAGAACCTGTACTTCCAATCCGGCGGTCATCATCACCACCA
TCACGGCGGCCACCACCACCACCACCACTGA
(Seq ID No. 192)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGEQDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLENLYFQSGGHHHHHHGGHHHHHH
HsADA1R341K (codonopt1):
(Seq ID No. 193)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGAAAGAACTTTTA
GATCTGCTGTATAAGGCGTACGGTATGCCGCCTTCGGCAAGCGCGGGCCAGAACTTGT
AA
(Seq ID No. 194)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKKELL
DLLYKAYGMPPSASAGQNL
HsADA1R341K (codonopt1)-GG-6His-GG-10His-GG:
(Seq ID No. 195)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGAAAGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 196)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKKELL
DLLYKAYGMPPSASAGQNLGGHHHHHHGGHHHHHHHHHHGG
HsADA1S21A (codonopt1):
(Seq ID No. 197)
ATGGCTCAAACTCCGGCCTTCGACAAGCCGAAAGTCGAACTCCATGTACATCTTGATG
GAGCCATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 198)
MAQTPAFDKPKVELHVHLDGAIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1S21A (codonopt1)-GG-6His-GG-10His-GG:
(Seq ID No. 199)
ATGGCTCAAACTCCGGCCTTCGACAAGCCGAAAGTCGAACTCCATGTACATCTTGATG
GAGCCATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 200)
MAQTPAFDKPKVELHVHLDGAIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1S21AK164E (codonopt1):
(Seq ID No. 201)
ATGGCTCAAACTCCGGCCTTCGACAAGCCGAAAGTCGAACTCCATGTACATCTTGATG
GAGCCATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 202)
MAQTPAFDKPKVELHVHLDGAIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1S21AK164E (codonopt1)-GG-6His-GG-10His-GG:
(Seq ID No. 203)
ATGGCTCAAACTCCGGCCTTCGACAAGCCGAAAGTCGAACTCCATGTACATCTTGATG
GAGCCATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 204)
MAQTPAFDKPKVELHVHLDGAIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1T57S (codonopt1):
(Seq ID No. 205)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCTCCCTT
CCGGATTTTCTGGCTAAGTTCGATTATTACATGCCGGCGATTGCTGGTTGTCGTGAAGC
CATCAAACGCATTGCTTATGAGTTTGTTGAAATGAAAGCGAAAGAAGGCGTGGTCTA
CGTTGAAGTACGTTATTCTCCTCACCTGTTAGCTAATTCCAAGGTGGAGCCAATCCCG
TGGAACCAAGCCGAAGGAGATCTGACACCAGACGAAGTTGTCGCACTGGTGGGCCA
GGGTCTCCAGGAGGGGGAACGCGACTTCGGCGTGAAAGCCCGTTCGATTCTTTGCTG
TATGCGCCATCAGCCCAATTGGAGCCCGAAAGTAGTAGAGTTGTGCAAAAAGTACCA
ACAGCAAACTGTCGTTGCAATCGATCTGGCGGGTGACGAAACGATTCCGGGGAGTTC
TTTACTGCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 206)
MAQTPAFDKPKVELHVHLDGSIKPETILYYGRRRGIALPANTAEGLLNVIGM
DKPLSLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1T57S (codonopt1)-GG-6His-GG-10His-GG:
(Seq ID No. 207)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCTCCCTT
CCGGATTTTCTGGCTAAGTTCGATTATTACATGCCGGCGATTGCTGGTTGTCGTGAAGC
CATCAAACGCATTGCTTATGAGTTTGTTGAAATGAAAGCGAAAGAAGGCGTGGTCTA
CGTTGAAGTACGTTATTCTCCTCACCTGTTAGCTAATTCCAAGGTGGAGCCAATCCCG
TGGAACCAAGCCGAAGGAGATCTGACACCAGACGAAGTTGTCGCACTGGTGGGCCA
GGGTCTCCAGGAGGGGGAACGCGACTTCGGCGTGAAAGCCCGTTCGATTCTTTGCTG
TATGCGCCATCAGCCCAATTGGAGCCCGAAAGTAGTAGAGTTGTGCAAAAAGTACCA
ACAGCAAACTGTCGTTGCAATCGATCTGGCGGGTGACGAAACGATTCCGGGGAGTTC
TTTACTGCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 208)
MAQTPAFDKPKVELHVHLDGSIKPETILYYGRRRGIALPANTAEGLLNVIGM
DKPLSLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1T57SK164E (codonopt1):
(Seq ID No. 209)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCTCCCTT
CCGGATTTTCTGGCTAAGTTCGATTATTACATGCCGGCGATTGCTGGTTGTCGTGAAGC
CATCAAACGCATTGCTTATGAGTTTGTTGAAATGAAAGCGAAAGAAGGCGTGGTCTA
CGTTGAAGTACGTTATTCTCCTCACCTGTTAGCTAATTCCAAGGTGGAGCCAATCCCG
TGGAACCAAGCCGAAGGAGATCTGACACCAGACGAAGTTGTCGCACTGGTGGGCCA
GGGTCTCCAGGAGGGGGAACGCGACTTCGGCGTGAAAGCCCGTTCGATTCTTTGCTG
TATGCGCCATCAGCCCAATTGGAGCCCGGAAGTAGTAGAGTIGTGCAAAAAGTACCA
ACAGCAAACTGTCGTTGCAATCGATCTGGCGGGTGACGAAACGATTCCGGGGAGTTC
TTTACTGCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGTAA
(Seq ID No. 210)
MAQTPAFDKPKVELHVHLDGSIKPETILYYGRRRGIALPANTAEGLLNVIGM
DKPLSLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNL
HsADA1T57SK164E (codonopt1)-GG-6His-GG-10His-GG:
(Seq ID No. 211)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCTCCCTT
CCGGATTTTCTGGCTAAGTTCGATTATTACATGCCGGCGATTGCTGGTTGTCGTGAAGC
CATCAAACGCATTGCTTATGAGTTTGTTGAAATGAAAGCGAAAGAAGGCGTGGTCTA
CGTTGAAGTACGTTATTCTCCTCACCTGTTAGCTAATTCCAAGGTGGAGCCAATCCCG
TGGAACCAAGCCGAAGGAGATCTGACACCAGACGAAGTTGTCGCACTGGTGGGCCA
GGGTCTCCAGGAGGGGGAACGCGACTTCGGCGTGAAAGCCCGTTCGATTCTTTGCTG
TATGCGCCATCAGCCCAATTGGAGCCCGGAAGTAGTAGAGTTGTGCAAAAAGTACCA
ACAGCAAACTGTCGTTGCAATCGATCTGGCGGGTGACGAAACGATTCCGGGGAGTTC
TTTACTGCCAGGACACGTGCAGGCCTATCAGGAAGCGGTGAAATCCGGCATCCATCG
TACGGTTCACGCAGGTGAGGTCGGCTCAGCCGAAGTGGTTAAAGAGGCGGTGGATAT
TCTGAAAACCGAACGCCTCGGCCACGGTTACCACACCTTGGAAGACCAAGCACTGTA
TAACCGTCTGCGCCAGGAGAATATGCATTTTGAAATCTGCCCCTGGAGCAGTTACTTA
ACTGGCGCCTGGAAACCTGACACAGAACATGCAGTGATTCGTTTGAAAAACGATCAG
GCGAATTATTCTCTTAACACGGATGATCCGCTGATATTCAAATCCACCCTGGATACCGA
CTACCAGATGACCAAACGCGATATGGGGTTTACAGAGGAAGAATTTAAACGTCTGAA
TATTAACGCGGCGAAATCAAGCTTTTTGCCAGAGGATGAAAAGCGCGAACTTTTAGAT
CTGCTGTATAAGGCGTACGGTATGCCGCCTTCGGCAAGCGCGGGCCAGAACTTGGGC
GGTCATCATCACCACCATCACGGCGGCCATCACCATCACCATCACCATCACCATCACG
GGGGTTAA
(Seq ID No. 212)
MAQTPAFDKPKVELHVHLDGSIKPETILYYGRRRGIALPANTAEGLLNVIGM
DKPLSLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPEVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGGHHHHHHHHHHGG
HsADA1V49I (codonopt1):
(Seq ID No. 213)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACATCATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 214)
MAQTPAFDKPKVELHVHLDGSIKPETILYYGRRRGIALPANTAEGLLNIIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPKVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1V49I (codonopt1)-GG-6His-GG-10His-GG:
(Seq ID No. 215)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACATCATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTG
GGCGGTCATCATCACCACCATCACGGCGGCCATCACCATCACCATCACCATCACCATC
ACGGGGGTTAA
(Seq ID No. 216)
MAQTPAFDKPKVELHVHLDGSIKPETILYYGRRRGIALPANTAEGLLNIIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPKVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
HsADA1V49IK164E (codonopt1):
(Seq ID No. 217)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACATCATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGGAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGCCGCCTTCGGCAAGCGCGGGCCAGAACTTGT
AA
(Seq ID No. 218)
MAQTPAFDKPKVELHVHLDGSIKPETILYYGRRRGIALPANTAEGLLNIIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPEVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNL
HsADA1V49IK164E (codonopt1)-GG-6His-GG-10His-
(Seq ID No. 219)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACATCATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCCGGAAGTAGTAGAGTTGTGCA
AAAAGTACCAACAGCAAACTGTCGTTGCAATCGATCTGGCGGGTGACGAAACGATT
CCGGGGAGTTCTTTACTGCCAGGACACGTGCAGGCCTATCAGGAAGCGGTGAAATC
CGGCATCCATCGTACGGTTCACGCAGGTGAGGTCGGCTCAGCCGAAGTGGTTAAAG
AGGCGGTGGATATTCTGAAAACCGAACGCCTCGGCCACGGTTACCACACCTTGGAA
GACCAAGCACTGTATAACCGTCTGCGCCAGGAGAATATGCATTTTGAAATCTGCCCC
TGGAGCAGTTACTTAACTGGCGCCTGGAAACCTGACACAGAACATGCAGTGATTCGT
TTGAAAAACGATCAGGCGAATTATTCTCTTAACACGGATGATCCGCTGATATTCAAA
TCCACCCTGGATACCGACTACCAGATGACCAAACGCGATATGGGGTTTACAGAGGA
AGAATTTAAACGTCTGAATATTAACGCGGCGAAATCAAGCTTTTTGCCAGAGGATGA
AAAGCGCGAACTTTTAGATCTGCTGTATAAGGCGTACGGTATGCCGCCTTCGGCAAG
CGCGGGCCAGAACTTGGGCGGTCATCATCACCACCATCACGGCGGCCATCACCATC
ACCATCACCATCACCATCACGGGGGTTAA
(Seq ID No. 220)
MAQTPAFDKPKVELHVHLDGSIKPETILYYGRRRGIALPANTAEGLLNIIGMD
KPLTLPDFLAKFDYYMPAIAGCREAIKRIAYEFVEMKAKEGVVYVEVRYSPHLLANSKV
EPIPWNQAEGDLTPDEVVALVGQGLQEGERDFGVKARSILCCMRHQPNWSPEVVELCK
KYQQQTVVAIDLAGDETIPGSSLLPGHVQAYQEAVKSGIHRTVHAGEVGSAEVVKEAV
DILKTERLGHGYHTLEDQALYNRLRQENMHFEICPWSSYLTGAWKPDTEHAVIRLKND
QANYSLNTDDPLIFKSTLDTDYQMTKRDMGFTEEEFKRLNINAAKSSFLPEDEKRELLDL
LYKAYGMPPSASAGQNLGGHHHHHHGGHHHHHHHHHHGG
OtherconstructsrelatedtootherattemptsatHsADA1purifications:
HsADA1“missingtail” (codonopt1):
(Seq ID No. 221)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGTAA
(Seq ID No. 222)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGM
HsADA1“missingtail” (codonopt1)-GG-6His-GG-10His-
(Seq ID No. 223)
GG:ATGGCTCAAACTCCGGCCTTCGACAAGCCGAAAGTCGAACTCCATGTACATCTT
GATGGAAGTATAAAACCGGAGACGATCCTGTATTATGGGCGCCGTCGCGGTATTGCT
TTACCTGCCAATACCGCAGAAGGTCTGCTGAACGTTATCGGCATGGACAAACCCCTC
ACCCTTCCGGATTTTCTGGCTAAGTTCGATTATTACATGCCGGCGATTGCTGGTTGTC
GTGAAGCCATCAAACGCATTGCTTATGAGTTTGTTGAAATGAAAGCGAAAGAAGGC
GTGGTCTACGTTGAAGTACGTTATTCTCCTCACCTGTTAGCTAATTCCAAGGTGGAG
CCAATCCCGTGGAACCAAGCCGAAGGAGATCTGACACCAGACGAAGTTGTCGCACT
GGTGGGCCAGGGTCTCCAGGAGGGGGAACGCGACTTCGGCGTGAAAGCCCGTTCGA
TTCTTTGCTGTATGCGCCATCAGCCCAATTGGAGCCCGAAAGTAGTAGAGTTGTGCA
AAAAGTACCAACAGCAAACTGTCGTTGCAATCGATCTGGCGGGTGACGAAACGATT
CCGGGGAGTTCTTTACTGCCAGGACACGTGCAGGCCTATCAGGAAGCGGTGAAATC
CGGCATCCATCGTACGGTTCACGCAGGTGAGGTCGGCTCAGCCGAAGTGGTTAAAG
AGGCGGTGGATATTCTGAAAACCGAACGCCTCGGCCACGGTTACCACACCTTGGAA
GACCAAGCACTGTATAACCGTCTGCGCCAGGAGAATATGCATTTTGAAATCTGCCCC
TGGAGCAGTTACTTAACTGGCGCCTGGAAACCTGACACAGAACATGCAGTGATTCGT
TTGAAAAACGATCAGGCGAATTATTCTCTTAACACGGATGATCCGCTGATATTCAAA
TCCACCCTGGATACCGACTACCAGATGACCAAACGCGATATGGGGTTTACAGAGGA
AGAATTTAAACGTCTGAATATTAACGCGGCGAAATCAAGCTTTTTGCCAGAGGATGA
AAAGCGCGAACTTTTAGATCTGCTGTATAAGGCGTACGGTATGGGCGGTCATCATCA
CCACCATCACGGCGGCCATCACCATCACCATCACCATCACCATCACGGGGGTTAA
(Seq ID No. 224)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMGGHHHHHHGGHHHHHHHHHHGG
HsADA1“missingtail” (codonopt1)-TEV-GG-6His-GG-6His:
(Seq ID No. 225)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGGAGAACCTGTACTTCCAATCCGGCGGTCATC
ATCACCACCATCACGGCGGCCACCACCACCACCACCACTGA
(Seq ID No. 226)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMENLYFQSGGHHHHHHGGHHHHHH
HsADA1“missingtail” (codonopt1)-TEV-GG-6His-GG-10His-GG:
(Seq ID No. 227)
ATGGCTCAAACTCCGGCCTTCGACAAGCCGAAAGTCGAACTCCATGTACATCTTGATG
GAAGTATAAAACCGGAGACGATCCTGTATTATGGGCGCCGTCGCGGTATTGCTTTACC
TGCCAATACCGCAGAAGGTCTGCTGAACGTTATCGGCATGGACAAACCCCTCACCCT
TCCGGATTTTCTGGCTAAGTTCGATTATTACATGCCGGCGATTGCTGGTTGTCGTGAAG
CCATCAAACGCATTGCTTATGAGTTTGTTGAAATGAAAGCGAAAGAAGGCGTGGTCT
ACGTTGAAGTACGTTATTCTCCTCACCTGTTAGCTAATTCCAAGGTGGAGCCAATCCC
GTGGAACCAAGCCGAAGGAGATCTGACACCAGACGAAGTTGTCGCACTGGTGGGCC
AGGGTCTCCAGGAGGGGGAACGCGACTTCGGCGTGAAAGCCCGTTCGATTCTTTGCT
GTATGCGCCATCAGCCCAATTGGAGCCCGAAAGTAGTAGAGTTGTGCAAAAAGTACC
AACAGCAAACTGTCGTTGCAATCGATCTGGCGGGTGACGAAACGATTCCGGGGAGTT
CTTTACTGCCAGGACACGTGCAGGCCTATCAGGAAGCGGTGAAATCCGGCATCCATC
GTACGGTTCACGCAGGTGAGGTCGGCTCAGCCGAAGTGGTTAAAGAGGCGGTGGAT
ATTCTGAAAACCGAACGCCTCGGCCACGGTTACCACACCTTGGAAGACCAAGCACTG
TATAACCGTCTGCGCCAGGAGAATATGCATTTTGAAATCTGCCCCTGGAGCAGTTACT
TAACTGGCGCCTGGAAACCTGACACAGAACATGCAGTGATTCGTTTGAAAAACGATC
AGGCGAATTATTCTCTTAACACGGATGATCCGCTGATATTCAAATCCACCCTGGATACC
GACTACCAGATGACCAAACGCGATATGGGGTTTACAGAGGAAGAATTTAAACGTCTG
AATATTAACGCGGCGAAATCAAGCTTTTTGCCAGAGGATGAAAAGCGCGAACTTTTA
GATCTGCTGTATAAGGCGTACGGTATGGAGAACCTGTACTTCCAATCCGGCGGTCATC
ATCACCACCATCACGGCGGCCATCACCATCACCATCACCATCACCATCACGGGGGTTA
A
(Seq ID No. 228)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMENLYFQSGGHHHHHHGGHHHHHHHHHHGG
N-6His-Thrombin-MBP-Thrombin-TEV-HsADA1 (codonopt1)-GG-6His-GG:
(Seq ID No. 229)
ATGGGCAGCAGCCATCATCATCATCATCACAGCAGCGGCCTGGTGCCGCGCGGCAGC
CATATGAAAATCGAAGAAGGTAAACTGGTAATCTGGATTAACGGCGATAAAGGCTATA
ACGGTCTCGCTGAAGTCGGTAAGAAATTCGAGAAAGATACCGGAATTAAAGTCACCG
TTGAGCATCCGGATAAACTGGAAGAGAAATTCCCACAGGTTGCGGCAACTGGCGATG
GCCCTGACATTATCTTCTGGGCACACGACCGCTTTGGTGGCTACGCTCAATCTGGCCT
GTTGGCTGAAATCACCCCGGACAAAGCGTTCCAGGACAAGCTGTATCCGTTTACCTG
GGATGCCGTACGTTACAACGGCAAGCTGATTGCTTACCCGATCGCTGTTGAAGCGTTA
TCGCTGATTTATAACAAAGATCTGCTGCCGAACCCGCCAAAAACCTGGGAAGAGATC
CCGGCGCTGGATAAAGAACTGAAAGCGAAAGGTAAGAGCGCGCTGATGTTCAACCT
GCAAGAACCGTACTTCACCTGGCCGCTGATTGCTGCTGACGGGGGTTATGCGTTCAA
GTATGAAAACGGCAAGTACGACATTAAAGACGTGGGCGTGGATAACGCTGGCGCGAA
AGCGGGTCTGACCTTCCTGGTTGACCTGATTAAAAACAAACACATGAATGCAGACAC
CGATTACTCCATCGCAGAAGCTGCCTTTAATAAAGGCGAAACAGCGATGACCATCAAC
GGCCCGTGGGCATGGTCCAACATCGACACCAGCAAAGTGAATTATGGTGTAACGGTA
CTGCCGACCTTCAAGGGTCAACCATCCAAACCGTTCGTTGGCGTGCTGAGCGCAGGT
ATTAACGCCGCCAGTCCGAACAAAGAGCTGGCGAAAGAGTTCCTCGAAAACTATCTG
CTGACTGATGAAGGTCTGGAAGCGGTTAATAAAGACAAACCGCTGGGTGCCGTAGCG
CTGAAGTCTTACGAGGAAGAGTTGGCGAAAGATCCACGTATTGCCGCCACCATGGAA
AACGCCCAGAAAGGTGAAATCATGCCGAACATCCCGCAGATGTCCGCTTTCTGGTAT
GCCGTGCGTACTGCGGTGATCAACGCCGCCAGCGGTCGTCAGACTGTCGATGAAGCC
CTGAAAGACGCGCAGACTAATTCGAGCTCCCACCATCACCATCACCACGCGAATTCG
GTACCGCTGGTTCCGCGTGGATCTGAGAACCTGTACTTCCAATCCATGGCTCAAACTC
CGGCCTTCGACAAGCCGAAAGTCGAACTCCATGTACATCTTGATGGAAGTATAAAAC
CGGAGACGATCCTGTATTATGGGCGCCGTCGCGGTATTGCTTTACCTGCCAATACCGC
AGAAGGTCTGCTGAACGTTATCGGCATGGACAAACCCCTCACCCTTCCGGATTTTCTG
GCTAAGTTCGATTATTACATGCCGGCGATTGCTGGTTGTCGTGAAGCCATCAAACGCA
TTGCTTATGAGTTTGTTGAAATGAAAGCGAAAGAAGGCGTGGTCTACGTTGAAGTAC
GTTATTCTCCTCACCTGTTAGCTAATTCCAAGGTGGAGCCAATCCCGTGGAACCAAGC
CGAAGGAGATCTGACACCAGACGAAGTTGTCGCACTGGTGGGCCAGGGTCTCCAGG
AGGGGGAACGCGACTTCGGCGTGAAAGCCCGTTCGATTCTTTGCTGTATGCGCCATC
AGCCCAATTGGAGCCCGAAAGTAGTAGAGTTGTGCAAAAAGTACCAACAGCAAACT
GTCGTTGCAATCGATCTGGCGGGTGACGAAACGATTCCGGGGAGTTCTTTACTGCCA
GGACACGTGCAGGCCTATCAGGAAGCGGTGAAATCCGGCATCCATCGTACGGTTCAC
GCAGGTGAGGTCGGCTCAGCCGAAGTGGTTAAAGAGGCGGTGGATATTCTGAAAAC
CGAACGCCTCGGCCACGGTTACCACACCTTGGAAGACCAAGCACTGTATAACCGTCT
GCGCCAGGAGAATATGCATTTTGAAATCTGCCCCTGGAGCAGTTACTTAACTGGCGCC
TGGAAACCTGACACAGAACATGCAGTGATTCGTTTGAAAAACGATCAGGCGAATTAT
TCTCTTAACACGGATGATCCGCTGATATTCAAATCCACCCTGGATACCGACTACCAGAT
GACCAAACGCGATATGGGGTTTACAGAGGAAGAATTTAAACGTCTGAATATTAACGCG
GCGAAATCAAGCTTTTTGCCAGAGGATGAAAAGCGCGAACTTTTAGATCTGCTGTATA
AGGCGTACGGTATGCCGCCTTCGGCAAGCGCGGGCCAGAACTTGGGCGGTCATCATC
ACCACCATCACGGCGGCTAA
(Seq ID No. 230)
MGSSHHHHHHSSGLVPRGSHMKIEEGKLVIWINGDKGYNGLAEVGKKFEKD
TGIKVTVEHPDKLEEKFPQVAATGDGPDIIFWAHDRFGGYAQSGLLAEITPDKAFQDKL
YPFTWDAVRYNGKLIAYPIAVEALSLIYNKDLLPNPPKTWEEIPALDKELKAKGKSALM
FNLQEPYFTWPLIAADGGYAFKYENGKYDIKDVGVDNAGAKAGLTFLVDLIKNKHMN
ADTDYSIAEAAFNKGETAMTINGPWAWSNIDTSKVNYGVTVLPTFKGQPSKPFVGVLSA
GINAASPNKELAKEFLENYLLTDEGLEAVNKDKPLGAVALKSYEEELAKDPRIAATMEN
AQKGEIMPNIPQMSAFWYAVRTAVINAASGRQTVDEALKDAQTNSSSHHHHHHANSVP
LVPRGSENLYFQSMAQTPAFDKPKVELHVHLDGSIKPETILYYGRRRGIALPANTAEGLL
NVIGMDKPLTLPDFLAKFDYYMPAIAGCREAIKRIAYEFVEMKAKEGVVYVEVRYSPHL
LANSKVEPIPWNQAEGDLTPDEVVALVGQGLQEGERDFGVKARSILCCMRHQPNWSPK
VVELCKKYQQQTVVAIDLAGDETIPGSSLLPGHVQAYQEAVKSGIHRTVHAGEVGSAEV
VKEAVDILKTERLGHGYHTLEDQALYNRLRQENMHFEICPWSSYLTGAWKPDTEHAVI
RLKNDQANYSLNTDDPLIFKSTLDTDYQMTKRDMGFTEEEFKRLNINAAKSSFLPEDEK
RELLDLLYKAYGMPPSASAGQNLGGHHHHHHGG
Constructsforhuman(Hs),cow(Bt), andmouse(Mm)ADAlswithN′terminalHisTaginstead
ofmoreroutinelyutilizedC′TerminalHisTag
N-GG-6His-GG-BtADA:
(Seq ID No. 231)
ATGGGCGGTCATCATCACCACCATCACGGCGGCGCTCAAACTCCCGCCTTCAATAAGC
CTAAAGTCGAGCTCCACGTTCATCTGGACGGGGCGATAAAACCAGAAACAATCCTGT
ACTACGGTCGCAAACGTGGGATTGCCCTGCCGGCAGATACGCCGGAAGAGCTGCAG
AACATCATTGGCATGGATAAGCCGCTGAGTCTTCCTGAATTTTTGGCTAAATTCGACTA
CTATATGCCCGCCATCGCAGGTTGTCGCGAAGCGGTGAAACGTATTGCGTACGAGTTT
GTAGAAATGAAAGCTAAGGATGGCGTGGTCTATGTTGAAGTGCGCTACTCACCACAC
CTCTTAGCAAATAGCAAAGTAGAGCCGATCCCGTGGAACCAGGCCGAAGGCGACCTG
ACCCCGGATGAAGTCGTGTCCCTGGTTAATCAAGGTCTGCAGGAGGGCGAACGTGAT
TTCGGCGTGAAAGTACGCAGTATATTATGCTGTATGCGTCATCAGCCTTCGTGGAGCTC
CGAAGTCGTTGAGCTTTGCAAAAAGTATCGCGAACAAACCGTTGTGGCGATCGACCT
CGCAGGTGATGAAACTATTGAGGGCAGTTCTTTGTTTCCAGGCCACGTCAAAGCTTA
CGCGGAAGCGGTTAAATCAGGAGTGCATCGTACAGTGCACGCCGGTGAAGTGGGCTC
GGCAAACGTCGTAAAAGAGGCGGTTGATACGTTAAAGACCGAACGCCTGGGACATG
GTTATCACACCCTGGAAGACGCAACGCTGTACAATCGTCTGCGCCAGGAGAACATGC
ATTTCGAAGTGTGTCCCTGGAGCTCGTATCTCACTGGAGCCTGGAAACCGGATACAG
AACATCCGGTGGTTCGTTTTAAAAATGACCAGGTAAACTATTCTCTTAATACGGATGAT
CCGTTGATTTTCAAATCAACCTTAGACACCGATTATCAAATGACGAAGAACGAGATGG
GGTTTACCGAAGAAGAGTTTAAACGCCTGAATATTAACGCGGCGAAAAGCAGCTTTC
TGCCTGAAGATGAAAAAAAGGAATTACTGGATCTGCTTTATAAAGCGTATGGTATGCC
ATCTCCGGCCTCCGCTGAACAGTGCTTGTAA
(Seq ID No. 232)
MGGHHHHHHGGAQTPAFNKPKVELHVHLDGAIKPETILYYGRKRGIALPAD
TPEELQNIIGMDKPLSLPEFLAKFDYYMPAIAGCREAVKRIAYEFVEMKAKDGVVYVEV
RYSPHLLANSKVEPIPWNQAEGDLTPDEVVSLVNQGLQEGERDFGVKVRSILCCMRHQP
SWSSEVVELCKKYREQTVVAIDLAGDETIEGSSLFPGHVKAYAEAVKSGVHRTVHAGE
VGSANVVKEAVDTLKTERLGHGYHTLEDATLYNRLRQENMHFEVCPWSSYLTGAWKP
DTEHPVVRFKNDQVNYSLNTDDPLIFKSTLDTDYQMTKNEMGFTEEEFKRLNINAAKSS
FLPEDEKKELLDLLYKAYGMPSPASAEQCL
N-GG-6His-GG-BtADAC57S:
(Seq ID No. 233)
ATGGGCGGTCATCATCACCACCATCACGGCGGCGCTCAAACTCCCGCCTTCAATAAGC
CTAAAGTCGAGCTCCACGTTCATCTGGACGGGGCGATAAAACCAGAAACAATCCTGT
ACTACGGTCGCAAACGTGGGATTGCCCTGCCGGCAGATACGCCGGAAGAGCTGCAG
AACATCATTGGCATGGATAAGCCGCTGAGTCTTCCTGAATTTTTGGCTAAATTCGACTA
CTATATGCCCGCCATCGCAGGTTCTCGCGAAGCGGTGAAACGTATTGCGTACGAGTTT
GTAGAAATGAAAGCTAAGGATGGCGTGGTCTATGTTGAAGTGCGCTACTCACCACAC
CTCTTAGCAAATAGCAAAGTAGAGCCGATCCCGTGGAACCAGGCCGAAGGCGACCTG
ACCCCGGATGAAGTCGTGTCCCTGGTTAATCAAGGTCTGCAGGAGGGCGAACGTGAT
TTCGGCGTGAAAGTACGCAGTATATTATGCTGTATGCGTCATCAGCCTTCGTGGAGCTC
CGAAGTCGTTGAGCTTTGCAAAAAGTATCGCGAACAAACCGTTGTGGCGATCGACCT
CGCAGGTGATGAAACTATTGAGGGCAGTTCTTTGTTTCCAGGCCACGTCAAAGCTTA
CGCGGAAGCGGTTAAATCAGGAGTGCATCGTACAGTGCACGCCGGTGAAGTGGGCTC
GGCAAACGTCGTAAAAGAGGCGGTTGATACGTTAAAGACCGAACGCCTGGGACATG
GTTATCACACCCTGGAAGACGCAACGCTGTACAATCGTCTGCGCCAGGAGAACATGC
ATTTCGAAGTGTGTCCCTGGAGCTCGTATCTCACTGGAGCCTGGAAACCGGATACAG
AACATCCGGTGGTTCGTTTTAAAAATGACCAGGTAAACTATTCTCTTAATACGGATGAT
CCGTTGATTTTCAAATCAACCTTAGACACCGATTATCAAATGACGAAGAACGAGATGG
GGTTTACCGAAGAAGAGTTTAAACGCCTGAATATTAACGCGGCGAAAAGCAGCTTTC
TGCCTGAAGATGAAAAAAAGGAATTACTGGATCTGCTTTATAAAGCGTATGGTATGCC
ATCTCCGGCCTCCGCTGAACAGTGCTTGTAA
(Seq ID No. 234)
MGGHHHHHHGGAQTPAFNKPKVELHVHLDGAIKPETILYYGRKRGIALPAD
TPEELQNIIGMDKPLSLPEFLAKFDYYMPAIAGSREAVKRIAYEFVEMKAKDGVVYVEV
RYSPHLLANSKVEPIPWNQAEGDLTPDEVVSLVNQGLQEGERDFGVKVRSILCCMRHQP
SWSSEVVELCKKYREQTVVAIDLAGDETIEGSSLFPGHVKAYAEAVKSGVHRTVHAGE
VGSANVVKEAVDTLKTERLGHGYHTLEDATLYNRLRQENMHFEVCPWSSYLTGAWKP
DTEHPVVRFKNDQVNYSLNTDDPLIFKSTLDTDYQMTKNEMGFTEEEFKRLNINAAKSS
FLPEDEKKELLDLLYKAYGMPSPASAEQCL
N-GG-6His-GG-HsADA1:
(Seq ID No. 235)
ATGGGCGGTCATCATCACCACCATCACGGCGGCGCTCAAACTCCGGCCTTCGACAAG
CCGAAAGTCGAACTCCATGTACATCTTGATGGAAGTATAAAACCGGAGACGATCCTGT
ATTATGGGCGCCGTCGCGGTATTGCTTTACCTGCCAATACCGCAGAAGGTCTGCTGAA
CGTTATCGGCATGGACAAACCCCTCACCCTTCCGGATTTTCTGGCTAAGTTCGATTATT
ACATGCCGGCGATTGCTGGTTGTCGTGAAGCCATCAAACGCATTGCTTATGAGTTTGT
TGAAATGAAAGCGAAAGAAGGCGTGGTCTACGTTGAAGTACGTTATTCTCCTCACCT
GTTAGCTAATTCCAAGGTGGAGCCAATCCCGTGGAACCAAGCCGAAGGAGATCTGAC
ACCAGACGAAGTTGTCGCACTGGTGGGCCAGGGTCTCCAGGAGGGGGAACGCGACT
TCGGCGTGAAAGCCCGTTCGATTCTTTGCTGTATGCGCCATCAGCCCAATTGGAGCCC
GAAAGTAGTAGAGTTGTGCAAAAAGTACCAACAGCAAACTGTCGTTGCAATCGATCT
GGCGGGTGACGAAACGATTCCGGGGAGTTCTTTACTGCCAGGACACGTGCAGGCCTA
TCAGGAAGCGGTGAAATCCGGCATCCATCGTACGGTTCACGCAGGTGAGGTCGGCTC
AGCCGAAGTGGTTAAAGAGGCGGTGGATATTCTGAAAACCGAACGCCTCGGCCACG
GTTACCACACCTTGGAAGACCAAGCACTGTATAACCGTCTGCGCCAGGAGAATATGC
ATTTTGAAATCTGCCCCTGGAGCAGTTACTTAACTGGCGCCTGGAAACCTGACACAG
AACATGCAGTGATTCGTTTGAAAAACGATCAGGCGAATTATTCTCTTAACACGGATGA
TCCGCTGATATTCAAATCCACCCTGGATACCGACTACCAGATGACCAAACGCGATATG
GGGTTTACAGAGGAAGAATTTAAACGTCTGAATATTAACGCGGCGAAATCAAGCTTTT
TGCCAGAGGATGAAAAGCGCGAACTTTTAGATCTGCTGTATAAGGCGTACGGTATGCC
GCCTTCGGCAAGCGCGGGCCAGAACTTGTAA
(Seq ID No. 236)
MGGHHHHHHGGAQTPAFDKPKVELHVHLDGSIKPETILYYGRRRGIALPANT
AEGLLNVIGMDKPLTLPDFLAKFDYYMPAIAGCREAIKRIAYEFVEMKAKEGVVYVEVR
YSPHLLANSKVEPIPWNQAEGDLTPDEVVALVGQGLQEGERDFGVKARSILCCMRHQP
NWSPKVVELCKKYQQQTVVAIDLAGDETIPGSSLLPGHVQAYQEAVKSGIHRTVHAGE
VGSAEVVKEAVDILKTERLGHGYHTLEDQALYNRLRQENMHFEICPWSSYLTGAWKPD
TEHAVIRLKNDQANYSLNTDDPLIFKSTLDTDYQMTKRDMGFTEEEFKRLNINAAKSSF
LPEDEKRELLDLLYKAYGMPPSASAGQNL
N-GG-6His-GG-HsADA1C57S:
(Seq ID No. 237)
ATGGGCGGTCATCATCACCACCATCACGGCGGCGCTCAAACTCCGGCCTTCGACAAG
CCGAAAGTCGAACTCCATGTACATCTTGATGGAAGTATAAAACCGGAGACGATCCTGT
ATTATGGGCGCCGTCGCGGTATTGCTTTACCTGCCAATACCGCAGAAGGTCTGCTGAA
CGTTATCGGCATGGACAAACCCCTCACCCTTCCGGATTTTCTGGCTAAGTTCGATTATT
ACATGCCGGCGATTGCTGGTTCTCGTGAAGCCATCAAACGCATTGCTTATGAGTTTGT
TGAAATGAAAGCGAAAGAAGGCGTGGTCTACGTTGAAGTACGTTATTCTCCTCACCT
GTTAGCTAATTCCAAGGTGGAGCCAATCCCGTGGAACCAAGCCGAAGGAGATCTGAC
ACCAGACGAAGTTGTCGCACTGGTGGGCCAGGGTCTCCAGGAGGGGGAACGCGACT
TCGGCGTGAAAGCCCGTTCGATTCTTTGCTGTATGCGCCATCAGCCCAATTGGAGCCC
GAAAGTAGTAGAGTTGTGCAAAAAGTACCAACAGCAAACTGTCGTTGCAATCGATCT
GGCGGGTGACGAAACGATTCCGGGGAGTTCTTTACTGCCAGGACACGTGCAGGCCTA
TCAGGAAGCGGTGAAATCCGGCATCCATCGTACGGTTCACGCAGGTGAGGTCGGCTC
AGCCGAAGTGGTTAAAGAGGCGGTGGATATTCTGAAAACCGAACGCCTCGGCCACG
GTTACCACACCTTGGAAGACCAAGCACTGTATAACCGTCTGCGCCAGGAGAATATGC
ATTTTGAAATCTGCCCCTGGAGCAGTTACTTAACTGGCGCCTGGAAACCTGACACAG
AACATGCAGTGATTCGTTTGAAAAACGATCAGGCGAATTATTCTCTTAACACGGATGA
TCCGCTGATATTCAAATCCACCCTGGATACCGACTACCAGATGACCAAACGCGATATG
GGGTTTACAGAGGAAGAATTTAAACGTCTGAATATTAACGCGGCGAAATCAAGCTTTT
TGCCAGAGGATGAAAAGCGCGAACTTTTAGATCTGCTGTATAAGGCGTACGGTATGCC
GCCTTCGGCAAGCGCGGGCCAGAACTTGTAA
(Seq ID No. 238)
MGGHHHHHHGGAQTPAFDKPKVELHVHLDGSIKPETILYYGRRRGIALPANT
AEGLLNVIGMDKPLTLPDFLAKFDYYMPAIAGSREAIKRIAYEFVEMKAKEGVVYVEVR
YSPHLLANSKVEPIPWNQAEGDLTPDEVVALVGQGLQEGERDFGVKARSILCCMRHQP
NWSPKVVELCKKYQQQTVVAIDLAGDETIPGSSLLPGHVQAYQEAVKSGIHRTVHAGE
VGSAEVVKEAVDILKTERLGHGYHTLEDQALYNRLRQENMHFEICPWSSYLTGAWKPD
TEHAVIRLKNDQANYSLNTDDPLIFKSTLDTDYQMTKRDMGFTEEEFKRLNINAAKSSF
LPEDEKRELLDLLYKAYGMPPSASAGQNL
N-GG-6His-GG-MmADA:
(Seq ID No. 239)
ATGGGCGGTCATCATCACCACCATCACGGCGGCGCGCAGACGCCGGCATTCAATAAG
CCAAAAGTCGAGTTGCACGTACATCTGGACGGGGCCATAAAACCGGAAACTATCTTA
TACTTTGGAAAAAAGCGTGGTATTGCGCTGCCCGCAGATACCGTTGAGGAACTTCGC
AACATCATTGGCATGGATAAACCGCTGTCCCTTCCTGGGTTTTTAGCAAAATTCGATTA
CTATATGCCGGTGATCGCTGGTTGTCGTGAAGCGATTAAACGCATTGCCTACGAGTTT
GTTGAAATGAAGGCAAAAGAAGGTGTGGTCTATGTAGAGGTTCGTTATTCGCCACAC
CTGCTTGCCAATTCTAAAGTGGATCCTATGCCGTGGAACCAGACCGAAGGCGACGTT
ACACCAGATGATGTCGTGGACCTGGTAAATCAAGGTTTGCAGGAAGGCGAACAGGC
GTTCGGAATTAAAGTTCGCAGCATTCTGTGCTGTATGCGTCATCAACCGTCATGGAGT
CTTGAAGTGCTGGAGCTTTGCAAAAAGTACAACCAGAAAACGGTTGTGGCTATGGAT
CTGGCCGGCGATGAAACTATCGAAGGTAGCTCTTTATTTCCAGGCCACGTCGAAGCGT
ATGAGGGCGCTGTAAAAAATGGTATTCATCGCACCGTGCATGCGGGCGAAGTGGGTA
GTCCGGAAGTCGTACGTGAGGCCGTTGACATCCTGAAAACGGAACGCGTGGGGCAT
GGCTATCACACCATTGAAGATGAAGCACTGTACAACCGTCTCCTGAAAGAGAATATGC
ATTTCGAAGTGTGCCCGTGGAGCTCATATCTCACCGGTGCCTGGGATCCCAAAACAAC
GCACGCTGTTGTCCGCTTTAAAAACGACAAGGCGAATTATAGCTTGAACACCGATGA
CCCGCTGATATTTAAATCCACTCTGGATACAGATTACCAGATGACGAAGAAAGACATG
GGATTCACCGAAGAGGAATTTAAACGTCTCAATATCAACGCAGCGAAATCGTCTTTTC
TGCCTGAAGAGGAAAAGAAAGAACTGTTAGAGCGCTTGTATCGCGAATATCAATAA
(Seq ID No. 240)
MGGHHHHHHGGAQTPAFNKPKVELHVHLDGAIKPETILYFGKKRGIALPADT
VEELRNIIGMDKPLSLPGFLAKFDYYMPVIAGCREAIKRIAYEFVEMKAKEGVVYVEVR
YSPHLLANSKVDPMPWNQTEGDVTPDDVVDLVNQGLQEGEQAFGIKVRSILCCMRHQP
SWSLEVLELCKKYNQKTVVAMDLAGDETIEGSSLFPGHVEAYEGAVKNGIHRTVHAGE
VGSPEVVREAVDILKTERVGHGYHTIEDEALYNRLLKENMHFEVCPWSSYLTGAWDPK
TTHAVVRFKNDKANYSLNTDDPLIFKSTLDTDYQMTKKDMGFTEEEFKRLNINAAKSSF
LPEEEKKELLERLYREYQ
N-GG-6His-GG-MmADAC57S:
(Seq ID No. 241)
ATGGGCGGTCATCATCACCACCATCACGGCGGCGCGCAGACGCCGGCATTCAATAAG
CCAAAAGTCGAGTTGCACGTACATCTGGACGGGGCCATAAAACCGGAAACTATCTTA
TACTTTGGAAAAAAGCGTGGTATTGCGCTGCCCGCAGATACCGTTGAGGAACTTCGC
AACATCATTGGCATGGATAAACCGCTGTCCCTTCCTGGGTTTTTAGCAAAATTCGATTA
CTATATGCCGGTGATCGCTGGTTCTCGTGAAGCGATTAAACGCATTGCCTACGAGTTT
GTTGAAATGAAGGCAAAAGAAGGTGTGGTCTATGTAGAGGTTCGTTATTCGCCACAC
CTGCTTGCCAATTCTAAAGTGGATCCTATGCCGTGGAACCAGACCGAAGGCGACGTT
ACACCAGATGATGTCGTGGACCTGGTAAATCAAGGTTTGCAGGAAGGCGAACAGGC
GTTCGGAATTAAAGTTCGCAGCATTCTGTGCTGTATGCGTCATCAACCGTCATGGAGT
CTTGAAGTGCTGGAGCTTTGCAAAAAGTACAACCAGAAAACGGTTGTGGCTATGGAT
CTGGCCGGCGATGAAACTATCGAAGGTAGCTCTTTATTTCCAGGCCACGTCGAAGCGT
ATGAGGGCGCTGTAAAAAATGGTATTCATCGCACCGTGCATGCGGGCGAAGTGGGTA
GTCCGGAAGTCGTACGTGAGGCCGTTGACATCCTGAAAACGGAACGCGTGGGGCAT
GGCTATCACACCATTGAAGATGAAGCACTGTACAACCGTCTCCTGAAAGAGAATATGC
ATTTCGAAGTGTGCCCGTGGAGCTCATATCTCACCGGTGCCTGGGATCCCAAAACAAC
GCACGCTGTTGTCCGCTTTAAAAACGACAAGGCGAATTATAGCTTGAACACCGATGA
CCCGCTGATATTTAAATCCACTCTGGATACAGATTACCAGATGACGAAGAAAGACATG
GGATTCACCGAAGAGGAATTTAAACGTCTCAATATCAACGCAGCGAAATCGTCTTTTC
TGCCTGAAGAGGAAAAGAAAGAACTGTTAGAGCGCTTGTATCGCGAATATCAATAA
(Seq ID No. 242)
MGGHHHHHHGGAQTPAFNKPKVELHVHLDGAIKPETILYFGKKRGIALPADT
VEELRNIIGMDKPLSLPGFLAKFDYYMPVIAGSREAIKRIAYEFVEMKAKEGVVYVEVR
YSPHLLANSKVDPMPWNQTEGDVTPDDVVDLVNQGLQEGEQAFGIKVRSILCCMRHQP
SWSLEVLELCKKYNQKTVVAMDLAGDETIEGSSLFPGHVEAYEGAVKNGIHRTVHAGE
VGSPEVVREAVDILKTERVGHGYHTIEDEALYNRLLKENMHFEVCPWSSYLTGAWDPK
TTHAVVRFKNDKANYSLNTDDPLIFKSTLDTDYQMTKKDMGFTEEEFKRLNINAAKSSF
LPEEEKKELLERLYREYQ
BovineHomologNucleotideandaminoacidsequencesusedtopurifyandcompare
HsADA1toBTADA1
BtADA:
(Seq ID No. 243)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTGTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGTAA
(Seq ID No. 244)
MAQTPAFNKPKVELHVHLDGAIKPETILYYGRKRGIALPADTPEELQNIIGMD
KPLSLPEFLAKFDYYMPAIAGCREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVVKEA
VDTLKTERLGHGYHTLEDATLYNRLRQENMHFEVCPWSSYLTGAWKPDTEHPVVRFK
NDQVNYSLNTDDPLIFKSTLDTDYQMTKNEMGFTEEEFKRLNINAAKSSFLPEDEKKEL
LDLLYKAYGMPSPASAEQCL
BtADAC75S:
(Seq ID No. 245)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTCTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGTAA
(Seq ID No. 246)
MAQTPAFNKPKVELHVHLDGAIKPETILYYGRKRGIALPADTPEELQNIIGMD
KPLSLPEFLAKFDYYMPAIAGSREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVVKEA
VDTLKTERLGHGYHTLEDATLYNRLRQENMHFEVCPWSSYLTGAWKPDTEHPVVRFK
NDQVNYSLNTDDPLIFKSTLDTDYQMTKNEMGFTEEEFKRLNINAAKSSFLPEDEKKEL
LDLLYKAYGMPSPASAEQCL
BtADAC57S-GG-6His-GG:
(Seq ID No. 247)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTCTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGGGC
GGTCATCATCACCACCATCACGGCGGCTAA
(Seq ID No. 248)
MAQTPAFNKPKVELHVHLDGAIKPETILYYGRKRGIALPADTPEELQNIIGMD
KPLSLPEFLAKFDYYMPAIAGSREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVVKEA
VDTLKTERLGHGYHTLEDATLYNRLRQENMHFEVCPWSSYLTGAWKPDTEHPVVRFK
NDQVNYSLNTDDPLIFKSTLDTDYQMTKNEMGFTEEEFKRLNINAAKSSFLPEDEKKEL
LDLLYKAYGMPSPASAEQCLGGHHHHHHGG
BtADA-GG-6His-GG:
(Seq ID No. 249)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTGTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGGGC
GGTCATCATCACCACCATCACGGCGGCTAA
(Seq ID No. 250)
MAQTPAFNKPKVELHVHLDGAIKPETILYYGRKRGIALPADTPEELQNIIGMD
KPLSLPEFLAKFDYYMPAIAGCREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVVKEA
VDTLKTERLGHGYHTLEDATLYNRLRQENMHFEVCPWSSYLTGAWKPDTEHPVVRFK
NDQVNYSLNTDDPLIFKSTLDTDYQMTKNEMGFTEEEFKRLNINAAKSSFLPEDEKKEL
LDLLYKAYGMPSPASAEQCLGGHHHHHHGG
BtADA-GG-6His-GG-6His-GG:
(Seq ID No. 251)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTGTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGGGC
GGTCATCATCACCACCATCACGGCGGCCATCACCATCACCATCACGGGGGTTAA
(Seq ID No. 252)
MAQTPAFNKPKVELHVHLDGAIKPETILYYGRKRGIALPADTPEELQNIIGMD
KPLSLPEFLAKFDYYMPAIAGCREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVVKEA
VDTLKTERLGHGYHTLEDATLYNRLRQENMHFEVCPWSSYLTGAWKPDTEHPVVRFK
NDQVNYSLNTDDPLIFKSTLDTDYQMTKNEMGFTEEEFKRLNINAAKSSFLPEDEKKEL
LDLLYKAYGMPSPASAEQCLGGHHHHHHGGHHHHHHGG
BtADA-GG-6His-GG-10His-GG:
(Seq ID No. 253)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTGTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGGGC
GGTCATCATCACCACCATCACGGCGGCCATCACCATCACCATCACCATCACCATCACG
GGGGTTAA
(Seq ID No. 254)
MAQTPAFNKPKVELHVHLDGAIKPETILYYGRKRGIALPADTPEELQNIIGMD
KPLSLPEFLAKFDYYMPAIAGCREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVVKEA
VDTLKTERLGHGYHTLEDATLYNRLRQENMHFEVCPWSSYLTGAWKPDTEHPVVRFK
NDQVNYSLNTDDPLIFKSTLDTDYQMTKNEMGFTEEEFKRLNINAAKSSFLPEDEKKEL
LDLLYKAYGMPSPASAEQCLGGHHHHHHGGHHHHHHHHHHGG
BtADA-TEV-GG-6His-GG-10His-GG:
(Seq ID No. 255)
ATGGCTCAAACTCCCGCCTTCAATAAGCCTAAAGTCGAGCTCCACGTTCATCTGGACG
GGGCGATAAAACCAGAAACAATCCTGTACTACGGTCGCAAACGTGGGATTGCCCTGC
CGGCAGATACGCCGGAAGAGCTGCAGAACATCATTGGCATGGATAAGCCGCTGAGTC
TTCCTGAATTTTTGGCTAAATTCGACTACTATATGCCCGCCATCGCAGGTTGTCGCGAA
GCGGTGAAACGTATTGCGTACGAGTTTGTAGAAATGAAAGCTAAGGATGGCGTGGTC
TATGTTGAAGTGCGCTACTCACCACACCTCTTAGCAAATAGCAAAGTAGAGCCGATCC
CGTGGAACCAGGCCGAAGGCGACCTGACCCCGGATGAAGTCGTGTCCCTGGTTAATC
AAGGTCTGCAGGAGGGCGAACGTGATTTCGGCGTGAAAGTACGCAGTATATTATGCT
GTATGCGTCATCAGCCTTCGTGGAGCTCCGAAGTCGTTGAGCTTTGCAAAAAGTATCG
CGAACAAACCGTTGTGGCGATCGACCTCGCAGGTGATGAAACTATTGAGGGCAGTTC
TTTGTTTCCAGGCCACGTCAAAGCTTACGCGGAAGCGGTTAAATCAGGAGTGCATCG
TACAGTGCACGCCGGTGAAGTGGGCTCGGCAAACGTCGTAAAAGAGGCGGTTGATA
CGTTAAAGACCGAACGCCTGGGACATGGTTATCACACCCTGGAAGACGCAACGCTGT
ACAATCGTCTGCGCCAGGAGAACATGCATTTCGAAGTGTGTCCCTGGAGCTCGTATCT
CACTGGAGCCTGGAAACCGGATACAGAACATCCGGTGGTTCGTTTTAAAAATGACCA
GGTAAACTATTCTCTTAATACGGATGATCCGTTGATTTTCAAATCAACCTTAGACACCG
ATTATCAAATGACGAAGAACGAGATGGGGTTTACCGAAGAAGAGTTTAAACGCCTGA
ATATTAACGCGGCGAAAAGCAGCTTTCTGCCTGAAGATGAAAAAAAGGAATTACTGG
ATCTGCTTTATAAAGCGTATGGTATGCCATCTCCGGCCTCCGCTGAACAGTGCTTGGA
GAACCTGTACTTCCAATCCGGCGGTCATCATCACCACCATCACGGCGGCCATCACCAT
CACCATCACCATCACCATCACGGGGGTTAA
(Seq ID No. 256)
MAQTPAFNKPKVELHVHLDGAIKPETILYYGRKRGIALPADTPEELQNIIGMD
KPLSLPEFLAKFDYYMPAIAGCREAVKRIAYEFVEMKAKDGVVYVEVRYSPHLLANSK
VEPIPWNQAEGDLTPDEVVSLVNQGLQEGERDFGVKVRSILCCMRHQPSWSSEVVELC
KKYREQTVVAIDLAGDETIEGSSLFPGHVKAYAEAVKSGVHRTVHAGEVGSANVKEAV
DTLKTERLGHGYHTLEDATLYNRLRQENMHFEVCPWSSYLTGAWKPDTEHPVVRFKN
DQVNYSLNTDDPLIFKSTLDTDYQMTKNEMGFTEEEFKRLNINAAKSSFLPEDEKKELL
DLLYKAYGMPSPASAEQCLENLYFQSGGHHHHHHGGHHHHHHHHHHGG
GST-Thrombin-HsADA1(codonopt1)-GG-6His-GG:
(Seq ID No. 257)
ATGTCCCCTATACTAGGTTATTGGAAAATTAAGGGCCTTGTGCAACCCACTCGACTTCT
TTTGGAATATCTTGAAGAAAAATATGAAGAGCATTTGTATGAGCGCGATGAAGGTGAT
AAATGGCGAAACAAAAAGTTTGAATTGGGTTTGGAGTTTCCCAATCTTCCTTATTATAT
TGATGGTGATGTTAAATTAACACAGTCTATGGCCATCATACGTTATATAGCTGACAAGC
ACAACATGTTGGGTGGTTGTCCAAAAGAGCGTGCAGAGATTTCAATGCTTGAAGGAG
CGGTTTTGGATATTAGATACGGTGTTTCGAGAATTGCATATAGTAAAGACTTTGAAACT
CTCAAAGTTGATTTTCTTAGCAAGCTACCTGAAATGCTGAAAATGTTCGAAGATCGTT
TATGTCATAAAACATATTTAAATGGTGATCATGTAACCCATCCTGACTTCATGTTGTATG
ACGCTCTTGATGTTGTTTTATACATGGACCCAATGTGCCTGGATGCGTTCCCAAAATTA
GTTTGTTTTAAAAAACGTATTGAAGCTATCCCACAAATTGATAAGTACTTGAAATCCA
GCAAGTATATAGCATGGCCTTTGCAGGGCTGGCAAGCCACGTTTGGTGGTGGCGACC
ATCCTCCAAAATCGGATCTGGTTCCGCGTGGATCCCCAATGGCTCAAACTCCGGCCTT
CGACAAGCCGAAAGTCGAACTCCATGTACATCTTGATGGAAGTATAAAACCGGAGAC
GATCCTGTATTATGGGCGCCGTCGCGGTATTGCTTTACCTGCCAATACCGCAGAAGGTC
TGCTGAACGTTATCGGCATGGACAAACCCCTCACCCTTCCGGATTTTCTGGCTAAGTT
CGATTATTACATGCCGGCGATTGCTGGTTGTCGTGAAGCCATCAAACGCATTGCTTATG
AGTTTGTTGAAATGAAAGCGAAAGAAGGCGTGGTCTACGTTGAAGTACGTTATTCTC
CTCACCTGTTAGCTAATTCCAAGGTGGAGCCAATCCCGTGGAACCAAGCCGAAGGAG
ATCTGACACCAGACGAAGTTGTCGCACTGGTGGGCCAGGGTCTCCAGGAGGGGGAA
CGCGACTTCGGCGTGAAAGCCCGTTCGATTCTTTGCTGTATGCGCCATCAGCCCAATT
GGAGCCCGAAAGTAGTAGAGTTGTGCAAAAAGTACCAACAGCAAACTGTCGTTGCA
ATCGATCTGGCGGGTGACGAAACGATTCCGGGGAGTTCTTTACTGCCAGGACACGTG
CAGGCCTATCAGGAAGCGGTGAAATCCGGCATCCATCGTACGGTTCACGCAGGTGAG
GTCGGCTCAGCCGAAGTGGTTAAAGAGGCGGTGGATATTCTGAAAACCGAACGCCTC
GGCCACGGTTACCACACCTTGGAAGACCAAGCACTGTATAACCGTCTGCGCCAGGAG
AATATGCATTTTGAAATCTGCCCCTGGAGCAGTTACTTAACTGGCGCCTGGAAACCTG
ACACAGAACATGCAGTGATTCGTTTGAAAAACGATCAGGCGAATTATTCTCTTAACAC
GGATGATCCGCTGATATTCAAATCCACCCTGGATACCGACTACCAGATGACCAAACGC
GATATGGGGTTTACAGAGGAAGAATTTAAACGTCTGAATATTAACGCGGCGAAATCAA
GCTTTTTGCCAGAGGATGAAAAGCGCGAACTTTTAGATCTGCTGTATAAGGCGTACGG
TATGCCGCCTTCGGCAAGCGCGGGCCAGAACTTGGGCGGTCATCATCACCACCATCA
CGGCGGCTAA
(Seq ID No. 258)
MSPILGYWKIKGLVQPTRLLLEYLEEKYEEHLYERDEGDKWRNKKFELGLEF
PNLPYYIDGDVKLTQSMAIIRYIADKHNMLGGCPKERAEISMLEGAVLDIRYGVSRIAYS
KDFETLKVDFLSKLPEMLKMFEDRLCHKTYLNGDHVTHPDFMLYDALDVVLYMDPMC
LDAFPKLVCFKKRIEAIPQIDKYLKSSKYIAWPLQGWQATFGGGDHPPKSDLVPRGSPM
AQTPAFDKPKVELHVHLDGSIKPETILYYGRRRGIALPANTAEGLLNVIGMDKPLTLPDF
LAKFDYYMPAIAGCREAIKRIAYEFVEMKAKEGVVYVEVRYSPHLLANSKVEPIPWNQ
AEGDLTPDEVVALVGQGLQEGERDFGVKARSILCCMRHQPNWSPKVVELCKKYQQQT
VVAIDLAGDETIPGSSLLPGHVQAYQEAVKSGIHRTVHAGEVGSAEVVKEAVDILKTER
LGHGYHTLEDQALYNRLRQENMHFEICPWSSYLTGAWKPDTEHAVIRLKNDQANYSLN
TDDPLIFKSTLDTDYQMTKRDMGFTEEEFKRLNINAAKSSFLPEDEKRELLDLLYKAYG
MPPSASAGQNLGGHHHHHHGG
MurineHomologNucleotideandaminoacidsequencesusedtopurifyandcompare
HSADA1toMMADA1
MmADA:
(Seq ID No. 259)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTGTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAATAA
(Seq ID No. 260)
MAQTPAFNKPKVELHVHLDGAIKPETILYFGKKRGIALPADTVEELRNIIGMD
KPLSLPGFLAKFDYYMPVIAGCREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQ
MmADAC57S:
(Seq ID No. 261)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTCTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAATAA
(Seq ID No. 262)
MAQTPAFNKPKVELHVHLDGAIKPETILYFGKKRGIALPADTVEELRNIIGMD
KPLSLPGFLAKFDYYMPVIAGSREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQ
MmADAC57S-GG-6His-GG:
(Seq ID No. 263)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTCTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAAGGCGGTCATCATCACCACCATCACGGCGGCTAA
(Seq ID No. 264)
MAQTPAFNKPKVELHVHLDGAIKPETILYFGKKRGIALPADTVEELRNIIGMD
KPLSLPGFLAKFDYYMPVIAGSREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQGGHHHHHHGG
MmADA-GG-6His-GG:
(Seq ID No. 265)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTGTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAAGGCGGTCATCATCACCACCATCACGGCGGCTAA
(Seq ID No. 266)
MAQTPAFNKPKVELHVHLDGAIKPETILYFGKKRGIALPADTVEELRNIIGMD
KPLSLPGFLAKFDYYMPVIAGCREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQGGHHHHHHGG
MmADA-GG-6His-GG-10His-GG:
(Seq ID No. 267)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTGTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAAGGCGGTCATCATCACCACCATCACGGCGGCCATCACC
ATCACCATCACCATCACCATCACGGGGGTTAA
(Seq ID No. 268)
MAQTPAFNKPKVELHVHLDGAIKPETILYFGKKRGIALPADTVEELRNIIGMD
KPLSLPGFLAKFDYYMPVIAGCREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQGGHHHHHHGGHHHHHHHHHHGG
MmADA-GG-6His-GG-6His-GG:
(Seq ID No. 269)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTGTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAAGGCGGTCATCATCACCACCATCACGGCGGCCATCACC
ATCACCATCACGGGGGTTAA
(Seq ID No. 270)
MAQTPAFNKPKVELHVHLDGAIKPETILYFGKKRGIALPADTVEELRNIIGMD
KPLSLPGFLAKFDYYMPVIAGCREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQGGHHHHHHGGHHHHHHGG
MmADA-TEV-GG-6His-GG-10His-GG:
(Seq ID No. 271)
ATGGCGCAGACGCCGGCATTCAATAAGCCAAAAGTCGAGTTGCACGTACATCTGGAC
GGGGCCATAAAACCGGAAACTATCTTATACTTTGGAAAAAAGCGTGGTATTGCGCTGC
CCGCAGATACCGTTGAGGAACTTCGCAACATCATTGGCATGGATAAACCGCTGTCCCT
TCCTGGGTTTTTAGCAAAATTCGATTACTATATGCCGGTGATCGCTGGTTGTCGTGAAG
CGATTAAACGCATTGCCTACGAGTTTGTTGAAATGAAGGCAAAAGAAGGTGTGGTCT
ATGTAGAGGTTCGTTATTCGCCACACCTGCTTGCCAATTCTAAAGTGGATCCTATGCCG
TGGAACCAGACCGAAGGCGACGTTACACCAGATGATGTCGTGGACCTGGTAAATCAA
GGTTTGCAGGAAGGCGAACAGGCGTTCGGAATTAAAGTTCGCAGCATTCTGTGCTGT
ATGCGTCATCAACCGTCATGGAGTCTTGAAGTGCTGGAGCTTTGCAAAAAGTACAAC
CAGAAAACGGTTGTGGCTATGGATCTGGCCGGCGATGAAACTATCGAAGGTAGCTCT
TTATTTCCAGGCCACGTCGAAGCGTATGAGGGCGCTGTAAAAAATGGTATTCATCGCA
CCGTGCATGCGGGCGAAGTGGGTAGTCCGGAAGTCGTACGTGAGGCCGTTGACATCC
TGAAAACGGAACGCGTGGGGCATGGCTATCACACCATTGAAGATGAAGCACTGTACA
ACCGTCTCCTGAAAGAGAATATGCATTTCGAAGTGTGCCCGTGGAGCTCATATCTCAC
CGGTGCCTGGGATCCCAAAACAACGCACGCTGTTGTCCGCTTTAAAAACGACAAGGC
GAATTATAGCTTGAACACCGATGACCCGCTGATATTTAAATCCACTCTGGATACAGATT
ACCAGATGACGAAGAAAGACATGGGATTCACCGAAGAGGAATTTAAACGTCTCAATA
TCAACGCAGCGAAATCGTCTTTTCTGCCTGAAGAGGAAAAGAAAGAACTGTTAGAG
CGCTTGTATCGCGAATATCAAGAGAACCTGTACTTCCAATCCGGCGGTCATCATCACC
ACCATCACGGCGGCCATCACCATCACCATCACCATCACCATCACGGGGGTTAA
(Seq ID No. 272)
MAQTPAFNKPKVELHVHLDGAIKPETILYFGKKRGIALPADTVEELRNIIGMD
KPLSLPGFLAKFDYYMPVIAGCREAIKRIAYEFVEMKAKEGVVYVEVRYSPHLLANSKV
DPMPWNQTEGDVTPDDVVDLVNQGLQEGEQAFGIKVRSILCCMRHQPSWSLEVLELCK
KYNQKTVVAMDLAGDETIEGSSLFPGHVEAYEGAVKNGIHRTVHAGEVGSPEVVREAV
DILKTERVGHGYHTIEDEALYNRLLKENMHFEVCPWSSYLTGAWDPKTTHAVVRFKND
KANYSLNTDDPLIFKSTLDTDYQMTKKDMGFTEEEFKRLNINAAKSSFLPEEEKKELLER
LYREYQENLYFQSGGHHHHHHGGHHHHHHHHHHGG
IGGSIGNALPEPTIDE
(Seq ID No. 273)
ATGCGGCCTACTTGGGCCTGGTGGCTGTTCCTGGTGCTGCTGCTAGCTCTG
TGGGCACCCGCTAGAGGG
(Seq ID No. 274)
MRPTWAWWLFLVLLLALWAPARG
GLYCINESERINELINKER
(Seq ID No. 275)
GGAGGAGGTGGCTCTGGCGGTGGAGGTTCC
(Seq ID No. 276)
GGGGSGGGGS
C-TERMINAL6XHISTAG
(Seq ID No. 277)
GGCGGACACCACCATCACCACCATGGAGGC
(Seq ID No. 278)
GGHHHHHHGG
HSADA1CDSUNMODIFIED
(Seq ID No. 279)
ATGGCCCAGACGCCCGCCTTCGACAAGCCCAAAGTGGAACTGCATGTCCA
CCTAGACGGATCCATCAAGCCTGAAACCATCTTATACTATGGCAGGAGGAGAGGGA
TCGCCCTCCCAGCTAACACAGCAGAGGGGCTGCTGAACGTCATTGGCATGGACAAG
CCGCTCACCCTTCCAGACTTCCTGGCCAAGTTTGACTACTACATGCCTGCTATCGCG
GGCTGCCGGGAGGCTATCAAAAGGATCGCCTATGAGTTTGTAGAGATGAAGGCCAA
AGAGGGCGTGGTGTATGTGGAGGTGCGGTACAGTCCGCACCTGCTGGCCAACTCCA
AAGTGGAGCCAATCCCCTGGAACCAGGCTGAAGGGGACCTCACCCCAGACGAGGTG
GTGGCCCTAGTGGGCCAGGGCCTGCAGGAGGGGGAGCGAGACTTCGGGGTCAAGGC
CCGGTCCATCCTGTGCTGCATGCGCCACCAGCCCAACTGGTCCCCCAAGGTGGTGGA
GCTGTGTAAGAAGTACCAGCAGCAGACCGTGGTAGCCATTGACCTGGCTGGAGATG
AGACCATCCCAGGAAGCAGCCTCTTGCCTGGACATGTCCAGGCCTACCAGGAGGCT
GTGAAGAGCGGCATTCACCGTACTGTCCACGCCGGGGAGGTGGGCTCGGCCGAAGT
AGTAAAAGAGGCTGTGGACATACTCAAGACAGAGCGGCTGGGACACGGCTACCACA
CCCTGGAAGACCAGGCCCTTTATAACAGGCTGCGGCAGGAAAACATGCACTTCGAG
ATCTGCCCCTGGTCCAGCTACCTCACTGGTGCCTGGAAGCCGGACACGGAGCATGCA
GTCATTCGGCTCAAAAATGACCAGGCTAACTACTCGCTCAACACAGATGACCCGCTC
ATCTTCAAGTCCACCCTGGACACTGATTACCAGATGACCAAACGGGACATGGGCTTT
ACTGAAGAGGAGTTTAAAAGGCTGAACATCAATGCGGCCAAATCTAGTTTCCTCCCA
GAAGATGAAAAGAGGGAGCTTCTCGACCTGCTCTATAAAGCCTATGGGATGCCACC
TTCAGCCTCTGCAGGGCAGAACCT
(Seq ID No. 280)
AQTPAFDKPKVELHVHLDGSIKPETILYYGRRRGIALPANTAEGLLNVIGMDK
PLTLPDFLAKFDYYMPAIAGCREAIKRIAYEFVEMKAKEGVVYVEVRYSPHLLANSKVE
PIPWNQAEGDLTPDEVVALVGQGLQEGERDFGVKARSILCCMRHQPNWSPKVVELCKK
YQQQTVVAIDLAGDETIPGSSLLPGHVQAYQEAVKSGIHRTVHAGEVGSAEVVKEAVDI
LKTERLGHGYHTLEDQALYNRLRQENMHFEICPWSSYLTGAWKPDTEHAVIRLKNDQA
NYSLNTDDPLIFKSTLDTDYQMTKRDMGFTEEEFKRLNINAAKSSFLPEDEKRELLDLLY
KAYGMPPSASAGQN
MONOMERICFCDOMAIN
(Seq ID No. 281)
ACTCACACATCCCCACCGAGCCCAGCACCTGAACTCCTGGGGGGACCGTC
AGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACACCAA
ACCCCCATCCCGGGATGAGCTGACCAAGAACCAGGTCAGCCTGTCCTGCCTGGTCA
AAGGCTTCTATCCCAGCGACATCGCCGTGGAGTGGGAGAGCAATGGGCAGCCGGAG
AACAACTACAAGACCACGGTCCCGTGCTGGACTCCGACGGCTCCTTCAGGCTCGCCA
GCTATCTCACCGTGGACAAGAGCAGGTGGCAGCAGGGGAACGTCTTCTCATGCTCC
GTGATGCATGAGGCTCTGCACAACCACTACACGCAGAAGAGCCTCTCCCTGTCTCCG
GGTAAA
(Seq ID No. 282)
THTSPPSPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIE
KTISKAKGQPREPQVYTKPPSRDELTKNQVSLSCLVKGFYPSDIAVEWESNGQPENNYK
TTVPVLDSDGSFRLASYLTVDKSRWQQGNVFSCSVMHEALHNHYTQKSLSLSPGK
IGG-HSADA1-GS-FC I IGG SIGNAL PEPTIDE PRECEDING HSADA1
WITHOUT INITIATING METHIONINE FUSED TO A MONOMERIC IGG FC DOMAIN BY
A GLYCINE SERINE LINKER WITH A C-TERMINAL 6XHIS TAG
(Seq ID No. 283)
ATGCGGCCTACTTGGGCCTGGTGGCTGTTCCTGGTGCTGCTGCTAGCTCTG
TGGGCACCCGCTAGAGGGGCCCAGACGCCCGCCTTCGACAAGCCCAAAGTGGAACT
GCATGTCCACCTAGACGGATCCATCAAGCCTGAAACCATCTTATACTATGGCAGGAG
GAGAGGGATCGCCCTCCCAGCTAACACAGCAGAGGGGCTGCTGAACGTCATTGGCA
TGGACAAGCCGCTCACCCTTCCAGACTTCCTGGCCAAGTTTGACTACTACATGCCTG
CTATCGCGGGCTGCCGGGAGGCTATCAAAAGGATCGCCTATGAGTTTGTAGAGATG
AAGGCCAAAGAGGGCGTGGTGTATGTGGAGGTGCGGTACAGTCCGCACCTGCTGGC
CAACTCCAAAGTGGAGCCAATCCCCTGGAACCAGGCTGAAGGGGACCTCACCCCAG
ACGAGGTGGTGGCCCTAGTGGGCCAGGGCCTGCAGGAGGGGGAGCGAGACTTCGGG
GTCAAGGCCCGGTCCATCCTGTGCTGCATGCGCCACCAGCCCAACTGGTCCCCCAAG
GTGGTGGAGCTGTGTAAGAAGTACCAGCAGCAGACCGTGGTAGCCATTGACCTGGC
TGGAGATGAGACCATCCCAGGAAGCAGCCTCTTGCCTGGACATGTCCAGGCCTACC
AGGAGGCTGTGAAGAGCGGCATTCACCGTACTGTCCACGCCGGGGAGGTGGGCTCG
GCCGAAGTAGTAAAAGAGGCTGTGGACATACTCAAGACAGAGCGGCTGGGACACG
GCTACCACACCCTGGAAGACCAGGCCCTTTATAACAGGCTGCGGCAGGAAAACATG
CACTTCGAGATCTGCCCCTGGTCCAGCTACCTCACTGGTGCCTGGAAGCCGGACACG
GAGCATGCAGTCATTCGGCTCAAAAATGACCAGGCTAACTACTCGCTCAACACAGA
TGACCCGCTCATCTTCAAGTCCACCCTGGACACTGATTACCAGATGACCAAACGGGA
CATGGGCTTTACTGAAGAGGAGTTTAAAAGGCTGAACATCAATGCGGCCAAATCTA
GTTTCCTCCCAGAAGATGAAAAGAGGGAGCTTCTCGACCTGCTCTATAAAGCCTATG
GGATGCCACCTTCAGCCTCTGCAGGGCAGAACCTCGGAGGAGGTGGCTCTGGCGGT
GGAGGTTCCACTCACACATCCCCACCGAGCCCAGCACCTGAACTCCTGGGGGGACC
GTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTACACC
AAACCCCCATCCCGGGATGAGCTGACCAAGAACCAGGTCAGCCTGTCCTGCCTGGT
CAAAGGCTTCTATCCCAGCGACATCGCCGTGGAGTGGGAGAGCAATGGGCAGCCGG
AGAACAACTACAAGACCACGGTCCCCGTGCTGGACTCCGACGGCTCCTTCAGGCTC
GCCAGCTATCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTC
TCCGGGTAAAGGCGGACACCACCATCACCACCATGGAGGC
(Seq ID No. 284)
MRPTWAWWLFLVLLLALWAPARGAQTPAFDKPKVELHVHLDGSIKPETILY
YGRRRGIALPANTAEGLLNVIGMDKPLTLPDFLAKFDYYMPAIAGCREAIKRIAYEFVE
MKAKEGVVYVEVRYSPHLLANSKVEPIPWNQAEGDLTPDEVVALVGQGLQEGERDFG
VKARSILCCMRHQPNWSPKVVELCKKYQQQTVVAIDLAGDETIPGSSLLPGHVQAYQE
AVKSGIHRTVHAGEVGSAEVVKEAVDILKTERLGHGYHTLEDQALYNRLRQENMHFEI
CPWSSYLTGAWKPDTEHAVIRLKNDQANYSLNTDDPLIFKSTLDTDYQMTKRDMGFTE
EEFKRLNINAAKSSFLPEDEKRELLDLLYKAYGMPPSASAGQNLGGGGSGGGGSTHTSP
PSPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTKPPSRDELTKNQVSLSCLVKGFYPSDIAVEWESNGQPENNYKTTVPVLDSDGS
FRLASYLTVDKSRWQQGNVFSCSVMHEALHNHYTQKSLSLSPGKGGHHHHHHGG
IGG-FC-GS-HSADA1 I IGG SIGNAL PEPTIDE PRECEDING A MONOMERIC
IGG FC DOMAIN FUSED TO ADAI BY A GLYCINE SERINE LINKER WITH A C-
TERMINAL 6XHIS TAG
(Seq ID No. 285)
ATGCGGCCTACTTGGGCCTGGTGGCTGTTCCTGGTGCTGCTGCTAGCTCTG
TGGGCACCCGCTAGAGGGACTCACACATCCCCACCGAGCCCAGCACCTGAACTCCT
GGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCG
CGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACCACAG
GTGTACACCAAACCCCCATCCCGGGATGAGCTGACCAAGAACCAGGTCAGCCTGTC
CTGCCTGGTCAAAGGCTTCTATCCCAGCGACATCGCCGTGGAGTGGGAGAGCAATG
GGCAGCCGGAGAACAACTACAAGACCACGGTCCCCGTGCTGGACTCCGACGGCTCC
TTCAGGCTCGCCAGCTATCTCACCGTGGACAAGAGCAGGTGGCAGCAGGGGAACGT
CTTCTCATGCTCCGTGATGCATGAGGCTCTGCACAACCACTACACGCAGAAGAGCCT
CTCCCTGTCTCCGGGTAAAGGAGGAGGTGGCTCTGGCGGTGGAGGTTCCGCCCAGA
CGCCCGCCTTCGACAAGCCCAAAGTGGAACTGCATGTCCACCTAGACGGATCCATC
AAGCCTGAAACCATCTTATACTATGGCAGGAGGAGAGGGATCGCCCTCCCAGCTAA
CACAGCAGAGGGGCTGCTGAACGTCATTGGCATGGACAAGCCGCTCACCCTTCCAG
ACTTCCTGGCCAAGTTTGACTACTACATGCCTGCTATCGCGGGCTGCCGGGAGGCTA
TCAAAAGGATCGCCTATGAGTTTGTAGAGATGAAGGCCAAAGAGGGCGTGGTGTAT
GTGGAGGTGCGGTACAGTCCGCACCTGCTGGCCAACTCCAAAGTGGAGCCAATCCC
CTGGAACCAGGCTGAAGGGGACCTCACCCCAGACGAGGTGGTGGCCCTAGTGGGCC
AGGGCCTGCAGGAGGGGGAGCGAGACTTCGGGGTCAAGGCCCGGTCCATCCTGTGC
TGCATGCGCCACCAGCCCAACTGGTCCCCCAAGGTGGTGGAGCTGTGTAAGAAGTA
CCAGCAGCAGACCGTGGTAGCCATTGACCTGGCTGGAGATGAGACCATCCCAGGAA
GCAGCCTCTTGCCTGGACATGTCCAGGCCTACCAGGAGGCTGTGAAGAGCGGCATT
CACCGTACTGTCCACGCCGGGGAGGTGGGCTCGGCCGAAGTAGTAAAAGAGGCTGT
GGACATACTCAAGACAGAGCGGCTGGGACACGGCTACCACACCCTGGAAGACCAGG
CCCTTTATAACAGGCTGCGGCAGGAAAACATGCACTTCGAGATCTGCCCCTGGTCCA
GCTACCTCACTGGTGCCTGGAAGCCGGACACGGAGCATGCAGTCATTCGGCTCAAA
AATGACCAGGCTAACTACTCGCTCAACACAGATGACCCGCTCATCTTCAAGTCCACC
CTGGACACTGATTACCAGATGACCAAACGGGACATGGGCTTTACTGAAGAGGAGTT
TAAAAGGCTGAACATCAATGCGGCCAAATCTAGTTTCCTCCCAGAAGATGAAAAGA
GGGAGCTTCTCGACCTGCTCTATAAAGCCTATGGGATGCCACCTTCAGCCTCTGCAG
GGCAGAACCTCGGCGGACACCACCATCACCACCATGGAGGC
(Seq ID No. 286)
MRPTWAWWLFLVLLLALWAPARGTHTSPPSPAPELLGGPSVFLFPPKPKDTL
MISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTKPPSRDELTKNQVSLSCL
VKGFYPSDIAVEWESNGQPENNYKTTVPVLDSDGSFRLASYLTVDKSRWQQGNVFSCS
VMHEALHNHYTQKSLSLSPGKGGGGSGGGGSAQTPAFDKPKVELHVHLDGSIKPETILY
YGRRRGIALPANTAEGLLNVIGMDKPLTLPDFLAKFDYYMPAIAGCREAIKRIAYEFVE
MKAKEGVVYVEVRYSPHLLANSKVEPIPWNQAEGDLTPDEVVALVGQGLQEGERDFG
VKARSILCCMRHQPNWSPKVVELCKKYQQQTVVAIDLAGDETIPGSSLLPGHVQAYQE
AVKSGIHRTVHAGEVGSAEVVKEAVDILKTERLGHGYHTLEDQALYNRLRQENMHFEI
CPWSSYLTGAWKPDTEHAVIRLKNDQANYSLNTDDPLIFKSTLDTDYQMTKRDMGFTE
EEFKRLNINAAKSSFLPEDEKRELLDLLYKAYGMPPSASAGQNLGGHHHHHHGG
(Seq ID No. 287)
5′-AACTTTAAGAAGGAGATATACCATGGCTCAAACTCCGGCCTTCGAC
(Seq ID No. 288)
5′-TGGTGGTGATGATGACCGCCCAAGTTCTGGCCCGCGCTTG
(Seq ID No. 289)
5′-CGAGTGCGGCCGCAAGCTTGTCGACTTAGCCGCCGTGATGGTGGTGATGATGAC-
CGCC
(Seq ID No. 290)
MAQTPAFDKPKVELHVHLDGSIKPETILYYGRRRGIALPANTAEGLLNVIGM
DKPLTLPDFLAKFDYYMPAIAGCREAIKRIAYEFVEMKAKEGVVYVEVRYSPHLLANSK
VEPIPWNQAEGDLTPDEVVALVGQGLQEGERDFGVKARSILCCMRHQPNWSPKVVELC
KKYQQQTVVAIDLAGDETIPGSSLLPGHVQAYQEAVKSGIHRTVHAGEVGSAEVVKEA
VDILKTERLGHGYHTLEDQALYNRLRQENMHFEICPWSSYLTGAWKPDTEHAVIRLKN
DQANYSLNTDDPLIFKSTLDTDYQMTKRDMGFTEEEFKRLNINAAKSSFLPEDEKRELL
DLLYKAYGMPPSASAGQNLGGHHHHHHGG
(Seq ID No. 291)
5′-GGAATTGTGAGCGGATAACAATTCCCC
(Seq ID No. 292)
5′-CAGTGGTGGTGGTGGTGGTGGCCGCCGTGATGGTGGT
(Seq ID No. 293)
HsADA1-GGHHHHHHGGHHHHHH
(Seq ID No. 294)
5′-GGGGAATTGTGAGCGGATAACAATTCCCCTC
(Seq ID No. 295)
5′-GCCGCCGTGATGGTGGTGATGATGACCGCCGGATTGGAAGTACAGGTTCTCCAAGTT
CTGGCCCGCGCTTG
(Seq ID No. 296)
5′-GGCGGTCATCATCACCACCATCAC
(Seq ID No. 297)
5′-ACTGCGATCCCCGGGAAAAC
(Seq ID No. 298)
5′-CTTCTAATACCTGGAATGCT
(Seq ID No. 299)
5′-GGTATATCTCCTTCTTAAAGTTAAA
(SEQ ID NO: 300)
Gly-Gly-His-His-His-His-His-His-Gly-Gly
(SEQ ID NO: 301)
5′-TCGAGTGCGGCCGCAAGCTTGTCGACTTAGCCGCCGTGATGGTGGTGATGATGACCGCC

### List of Embodiments

The following is a non-exhaustive list of embodiments contemplated by the invention.

1. A nucleic acid molecule encoding human adenosine deaminase 1 (HsADA1), wherein the HsADA1 has been mutated to have increased or prolonged stability or catalytic activity at physiological conditions relative to a wildtype HsADA1.

2. The nucleic acid molecule of item 1, wherein the HsADA1 has been mutated at one or more amino acid positions comprising a charged amino acid to a neutral or differently charged amino acid.

3. The nucleic acid molecule of items 1 or 2, wherein the nucleic acid molecule comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

4. The nucleic acid molecule of any of items 1-3, wherein the at least one mutation is in positions D8, S21, R33, N41, G45, V49, T57, G134, A148, N160, P163, K164, Q173, P189, L194, Q199, Q202, 1261, 1281, L283, and/or R341 of SEQ ID NO: 280.

5. The nucleic acid molecule of any of items 1-4, wherein the at least one mutation alters the charge of one or more of positions D8, S21, R33, N41, G45, V49, T57, G134, A148, N160, P163, K164, Q173, P189, L194, Q199, Q202, 1261, 1281, L283, and/or R341 of SEQ ID NO: 280.

6. The nucleic acid molecule of any of items 1-5, wherein the at least one mutation is selected from the group consisting of:

N at position D8; A at position S21; K at position R33; D at position N41; Eat position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; Vat position 1281; F at position L283; and K at position R341.

7. The nucleic acid molecule of any of items 1-6, wherein the at least one mutation comprises R33K, N41D, K164E, Q173N, L194F, Q199K, and/or Q202E.

8. The nucleic acid molecule of any of items 1-7, wherein the at least one mutation comprises one of (i) K164E, L194F, and Q199K; (ii) K164E, L194F, and Q173N; or (iii) K164E, L194F, and Q202E.

9. The nucleic acid molecule of any of items 1-8, further comprising at least one additional mutation in one or more of positions Y30, R32, D60, A71, E77, 179, El 13, 1115, Al20, L124, E128, A131, R142, D143, V146, V166, Q175, 1180, E203, S207, 1209, A221, E222, K225, 12301, L236, L243, Q246, A247, R253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, 1115M, Al20T, L124V, E128D, A131D or S, R142Q, D143A, V1461, V166L, Q175K, 1180M, E203G, 5207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

10. The nucleic acid molecule of any of items 1-9, further comprising at least one additional mutation in one or more of positions 160-202 of SEQ ID NO: 280.

11. The nucleic acid molecule of any of items 1-10, wherein the nucleic acid molecule comprises a nucleotide sequence as set forth in SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183, 137 185, 187, 189, 191, 193, 195, 197, 199, 201, 203, 205, 207, 209, 211, 213, 215, 217, 219, 221, 223, 225, 227, 229, 231, 233, 235, 237, 239, 241, 283, and 285.

12. A vector or expression cassette comprising the nucleic acid molecule of any of items 1-11.

13. A cell comprising the vector or expression cassette of item 12.

14. The cell of item 11, wherein the cell is a prokaryotic cell, preferably comprising Escherichia coli or a eukaryotic cell, preferably comprising a mammalian cell such as Chinese hamster ovary (CHO) cells, HEK293T Cells, and Expi293T cells, or an immune cell, preferably comprising a B cell, a T cell, a CAR T cell, a natural killer (NK) cell, or a neutrophil.

15. An amino acid sequence comprising at least one mutation at one or more amino acid positions of a human adenosine deaminase 1 (HsADA1) enzyme, wherein the mutated HsADA1 has increased or prolonged stability or catalytic activity at physiological conditions relative to a wildtype HsADA1, and wherein the HsADA1 has been mutated at one or more amino acid positions comprising a charged amino acid to a neutral or differently charged amino acid.

16. The amino acid molecule of item 15, wherein the amino acid molecule comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

17. The amino acid molecule of item 15 or 16, wherein the at least one mutation is in positions D8, S21, R33, N41, G45, V49, T57, G134, A148, N160, P163, K164, Q173, P189, L194, Q199, Q202, 1261, 1281, L283, and/or R341 of SEQ ID NO: 280.

18. The amino acid molecule of any of items 15-17, wherein the at least one mutation alters the charge of one or more of D8, S21, R33, N41, G45, V49, T57, G134, A148, N160, P163, K164, Q173, P189, L194, Q199, Q202, 1261, 1281, L283, and/or R341 of SEQ ID NO: 280.

19. The amino acid molecule of any of items 15-18, wherein the at least one mutation is selected from the group consisting of: N at position D8; A at position S21; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.

20. The amino acid molecule of any of items 15-19, wherein the at least one mutation comprises R33K, N41D, K164E, Q173N, L194F, Q199K, and/or Q202E.

21. The amino acid molecule of any of items 15-20, wherein the at least one mutation comprises one of (i) K164E, L194F, and Q199K; (ii) K164E, L194F, and Q173N; or (iii) K164E, L194F, and Q202E.

22. The amino acid molecule of any of items 15-21, further comprising at least one additional mutation in one or more of positions Y30, R32, D60, A71, E77, 179, E113, 1115, Al20, L124, E128, A131, R142, D143, V146, V166, Q175, 1180, E203, S207, 1209, A221, E222, K225, 12301, L236, L243, Q246, A247, R253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, 1115M, Al20T, L124V, E128D, A131D or S, R142Q, D143A, V1461, V166L, Q175K, 1180M, E203G, S207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

23. The amino acid molecule of any of items 15-22, further comprising at least one additional mutation in one or more of positions 160-202 of SEQ ID NO: 280.

24. The amino acid molecule of any of items 15-23, wherein the amino acid molecule comprises an amino acid sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

25. The amino acid molecule of any of items 15-24, wherein the amino acid molecule is fused to an IgG signal peptide at an N-terminal end of the amino acid molecule and to an IgG Fc region at a C-terminal end of the amino acid molecule.

26. A cell comprising the amino acid molecule of any of items 15-25.

27. The cell of item 26, wherein the cell is a prokaryotic cell, preferably comprising Escherichia coli or a eukaryotic cell, preferably comprising a mammalian cell such as Chinese hamster ovary (CHO) cells, HEK293T Cells, and Expi293T cells, or an immune cell, preferably comprising a B cell, a T cell, a CAR T cell, a natural killer (NK) cell, or a neutrophil.

28. A composition comprising an amino acid sequence comprising at least one mutation at one or more amino acid positions of a human adenosine deaminase 1 (HsADA1) enzyme, wherein the mutated HsADA1 has increased or prolonged stability or catalytic activity at physiological conditions relative to a wildtype HsADA1, and wherein the HsADA1 has been mutated at one or more amino acid positions comprising a charged amino acid to a neutral or differently charged amino acid.

29. The composition of item 28, wherein the amino acid molecule comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

30. The composition of items 28 or 29, wherein the at least one mutation is in positions D8, S21, R33, N41, G45, V49, T57, G134, A148, N160, P163, K164, Q173, P189, L194, Q199, Q202, 1261, 1281, L283, and/or R341 of SEQ ID NO: 280.

31. The composition of any of items 28-30, wherein the at least one mutation alters the charge of one or more of D8, S21, R33, N41, G45, V49, T57, G134, A148, N160, P163, K164, Q173, P189, L194, Q199, Q202, 1261, 1281, L283, and/or R341 of SEQ ID NO: 280.

32. The composition of any of items 28-31, wherein the at least one mutation is selected from the group consisting of: N at position D8; A at position S21; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.

33. The composition of any of items 28-32, wherein the at least one mutation comprises R33K, N41D, K164E, Q173N, L194F, Q199K, and/or Q202E.

34. The composition of any of items 28-33, wherein the at least one mutation comprises one of (i) K164E, L194F, and Q199K; (ii) K164E, L194F, and Q173N; or (iii) K164E, L194F, and Q202E.

35. The composition of any of items 28-34, further comprising at least one additional mutation in one or more of positions Y30, R32, D60, A71, E77, 179, E113, 1115, Al20, L124, E128, A131, R142, D143, V146, V166, Q175, 1180, E203, S207, 1209, A221, E222, K225, 12301, L236, L243, Q246, A247, R253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, 1115M, Al20T, L124V, E128D, A131D or S, R142Q, D143A, V1461, V166L, Q175K, 1180M, E203G, S207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

36. The composition of any of items 28-35 further comprising at least one additional mutation in one or more of positions 160-202 of SEQ ID NO: 280.

37. The composition of any of items 28-36, wherein the amino acid molecule comprises an amino acid sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

38. The composition of any of items 28-37, wherein the amino acid molecule is fused to an IgG signal peptide at an N-terminal end of the amino acid molecule and to an IgG Fc region at a C-terminal end of the amino acid molecule.

39. The composition of any of items 28-38, wherein a therapeutically effective amount of the composition is administered to a subject in need thereof to treat a cancer or tumor in the subject, optionally in combination with one or more cancer immunotherapies or adenosine depleting therapies, or wherein a therapeutically effective amount of the composition is administered to a subject in need thereof to treat ADA-SCID in the subject, optionally in combination with one or more ADA1 enzyme replacement therapies, optionally wherein the one or more cancer immunotherapies comprise PD-1 blockade via anti-PD-1 or anti-PD-L1 checkpoint inhibitor antibodies; an anti-CTLA-4 checkpoint inhibitor antibody; an anti-LAG3 antibody; an anti-TIM3 antibody; an anti-ICOS antibody; an anti-TIGIT antibody; an anti-GITR antibody; an anti-4-1BB antibody; an anti-0X40 antibody; an anti-CD40 antibody; an anti-CD38 antibody; an anti-B7-H3 antibody; or an anti-CD47 antibody.

40. A pharmaceutical composition comprising a therapeutically effective amount of an amino acid sequence comprising at least one mutation at one or more amino acid positions of an adenosine deaminase 1 (ADA1) enzyme, wherein the mutated ADA1 has increased or prolonged stability or catalytic activity at physiological conditions relative to a wildtype ADA1, and wherein the ADA1 has been mutated at one or more amino acid positions comprising a charged amino acid to a neutral or differently charged amino acid.

41. The pharmaceutical composition of item 40, wherein the amino acid molecule comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

42. The pharmaceutical composition of items 40 or 41, wherein the at least one mutation is in positions D8, S21, R33, N41, G45, V49, T57, G134, A148, N160, P163, K164, Q173, P189, L194, Q199, Q202, 1261, 1281, L283, and/or R341 of SEQ ID NO: 280.

43. The pharmaceutical composition of any of items 40-42, wherein the at least one mutation alters the charge of one or more of D8, S21, R33, N41, G45, V49, T57, G134, A148, N160, P163, K164, Q173, P189, L194, Q199, Q202, 1261, 1281, L283, and/or R341 of SEQ ID NO: 280.

44. The pharmaceutical composition of any of items 40-43, wherein the at least one mutation is selected from the group consisting of: N at position D8; A at position 521; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.

45. The pharmaceutical composition of any of items 40-44, wherein the at least one mutation comprises R33K, N41D, K164E, Q173N, L194F, Q199K, and/or Q202E.

46. The pharmaceutical composition of any of items 40-45, wherein the at least one mutation comprises one of (i) K164E, L194F, and Q199K; (ii) K164E, L194F, and Q173N; or (iii) K164E, L194F, and Q202E.

47. The pharmaceutical composition of any of items 40-46, further comprising at least one additional mutation in one or more of positions Y30, R32, D60, A71, E77, 179, E113, 1115, Al20, L124, E128, A131, R142, D143, V146, V166, Q175, 1180, E203, S207, 1209, A221, E222, K225, 12301, L236, L243, Q246, A247, 8253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, 1115M, Al20T, L124V, E128D, A131D or S, R142Q, D143A, V1461, V166L, Q175K, 1180M, E203G, S207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

48. The pharmaceutical composition of any of items 40-46, further comprising at least one additional mutation in one or more of positions 160-202 of SEQ ID NO: 280.

49. The pharmaceutical composition of any of items 40-48, wherein the amino acid molecule comprises an amino acid sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

50. The pharmaceutical composition of any of items 40-79, wherein a therapeutically effective amount of the composition is administered to a subject in need thereof to treat a cancer or tumor in the subject, optionally in combination with one or more cancer immunotherapies or adenosine depletion therapies, or wherein a therapeutically effective amount of the composition is administered to a subject in need thereof to treat ADA-SCID in the subject, optionally in combination with one or more ADA1 enzyme replacement therapies, optionally wherein the one or more cancer immunotherapies comprise PD-1 blockade via anti-PD-1 or anti-PD-L1 checkpoint inhibitor antibodies; an anti-CTLA-4 checkpoint inhibitor antibody; an anti-LAG3 antibody; an anti-TIM3 antibody; an anti-ICOS antibody; an anti-TIGIT antibody; an anti-GITR antibody; an anti-4-1BB antibody; an anti-OX40 antibody; an anti-CD40 antibody; an anti-CD38 antibody; an anti-B7-H3 antibody; or an anti-CD47 antibody.

51. The pharmaceutical composition of any of items 40-50, wherein the amino acid molecule is fused to an IgG signal peptide at an N-terminal end of the amino acid molecule and to an IgG Fc region at a C-terminal end of the amino acid molecule.

52. The pharmaceutical composition of any of items 40-51 further comprising an excipient and/or carrier.

53. The pharmaceutical composition of any of items 40-52, wherein the pharmaceutical composition is formulated for intratumoral, peritumoral, intradermal, subcutaneous, intravenous, or intraperitoneal administration.

54. A method of producing cells configured to express a human adenosine deaminase 1 (HsADA1) polypeptide, the method comprising:
introducing a nucleic acid molecule encoding the HsADA1 polypeptide on a vector and operably connected to an inducible promoter into the cells; and
culturing the cells under conditions suitable for expression of the HsADA1 polypeptide,
wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

55. The method of item 54, wherein the polypeptide comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

56. The method of item 54 or 55, wherein the at least one mutation is in positions D8, S21, R33, N41, G45, V49, T57, G134, A148, N160, P163, K164, Q173, P189, L194, Q199, Q202, 1261, 1281, L283, and/or R341 of SEQ ID NO: 280.

57. The method of any of items 54-56, wherein the at least one mutation alters the charge of one or more of D8, S21, R33, N41, G45, V49, T57, G134, A148, N160, P163, K164, Q173, P189, L194, Q199, Q202, 1261, 1281, L283, and/or R341 of SEQ ID NO: 280.

58. The method of any of items 54-57, wherein the at least one mutation is selected from the group consisting of: N at position D8; A at position S21; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.

59. The method of any of items 54-58, wherein the at least one mutation comprises R33K, N41D, K164E, Q173N, L194F, Q199K, and/or Q202E.

60. The method of any of items 54-59, wherein the at least one mutation comprises one of (i) K164E, L194F, and Q199K; (ii) K164E, L194F, and Q173N; or (iii) K164E, L194F, and Q202E.

61. The method of any of items 54-60, wherein the polypeptide further comprises at least one additional mutation in one or more of positions Y30, R32, D60, A71, E77, 179, E113, 1115, Al20, L124, E128, A131, R142, D143, V146, V166, Q175, 1180, E203, S207, 1209, A221, E222, K225, 12301, L236, L243, Q246, A247, 8253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, I115M, Al20T, L124V, E128D, A131D or S, R142Q, D143A, V1461, V166L, Q175K, 1180M, E203G, S207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

62. The method of any of items 54-61, further comprising at least one additional mutation in one or more of positions 160-202 of SEQ ID NO: 280.

63. The method of any of items 54-62, wherein the HsADA1 polypeptide comprises an amino acid sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

64. The method of any of items 54-63, wherein the cells are prokaryotic cells, preferably comprising Escherichia coli or eukaryotic cells, preferably comprising mammalian cells such as Chinese hamster ovary (CHO) cells, HEK293T Cells, and Expi293T cells, or an immune cell, preferably comprising a B cell, a T cell, a CAR T cell, a natural killer (NK) cell, or a neutrophil.

65. The method of any of items 54-64, wherein the HsADA1 polypeptide is fused to an IgG signal peptide at an N-terminal end of the HsADA1 polypeptide and to an IgG Fc region at a C-terminal end of the HsADA1 polypeptide.

67. A method of treating a cancer or tumor in a subject in need thereof; the method comprising:
administering to the subject a therapeutically effective amount of a composition comprising a human adenosine deaminase 1 (HsADA1) polypeptide,
wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

68. The method of item 67, wherein the HsADA1 polypeptide comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

69. The method of items 67 or 68, wherein the at least one mutation is in positions D8, S21, R33, N41, G45, V49, T57, G134, A148, N160, P163, K164, Q173, P189, L194, Q199, Q202, 1261, 1281, L283, and/or R341 of SEQ ID NO: 280.

70. The method of any of items 67-69, wherein the at least one mutation alters the charge of one or more of D8, S21, R33, N41, G45, V49, T57, G134, A148, N160, P163, K164, Q173, P189, L194, Q199, Q202, 1261, 1281, L283, and/or R341 of SEQ ID NO: 280.

71. The method of any of items 67-70, wherein the at least one mutation is selected from the group consisting of: N at position D8; A at position S21; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.

72. The method of any of items 67-71, wherein the at least one mutation comprises R33K, N41D, K164E, Q173N, L194F, Q199K, and/or Q202E.

73. The method of any of items 67-72, wherein the at least one mutation comprises one of (i) K164E, L194F, and Q199K; (ii) K164E, L194F, and Q173N; or (iii) K164E, L194F, and Q202E.

74. The method of any of items 67-73, wherein the HsADA1 polypeptide further comprises at least one additional mutation in one or more of positions Y30, R32, D60, A71, E77, 179, E113, 1115, Al20, L124, E128, A131, R142, D143, V146, V166, Q175, 1180, E203, S207, 1209, A221, E222, K225, 12301, L236, L243, Q246, A247, 8253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, I115M, Al20T, L124V, E128D, A131D or S, R142Q, D143A, V1461, V166L, Q175K, 1180M, E203G, S207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

75. The method of any of items 67-74, wherein the HsADA1 polypeptide further comprises at least one additional mutation in one or more of positions 160-202 of SEQ ID NO: 280.

76. The method of any of items 67-75, wherein the HsADA1 polypeptide comprises an amino acid sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

77. The method of any of items 67-76, wherein the HsADA1 polypeptide is fused to an IgG signal peptide at an N-terminal end of the HsADA1 polypeptide and to an IgG Fc region at a C-terminal end of the HsADA1 polypeptide.

78. The method of any of items 67-77, wherein the composition further comprises an excipient and/or a carrier.

79. The method of any of items 67-78, wherein the composition is formulated for intratumoral, peritumoral, intradermal, subcutaneous, intravenous, or intraperitoneal administration.

80. The method of any of items 67-79, wherein the composition is administered in combination with one or more cancer immunotherapies or with one or more adenosine depleting therapies, optionally wherein the one or more cancer immunotherapies comprise PD-1 blockade via anti-PD-1 or anti-PD-L1 checkpoint inhibitor antibodies; an anti-CTLA-4 checkpoint inhibitor antibody; an anti-LAG3 antibody; an anti-TIM3 antibody; an anti-ICOS antibody; an anti-TIGIT antibody; an anti-GITR antibody; an anti-4-1BB antibody; an anti-0X40 antibody; an anti-CD40 antibody; an anti-CD38 antibody; an anti-B7-H3 antibody; or an anti-CD47 antibody.

81. The method of any of items 67-80, wherein the cancer or tumor comprises non-small cell lung cancer (NSCLC) including NSCLC with high PD-1 expression (NSCLC-PD-1+), triple negative breast cancer (TNBC), and colon cancer, including unresectable colon cancer with mismatch repair deficiency (CRC-MMR−).

82. A method of treating adenosine deaminase-deficient severe combined immunodeficiency (ADA-SCID) in a subject in need thereof, the method comprising:
administering to the subject a therapeutically effective amount of a composition comprising a human adenosine deaminase 1 (HsADA1) polypeptide,
wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

83. The method of item 82, wherein the HsADA1 polypeptide comprises at least one mutation in positions D8, S21, R33, N41, G45, V49, T57, G134, A148, N160, P163, K164, Q173, Q174, P189, L194, Q199, Q202, 1261, 1281, L283, and/or R341 of SEQ ID NO: 280.

84. The method of item 82 or 83, wherein the at least one mutation is in positions D8, 521, R33, N41, G45, V49, T57, G134, A148, N160, P163, K164, Q173, P189, L194, Q199, Q202, 261, 281, L283, and/or R341 of SEQ ID NO: 280.

85. The method of any of items 82-84, wherein the at least one mutation alters the charge of one or more of D8, S21, R33, N41, G45, V49, T57, G134, A148, N160, P163, K164, Q173, P189, L194, Q199, Q202, 1261, 1281, L283, and/or R341 of SEQ ID NO: 280.

86. The method of any of items 82-85, wherein the at least one mutation is selected from the group consisting of: N at position D8; A at position S21; K at position R33; D at position N41; E at position G45; I at position V49; S at position T57; N at position G134; V at position A148; S at position N160; S at position P163; E at position K164; N or E at position Q173; E at position Q174; E at position P189; F at position L194; K at position Q199; E at position Q202; V at position 1261; V at position 1281; F at position L283; and K at position R341.85. The method of claim 81, wherein the at least one mutation comprises R33K, N41D, K164E, Q173N, L194F, Q199K, and/or Q202E.

87. The method of any of items 82-86, wherein the at least one mutation comprises one of (i) K164E, L194F, and Q199K; (ii) K164E, L194F, and Q173N; or (iii) K164E, L194F, and Q202E.

88. The method of any of items 82-87, wherein the HsADA1 polypeptide further comprises at least one additional mutation in one or more of positions Y30, R32, D60, A71, E77, 179, E113, Ills, Al20, L124, E128, A131, R142, D143, V146, V166, Q175, 1180, E203, S207, 1209, A221, E222, K225, 12301, L236, L243, Q246, A247, 8253, Q254, K273, D275, E277, A279, Q287, A288, R313, D314, D338, D345, L346, K349, A350, and/or G352, optionally wherein the at least one additional mutation is selected from the group consisting of Y30F, R32K, D60E or G, A71V, E77D, I79V, El 13D, I115M, Al20T, L124V, E128D, A131D or S, R142Q, D143A, V1461, V166L, Q175K, 1180M, E203G, 5207N, 1209V, A221P, E222N, K225R, 1230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

89. The method of any of items 82-88, wherein the HsADA1 polypeptide further comprises at least one additional mutation in one or more of positions 160-202 of SEQ ID NO: 280.

90. The method of any of items 82-89, wherein the HsADA1 polypeptide comprises an amino acid sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 149 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 12, 214, 216, 218, 220, 222, 224, 226, 228, 230, 232, 234, 236, 238, 240, 242, 284, and 286.

91. The method of any of items 82-90, wherein the HsADA1 polypeptide is fused to an IgG signal peptide at an N-terminal end of the HsADA1 polypeptide and to an IgG Fc region at a C-terminal end of the HsADA1 polypeptide.

92. The method of any of items 82-91, wherein the composition further comprises an excipient and/or a carrier.

93. The method of any of items 82-92, wherein the composition is formulated for subcutaneous, intradermal, intravenous, or intraperitoneal administration.

94. The method of any of items 82-93, wherein the composition is administered in combination with one or more ADA1 enzyme replacement therapeutics.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

1. A nucleic acid molecule encoding human adenosine deaminase 1 (HsADA1), wherein the HsADA1 has been mutated to have increased or prolonged stability or catalytic activity at physiological conditions relative to a wildtype HsADA1.

2. The nucleic acid molecule of claim 1, wherein the HsADA1 has been mutated at one or more amino acid positions comprising a charged amino acid to a neutral or differently charged amino acid.

3. The nucleic acid molecule of claim 1, wherein the nucleic acid molecule comprises at least one mutation in position D8, S21, R33, N41, G45, V49, T57, G134, A148, N160, P163, K164, Q173, Q174, P189, L194, Q199, Q202, I261, I281, L283, and/or R341 of SEQ ID NO: 280.

4. The nucleic acid molecule of claim 1, wherein at least one mutation is in position D8, S21, R33, N41, G45, V49, T57, G134, A148, N160, P163, K164, Q173, P189, L194, Q199, Q202, I261, I281, L283, and/or R341 of SEQ ID NO: 280.

5. The nucleic acid molecule of claim 1, wherein at least one mutation alters the charge of one or more of positions D8, S21, R33, N41, G45, V49, T57, G134, A148, N160, P163, K164, Q173, P189, L194, Q199, Q202, I261, I281, L283, and/or R341 of SEQ ID NO: 280.

6. The nucleic acid molecule of claim 3, wherein at least one mutation is selected from the group consisting of:
N at position D8;
A at position S21;
K at position R33;
D at position N41;
E at position G45;
I at position V49;
S at position T57;
N at position G134;
V at position A148;
S at position N160;
S at position P163;
E at position K164;
N or E at position Q173;
E at position Q174;
E at position P189;
F at position L194;
K at position Q199;
E at position Q202;
V at position I261;
V at position I281;
F at position L283; and
K at position R341.

7. The nucleic acid molecule of claim 6, wherein at least one mutation comprises R33K, N41D, K164E, Q173N, L194F, Q199K, and/or Q202E.

8. The nucleic acid molecule of claim 6, wherein at least one mutation comprises one of (i) K164E, L194F, and Q199K; (ii) K164E, L194F, and Q173N; or (iii) K164E, L194F, and Q202E.

9. The nucleic acid molecule of claim 3 further comprising at least one additional mutation in one or more of positions Y30, R32, D60, A71, E77, I79, E113, I115, A120, L124, E128, A131, R142, D143, V146, V166, Q175, I180, E203, S207, I209, A221, E222, K225, I2301, L236, L243, Q246, A247, R253, Q254, K273, D275, E277, A279, Q287, A288, R313, D314, D338, D345, L346, K349, A350, and/or G352;
optionally wherein at least one of the additional mutations is selected from the group consisting of Y30F, R32K, D60E or G, A71V, E77D, I79V, E113D, I115M, A120T, L124V, E128D, A131D or S, R142Q, D143A, V146I, V166L, Q175K, I180M, E203G, S207N, I209V, A221P, E222N, K225R, I230T, L236V, L243I, Q246E or T, A247T, R253L, Q254K, K273D, D275K, E277T, A279P, Q287K, A288V, R313K or N, D314E, D338E, D345E, L346R, K349R, A350E, and G352Q.

10. The nucleic acid molecule of claim 3 further comprising at least one additional mutation in one or more of positions 160-202 of SEQ ID NO: 280.

11. The nucleic acid molecule of claim 1, wherein the nucleic acid molecule comprises a nucleotide sequence as set forth in SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 283, and 285.

12. A vector or expression cassette comprising the nucleic acid molecule of claim 1.

13. A cell comprising the vector or expression cassette of claim 12.

14. The cell of claim 13, wherein the cell is a prokaryotic cell, preferably comprising Escherichia coli or a eukaryotic cell, preferably comprising a mammalian cell such as Chinese hamster ovary (CHO) cells, HEK293T Cells, and Expi293T cells, or an immune cell, preferably comprising a B cell, a T cell, a CAR T cell, a natural killer (NK) cell, or a neutrophil.

15. An amino acid sequence comprising at least one mutation at one or more amino acid positions of a human adenosine deaminase 1 (HsADA1) enzyme, wherein the mutated HsADA1 has increased or prolonged stability or catalytic activity at physiological conditions relative to a wildtype HsADA1, and wherein the HsADA1 has been mutated at one or more amino acid positions comprising a charged amino acid to a neutral or differently charged amino acid.

16.-25. (canceled)

26. A cell comprising the amino acid sequence of claim 15.

27. (canceled)

28. A composition comprising the amino acid sequence of claim 15.

29.-37. (canceled)

38. The composition of claim 28, wherein the amino acid sequence is fused to an IgG signal peptide at an N-terminal end of the amino acid sequence and to an IgG Fc region at a C-terminal end of the amino acid sequence.

39. The composition of claim 28, wherein a therapeutically effective amount of the composition is administered to a subject in need thereof to treat a cancer or tumor in the subject, optionally in combination with one or more cancer immunotherapies or adenosine depletion therapies, or wherein a therapeutically effective amount of the composition is administered to a subject in need thereof to treat ADA-SCID in the subject, optionally in combination with one or more ADA1 enzyme replacement therapies, optionally wherein the one or more cancer immunotherapies comprise PD-1 blockade via anti-PD-1 or anti-PD-L1 checkpoint inhibitor antibodies; an anti-CTLA-4 checkpoint inhibitor antibody; an anti-LAG3 antibody; an anti-TIM3 antibody; an anti-ICOS antibody; an anti-TIGIT antibody; an anti-GITR antibody; an anti-4-1BB antibody; an anti-OX40 antibody; an anti-CD40 antibody; an anti-CD38 antibody; an anti-B7-H3 antibody; or an anti-CD47 antibody.

40. A pharmaceutical composition comprising a therapeutically effective amount of the amino acid sequence of claim 15.

41.-53. (canceled)

54. A method of producing cells configured to express a human adenosine deaminase 1 (HsADA1) polypeptide, the method comprising:
introducing a nucleic acid molecule encoding the HsADA1 polypeptide on a vector and operably connected to an inducible promoter into the cells; and
culturing the cells under conditions suitable for expression of the HsADA1 polypeptide;
wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

55.-65. (canceled)

66. A method comprising:
administering to the subject a therapeutically effective amount of a composition comprising a human adenosine deaminase 1 (HsADA1) polypeptide;
wherein the HsADA1 polypeptide comprises at least one mutation at one or more amino acid positions comprising a charged amino acid to a neutral or differently charged amino acid, and wherein the mutated HsADA1 polypeptide has increased or prolonged stability or catalytic activity at physiological conditions relative to a wildtype HsADA1 polypeptide.

67.-80. (canceled)

81. The method of claim 66, wherein the method is selected from a group consisting of:
treating a cancer or tumor in a subject in need thereof; and
treating adenosine deaminase-deficient severe combined immunodeficiency (ADA-SCID) in a subject in need thereof.

82.-94. (canceled)
